(19) United States
(12) Patent Application Publication
(10) Pub. No.: US 2014/0079713 A1
(43) Pub. Date: Mar. 20, 2014

(54) MODULATION OF THE SONIC HEDGEHOG PATHWAY AND USES THEREOF

(71) Applicants: Frederic Charron, Westmount (CA); Luisa Izzi, Montreal (CA); Steves Morin, Montreal (CA)

(72) Inventors: Frederic Charron, Westmount (CA); Luisa Izzi, Montreal (CA); Steves Morin, Montreal (CA)

(73) Assignee: INSTITUT DE RECHERCHES CLINIQUES DE MONTREAL, MONTREAL, QC (CA)

(21) Appl. No.: 14/115,177

(22) PCT Filed: May 2, 2012
(86) PCT No.: PCT/CA2012/050284

§ 371 Date: Nov. 1, 2013

Related U.S. Application Data
(60) Provisional application No. 61/481,502, filed on May 2, 2011.

(51) Int. Cl. A61K 39/395; G01N 33/50; A61K 38/17

(52) U.S. Cl. CPC A61K 39/39558; A61K 38/17; G01N 33/5011
USPC 424/139.1; 514/19.3; 514/19.4; 514/19.5; 435/375; 435/32

(57) ABSTRACT

A method for modulating Sonic hedgehog (Shh)-mediated signalling and proliferation based on agents that modulates the binding of GAS1, BOC and/or CDON to Ptch1 is disclosed. Methods and compositions for treating cancer using agents that inhibit the binding of GAS1, BOC and/or CDON to Ptch1 are also disclosed. Methods for identifying agents that may be used to inhibit Shh-mediated signalling/proliferation and cancer based on their capacity to inhibit the binding of GAS1, BOC and/or CDON to Ptch1 are also disclosed.

### CROSS-REFERENCE TO RELATED APPLICATION

The present application claims the benefit of U.S. provisional patent application Ser. No. 61/481,502, filed on May 2, 2011, the content of which is incorporated herein by reference in its entirety.

### TECHNICAL FIELD

The present invention generally relates to Sonic Hedgehog signalling, and more particularly to methods for modulating Sonic Hedgehog signalling and its associated effects.

### BACKGROUND ART

Hh proteins are key molecules for diverse tissue patterning processes in both invertebrates and vertebrates. For example, in Drosophila, Hh is crucial for the development of a segmented body plan and the patterning of imaginal tissues, whereas in vertebrates Sonic hedgehog (Shh) functions to pattern limb buds and promote cell fate specification, proliferation, and axon guidance in the central nervous system (Charron and Tessier-Lavigne, 2005; Dessaud et al., 2008; Fuccillo et al., 2006; Ingham and Placzek, 2006; Jiang and Hui, 2008; Varjosalo and Taipale, 2008). Shh is synthesized as a 45 kDa pro-protein and post-translational modifications generate a biologically active 19 kDa N-terminal fragment (Lewis et al., 2001). Shh initiates signaling by binding the 12-pass transmembrane protein Patched1 (PTCH1). Upon Shh binding, the inhibition exerted by PTCH1 on the 7-pass transmembrane protein Smoothened (Smo) is relieved, eliciting a signaling cascade which ultimately leads to Gli-mediated transcription (Wilson and Chuang, 2010).

However, it is still unclear how the Shh signalling pathway and its associated biological effects are regulated.

Also, the Hedgehog pathway has been shown to be involved in tumor development and constitute an attractive strategy in anticancer therapy. There is thus a need for the development of agents and methods that interfere with this pathway for cancer treatment.

The present description refers to a number of documents, the content of which is herein incorporated by reference in their entirety.

### SUMMARY OF THE INVENTION

In an aspect, the present invention provides a method for inhibiting Sonic hedgehog (Shh)-mediated signalling and/or proliferation in a cell, said method comprising contacting said cell with an agent that inhibits the binding of a Growth Arrest Specific 1 (GAS1) polypeptide, a cell-adhesion-molecule-related/downregulated by oncogenes (CDON) polypeptide and/or biregional CDON-binding protein (BOC) polypeptide to a Patched1 (PTCH1) polypeptide, in said cell.

In another aspect, the present invention provides a method for preventing and/or treating cancer in a subject, said method comprising administering to said subject an effective amount of an agent that inhibits the binding of a Growth Arrest Specific 1 (GAS1) polypeptide, a cell-adhesion-molecule-related/downregulated by oncogenes (CDON) polypeptide and/or biregional CDON-binding protein (BOC) polypeptide to a Patched1 (PTCH1) polypeptide.

In another aspect, the present invention provides the use of an agent that inhibits the binding of a Growth Arrest Specific 1 (GAS1) polypeptide, a cell-adhesion-molecule-related/downregulated by oncogenes (CDON) polypeptide and/or biregional CDON-binding protein (BOC) polypeptide to a Patched1 (PTCH1) polypeptide for inhibiting Sonic hedgehog (Shh)-mediated signalling and/or proliferation in a cell.

In another aspect, the present invention provides the use of an agent that inhibits the binding of a Growth Arrest Specific 1 (GAS1) polypeptide, a cell-adhesion-molecule-related/downregulated by oncogenes (CDON) polypeptide and/or biregional CDON-binding protein (BOC) polypeptide to a Patched1 (PTCH1) polypeptide for the preparation of a medicament for inhibiting Sonic hedgehog (Shh)-mediated signalling and/or proliferation in a cell.

In another aspect, the present invention provides the use of an agent that inhibits the binding of a Growth Arrest Specific 1 (GAS1) polypeptide, a cell-adhesion-molecule-related/downregulated by oncogenes (CDON) polypeptide and/or biregional CDON-binding protein (BOC) polypeptide to a Patched1 (PTCH1) polypeptide for preventing and/or treating cancer in a subject.

In another aspect, the present invention provides the use of an agent that inhibits the binding of a Growth Arrest Specific 1 (GAS1) polypeptide, a cell-adhesion-molecule-related/downregulated by oncogenes (CDON) polypeptide and/or biregional CDON-binding protein (BOC) polypeptide to a Patched1 (PTCH1) polypeptide for the preparation of a medicament for preventing and/or treating cancer in a subject.

In another aspect, the present invention provides a method of identifying an agent that may be useful for inhibiting Sonic hedgehog (Shh)-mediated signalling and/or proliferation in a cell, said method comprising determining the binding of a Growth Arrest Specific 1 (GAS1), a cell-adhesion-molecule-related/downregulated by oncogenes (CDON) polypeptide and/or biregional CDON-binding protein (BOC) polypeptide to a Patched1 (PTCH1) polypeptide, in the presence of said agent, wherein a lower or decreased binding in the presence of said agent is indicative that said agent may be useful for inhibiting Sonic hedgehog (Shh)-mediated signalling and/or proliferation in a cell.

In another aspect, the present invention provides a method of identifying an agent that may be useful for preventing and/or treating cancer in a subject, said method comprising determining the binding of a Growth Arrest Specific 1 (GAS1), a cell-adhesion-molecule-related/downregulated by oncogenes (CDON) polypeptide and/or biregional CDON-binding protein (BOC) polypeptide to a Patched1 (PTCH1) polypeptide, in the presence of said agent, wherein a lower or decreased binding in the presence of said agent is indicative that said agent may be useful for preventing and/or treating cancer in a subject.

In an embodiment, the above-mentioned agent inhibits the binding of a GAS1 polypeptide to a PTCH1 polypeptide.

In a further embodiment, the above-mentioned agent is a polypeptide comprising a sequence derived from the sequence of a GAS1 polypeptide. In a further embodiment, the above-mentioned polypeptide comprises a sequence derived from a sequence located in a region defined by residues 45 to 317 of SEQ ID NO:6.

In another embodiment, the above-mentioned agent is an antibody directed against GAS1. In a further embodiment, the above-mentioned antibody directed against GAS1 binds to an epitope located within a region defined by residues 45 to 317 of SEQ ID NO:6.

In another embodiment, the above-mentioned agent inhibits the binding of a BOC polypeptide to a PTCH1 polypeptide.

In an embodiment, the above-mentioned is a polypeptide comprising a sequence derived from the sequence of a BOC polypeptide. In a further embodiment, the above-mentioned agent is a polypeptide comprising a sequence derived from a sequence located in a region defined by residues 460 to 700 of SEQ ID NO:2. In a further embodiment, the above-mentioned agent is a polypeptide comprising a sequence derived from a sequence located in a region defined by residues 460 to 570 and/or 600 to 700 of SEQ ID NO:2.

In another embodiment, the above-mentioned agent is an antibody directed against BOC. In a further embodiment, the above-mentioned antibody directed against BOC binds to an epitope located within a region defined by residues 460 to 700 of SEQ ID NO:2. In a further embodiment, the above-mentioned antibody directed against BOC binds to an epitope within a region defined by residues 460 to 570 and/or 600 to 700 of SEQ ID NO:2.

In an embodiment, the above-mentioned agent inhibits the binding of a CDON polypeptide to a PTCH1 polypeptide.

In an embodiment, the above-mentioned agent is a polypeptide comprising a sequence derived from the sequence of a CDON polypeptide. In a further embodiment, the above-mentioned agent is a polypeptide comprising a sequence derived from a sequence located in a region defined by residues 575 to 815 of SEQ ID NO:4. In a further embodiment, the above-mentioned agent is a polypeptide comprising a sequence derived from a sequence located in a region defined by residues 575 to 675 and/or 720 to 815 of SEQ ID NO:4.

In another embodiment, the above-mentioned agent is an antibody directed against CDON. In a further embodiment, the above-mentioned antibody directed against CDON binds to an epitope located within a region defined by residues 575 to 815 of SEQ ID NO:4. In a further embodiment, the above-mentioned antibody directed against BOC binds to an epitope within a region defined by residues 575 to 675 and/or 720 to 815 of SEQ ID NO:4.

In another embodiment, the above-mentioned agent is a polypeptide comprising a sequence derived from the sequence of a PTCH1 polypeptide. In a further embodiment, the above-mentioned agent is a polypeptide comprising a sequence derived from a sequence located in a region defined by residues 122-436, 494-501, 569-577, 1077-1083 or 1142-1154 of SEQ ID NO:8.

In another embodiment, the above-mentioned agent is an antibody directed against PTCH1. In a further embodiment, the above-mentioned antibody directed against PTCH1 binds to an epitope located within a region defined by residues 122-436, 494-501, 569-577, 1077-1083 or 1142-1154 of SEQ ID NO:8.

In an embodiment, the above-mentioned cell is a tumor cell.

In another embodiment, the above-mentioned cell is a neural cell, in a further embodiment a neuron progenitor, and in yet a further embodiment a cerebellar granular neuron progenitor (CGNP).

In an embodiment, the above-mentioned cancer is associated with hedgehog signalling (e.g., Sonic hedgehog-mediated signaling).

In an embodiment, the above-mentioned cancer is associated with GAS1, CDON, BOC and/or PTCH1 expression and/or activity.

In an embodiment, the above-mentioned cancer is a brain tumor, an ovary tumor, a breast tumor, a glioblastoma, a skin tumor, a meningioma, an astrocytoma, a liver tumor, a prostate carcinoma, a bladder tumor, a lung tumor, a lymph node lymphoma, a vascular endothelium hemangioma, a kidney carcinoma or a thyroid follicular adenoma.

In an embodiment, the above-mentioned brain tumor is a neuroectodermal tumor, in a further embodiment a medulloblastoma.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIGS. 1A to 1C show Boc and Cdon expression in the developing cerebellum. FIG. 1A is a diagram of the developing cerebellum at E14.5 and E18.5. Immunostaining of Boc and Cdon in sagittal sections of the developing mouse cerebellum shows that Boc is expressed in the EGL, ChP and RL at E14.5 and expression is maintained in the EGL and ChP at E18.5. Cdon is expressed in the RL and ChP at E14.5 and E18.5. FIG. 1B shows that at P6, Xgal staining (left, counterstained with nuclear fast red) and immunofluorescence on sagittal cerebellum sections from Boc^{+/-} and Cdon^{+/-} βgeo (β-galactosidase-neomycin) mice reveal Boc and Cdon expression. Boc is expressed in Lim1⁺ cells (B,C) and Cdon in the ChP (B). FIG. 1C shows P3 WT mouse cerebellum sections co-immunolabeled with Boc and various cerebellar cell markers showing high Boc expression in proliferating CGNPs (Lim1⁺, Pax6⁺) and lower expression in PC (CaBP⁺, Lim1⁺) and differentiated granule cells (Pax6⁺ in the IGL). Scale bars: (A) 100 μm (B) 250 μm, (C) 50 μm. EGL, external germinal layer; IGL, internal granular layer; ChP, choroid plexus; RL, rhombic lip; PC, Purkinje cells; CGNP, cerebellar granular neurons; CaBP, Calbindin; VZ, ventricular zone;

FIGS. 2A to 2I show Boc^{-/-} mice have a smaller cerebellum than control mice. FIG. 2A: Comparison of whole cerebella and (C) sagittal sections from adult Boc^{+/-} and Boc^{-/-} mice. FIG. 2B: Cerebellum weight and normalized cerebellum weight relative to body weight. n=11 cerebella/group. FIG. 2D: Cerebellum surface area (left) and IGL surface area (right) measured from three medio-lateral matching levels. n=4 cerebella/group. FIG. 2E: Apoptotic CGNPs from P3 Boc^{+/-} and Boc^{-/-} mice visualized by TUNEL staining. FIG. 2F: Number of apoptotic cells/mm² in the EGL from TUNEL-stained sections from 5 Boc^{+/-} and 4 Boc^{-/-} mice. FIG. 2G: Proliferating CGNPs in the EGL from P3 Boc^{+/-} and Boc^{-/-} mice visualized by anti-BrdU staining. FIG. 2H: (left) Percentage of BrdU⁺ cells in the EGL. n=4 animals/group. (right) Number of pH3⁺ cells/mm² in the EGL of 5 Boc^{+/-} and 4 Boc^{-/-} P3 mice. FIG. 2I: CGNPs purified from Boc^{+/+}, Boc^{+/-} and Boc^{-/-} mice cerebella at P4 were cultured in the presence of 0-90 nM ShhN. Proliferating cells were measured by ³H-thymidine incorporation. Data is represented as fold induction in CGNP proliferation compared to untreated cells. Scale bars: (A) 2 mm, (C) 1 mm, (E,G) 250 μm. IGL, internal granular layer; EGL, external germinal layer. p values measured from Student's t-test (B,D,F,H) and two-way ANOVA (I);

FIGS. 3A to 3D show Gas1 expression in the developing cerebellum. FIG. 3A: Immunostaining of sagittal sections showing Gas1 expression in the RL and EGL of the developing cerebellum at E14.5 and E18.5. FIGS. 3B and C: P4-6 WT cerebellum sections immunostained for Gas1 and various cerebellar cell markers. Gas1 is highly expressed by proliferating CGNPs (Lim1⁺ cells; Pax6⁺ in EGL). FIG. 3D: Immunostaining of E18.5 cerebellum sections from Math1-Cre;mTmG mice show that Boc and Gas1 are expressed in Math1⁺,GFP⁺ CGNPs in the EGL. EGL, external germinal layer; IGL, internal granular layer; PC, Purkinje cells; RL, rhombic lip; VZ, ventricular zone; ChP, choroid plexus. Scale bars: (A, C and D) 100 μm, (3B) 500 μm;

FIGS. 4A to F show Shh-dependent proliferation is completely lost in Gas1^{-/-};Boc^{-/-} CGNPs. FIG. 4A: Haematoxylin-eosin staining on sagittal sections of E18.5 cerebellum revealing a thinner EGL in Gas1^{-/-};Boc^{-/-} cerebella than control. Anti-pH3 immunostaining of sagittal sections of Gas1^{+/-};Boc^{-/-} and Gas1^{-/-};Boc^{-/-} cerebella counterstained with DAPI. RNA in situ hybridization showing the loss of expression of the Shh transcriptional target Gil1 in Gas1^{-/-};Boc^{-/-} cerebella at E18.5. FIG. 4B: Quantification of: cerebellum surface area, EGL surface area, pH3⁺ cells in EGL, pH3⁺ cells per pm^{t}, and EGL thickness along the postero-anterior axis, n=4 animals/group. FIG. 4C: CGNPs purified from Gas1^{+/+};Boc^{+/+} (n=3), Gas1^{+/-};Boc^{+/+} (n=4) and Gas1^{-/-};Boc^{+/+} (n=4) mice cerebella at E18.5 were cultured with 0, 3, 10, 30 nM ShhN. Proliferating cells were visualized by immunostaining with an anti-Ki67 antibody. Data is represented as fold CGNP proliferation over untreated control (C, D and E) or DMSO control (F). FIG. 4D: Similar to FIG. 3C but CGNPs were purified from Gas1^{+/+};Boc^{-/-} (n=3), Gas1^{+/-};Boc^{-/-} (n=3) and Gas1^{-/-};Boc^{-/-} (n=3) mice cerebella at E18.5. FIG. 4E: CGNPs were purified from control (Ctl; Gas1^{+/+};Boc^{-/-} and Gas1^{+/-};Boc^{-/-}) (n=3) and Gas1^{-/-};Boc^{-/-} (n=3) mice cerebella at E18.5 and treated with 0, 20, 50 or 100 ng/ml of IGF-I. FIG. 4F: Similar to FIG. 4E but CGNPs were treated with either DMSO, 0.150 μM purmorphamine, or 30 nM ShhN. p values measured from Student's t-test (B), two-way ANOVA (C, E), and ANOVA (D, F). EGL, external germinal layer. Scale bars: top two rows=500 μm, bottom two rows=100 μm;

FIGS. 5A to 5G show that GAS1 and BOC interact with PTCH1. FIG. 5A: BOC and GAS1 interact with PTCH1. COS7 cells were transfected with the indicated constructs and lysates were immunoprecipitated (IP) with an anti-GFP antibody and immunoblotted (IB) with anti-GAS1, anti-Flag or anti-GFP antibodies. FIG. 5B: BOC interacts with PTCH1 in a constitutive manner. COS7 cells expressing PTCH1-HA and BOC-Flag were treated with ShhN and subjected to anti-HA IP and either anti-Flag or anti-HA IB. FIG. 5C: The PTCH1 L2 region is not required for the BOC-PTCH1 interaction. Anti-HA IP was performed on COS7 lysates expressing Boc-Flag and PTCH1-HA or PTCH1ΔL2-followed by anti-Flag or anti-HA IB. FIG. 5D: The BOC cytoplasmic tail is not involved in the BOC-PTCH1 interaction. COS7 cells expressing PTCH1-HA and BOC-GFP or BOCΔcyto-GFP were subjected to anti-GFP IP and either anti-HA or anti-GFP IB. FIGS. 5E and F: PTCH1 forms receptor complexes with either BOC or GAS1 but not both. BOC-Flag was co-transfected with GAS1 with or without PTCH1-GFP in COS7 cells. FIG. 5E: Lysates were IP with anti-Flag antibodies, followed by anti-GAS1 or anti-GFP IB. FIG. 5F: Lysates were first immunoprecipated (IP #1) with anti-GAS1 antibodies. Supernatants from IP#1 were subjected to a second immunoprecipitation (IP #2) with anti-Flag antibodies. Both anti-GAS1 (IP #1) and anti-Flag (IP #2) immunoprecipitates were IB with anti-GAS1, anti-Flag and anti-GFP antibodies. See FIG. 11 for a schematic of this experiment. In FIGs. A to F, protein expression inputs were verified by IB with the indicated antibodies. ns, non-specific. FIG. 5G (left): Diagram of WT BOC, BOC-Fc and BOC-Fc mutant proteins. FIG. 5G (right): The BOC-PTCH1 interaction is mediated by the BOC FNIII(ab) domains. COS7 cells expressing PTCH1-GFP were incubated with conditioned mediated containing BOC-Fc proteins. Bound proteins were labeled with HRP-conjugated anti-Fc antibody and peroxidase activity measured;

FIGS. 6A to 6F show a Shh mutant which binds PTCH1 but which fails to bind BOC, CDON and GAS1, and does not induce Shh signaling. FIGS. 6A to C: Structural representation of ShhN, with residues E90 (FIG. 6A) and R154 (FIG. 6B) indicated. Amino acids important for PTCH1 binding are colored in black while residues dispensable for the PTCH1-Shh interaction are illustrated in dark grey. FIG. 6D: Anti-ShhN immunoblots showing the expression of purified ShhN WT, E90A, and R154E in comparison to ShhN C24II (top panel). Anti-ShhN immunoblot showing conditioned medium (CM) containing ShhN-AP WT, E90A, and R154E in comparison to ShhN C24II (bottom panel). FIG. 6E: Stimulation of Gli-luciferase reporter activity in C3H 10T½ cells treated with 17, 50 and 150 nM ShhN WT, E90A or R154E mutants. FIG. 6F: Differentiation of C3H 10T½ cells treated with 12.5, 25 and 50 nM ShhN WT, E90A or R154E mutants. In FIGS. 6E and F, each condition was performed in duplicate, n=3. FIG. 6G: Induction in rat P4 CGNP proliferation upon treatment with 6.25, 12.5, 25 and 50 nM of ShhN WT, E90A or R154E mutants. Proliferation was measured by ³H-thymidine incorporation. Each condition was performed in triplicate, n=3. In FIGS. 6E to G, two-way ANOVA comparing the activity of ShhN E90A to ShhN WT was performed. FIG. 6H: Table summarizing binding of ShhN mutants to PTCH1, BOC, CDON and GAS1 and their functional activity;

FIG. 7 shows that Shh signaling involves the BOC, CDON and GAS1 receptors. (Left) In the absence of Shh, BOC, CDON and GAS1 interact with PTCH1 to form BOC/PTCH1, CDON/PTCH1 or GAS1/PTCH1 complexes. The BOC/PTCH1 interaction is mediated by the FNIIIa and FNIIIb domains of BOC. (Right) Shh binding to BOC/PTCH1, CDON/PTCH1, and GAS1/PTCH1 receptor complexes leads to the de-repression of Smo, which in turn activates signal transduction events that result in Gli-mediated transcription. In the case of BOC and CDON, the interaction with Shh is direct and is mediated by the FNIIIc domain of BOC and CDON. Unlike Ihog and Boi which promote the relocalization of Ptc to the surface of Drosophila cells, BOC and GAS1 do not seem to relocalize PTCH1 to the surface of mammalian cells (FIGS. 12A-C);

FIG. 8 shows that Cdon inactivation does not affect the proliferation of Boc−/− CGNPs. Sagittal sections of E18.5 mouse cerebellum were immunolabeled with an anti-phospho-histone H3 (pH3) antibody. Consistent with the lack of Cdon expression in CGNPs, quantitation of pH3+ cells in the EGL showed that loss of Cdon does not affect proliferation of Boc−/− CGNPs;

FIGS. 9A to 9E: CGNPs are specified in Gas1−/−;Boc−/− cerebellum and Cdon expression is not upregulated in Gas1−/−;Boc−/− cerebella. Sagittal sections of Gas1+/−;Boc−/− and Gas1−/−;Boc−/− E18.5 mouse cerebellum were immunostained for two CGNP markers, Lim1 (FIG. 9A) or Pax6 (FIG. 9B). Although reduced in numbers, CGNPs are specified in Gas1−/−;Boc−/− mice. FIG. 9C: Cdon immunostaining showing that Cdon expression is not changed in Gas1−/−;Boc−/− compared to Gas1+/−;Boc−/− cerebellum. FIG. 9D: Methodology used for the quantification of the EGL thickness along the postero-anterior axis of the cerebellum. The EGL thickness was measured every 10°, starting from the border of the rhombic lip and the EGL, towards the anterior pole of the cerebellum. The results are shown in FIG. 4B. FIG. 9E: CGNPs purified from control (Ctl; Gas1+/+;Boc−/− and Gas1+/−;Boc−/−) (n=5) and Gas1−/−;Boc−/− (n=5) mice cerebella at E18.5 were cultured with 0, 3, 10, 30, 60, 90 nM ShhN. Proliferating cells were visualized by immunostaining with an anti-Ki67 antibody. Data is represented as fold CGNP proliferation over untreated control. p values measured from ANOVA followed by Bonferroni post-test. EGL, external germinal layer;

FIGS. 10A to 10C: GAS1 and BOC interact with PTCH1 but not Disp1 or Smo. FIGS. 10A and B: COS7 cells were transfected with the indicated constructs and lysates were immunoprecipitated (IP) with an anti-GFP antibody and immunoblotted (IB) with anti-GAS1, anti-Flag or anti-GFP antibodies. FIG. 10C: Cell surface binding assays were performed by incubating conditioned media containing BOC-Fc proteins with COS7 cells transfected with either PTCH1-GFP, Disp1-YFP or Smo-GFP. Bound BOC-Fc proteins were labeled with HRP-conjugated anti-Fc antibody and peroxidase activity measured. Data is represented as mean±standard deviation. * The anti-GFP antibody used also recognizes YFP;

FIG. 11 is a schematic diagram illustrating the methodology used for the double immunoprecipitation assays shown in FIG. 5F. Whole cell lysates from COS7 cells transfected with PTCH1-GFP, BOC-Flag and GAS1 were first subjected to immunoprecipitation with anti-GAS1 antibody (IP#1). Following the anti-GAS1 immunoprecipitation, supernatants from the first immunoprecipitation were subjected to a precipitation with anti-Flag antibody (IP#2). Both anti-GAS1 (IP#1) and anti-Flag (IP#2) immunoprecipitates were subjected to immunoblotting with the indicated antibodies;

FIGS. 12A to 12C show that BOC and GAS1 expression do not change the cell surface localization of PTCH1 in mammalian cells. COS7 (FIG. 12A) or NIH 3T3 (FIG. 12B) cells transfected with PTCH1-GFP, BOC-Flag and GAS1 as indicated were fixed and immunolabeled without permeabilization with either anti-BOC or anti-GAS1 antibodies that recognize extracellular epitopes of BOC and GAS1. PTCH1 localization was visualized via GFP. Co-expression of BOC or GAS1 with PTCH1-GFP does not lead to a cell surface relocalization of PTCH1 in COS7 cells. FIG. 12C: COS7 cells transfected with PTCH1-GFP, BOC-Flag and GAS1 as indicated were labeled by surface biotinylation. Immunoblots showing input levels of PTCH1-GFP (top panel), biotin-labeled proteins recovered by Streptavidin agarose beads (middle panel) and actin levels (bottom panel) as a loading control.

FIG. 13 is a table summarizing the dissociation constants (Kd) calculated for the Shh mutants used in this study. Mutation E90A does not significantly affect the Kd for PTCH1 binding (7.00±1.29 nM for ShhN WT versus 5.02±0.84 nM for ShhN E90A; Student's t-test p>0.05) but abrogates binding (Kd>100 μM) to BOC, CDON, and GAS1. Mutation R154E abrogates binding to PTCH1, BOC, CDON, and GAS1;

FIG. 14 shows the effect of the FNIIIab domain of BOC on Shh signaling. C3H 10T½ cells stably transfected with a Gli-luciferase reporter were incubated in presence of N-terminal recombinant Shh (nShh) and either Fc, IgCAM-Fc, or FNIIIab-Fc. After incubation, cells were lysed and luciferase activity (an indication of the Shh signaling pathway activity) was measured. Fc: Fc fragment alone; IgCAM-Fc: IgCAM domains of BOC fused to Fc; FNIIIab-Fc. FNIIIab domains of BOC fused to Fc;

FIG. 15 shows the percentages of identity between the different domains of human BOC and human CDON (hCDO);

FIGS. 16A and 16B show the nucleotide sequence of human BOC (SEQ ID NO: 1), with the coding sequence residues (340-3684) indicated in bold, and FIG. 16C shows the amino acid sequence of human BOC (SEQ ID NO: 2);

FIGS. 17A to 17C show the nucleotide sequence of human CDON (SEQ ID NO: 3), with the coding sequence (residues 129 to 3992) indicated in bold, and FIG. 17D shows the amino acid sequence of human CDON (SEQ ID NO: 4);

FIG. 18A show the nucleotide sequence of human GAS1 (SEQ ID NO: 5), with the coding sequence (residues 411 to 1448) indicated in bold, and FIG. 18B shows the amino acid sequence of human GAS1 (SEQ ID NO: 6);

FIGS. 19A to 19C show the nucleotide sequence of human PTCH1, isoform L (SEQ ID NO: 7), with the coding sequence (residues 189 to 4532) indicated in bold, and FIG. 19D shows the amino acid sequence of human CDON (SEQ ID NO: 8);

FIG. 20 shows the nucleotide and amino acid sequences of the FNIIIab-Fc construct used in the experiments described herein;

FIG. 21 shows the nucleotide and amino acid sequences of the IgCAM-Fc construct used in the experiments described herein.

### DISCLOSURE OF INVENTION

In the studies described herein, the present inventors have demonstrated that GAS1 and BOC interact with PTCH1, and that in the absence of GAS1 and BOC, Shh-dependent proliferation is completely lost in CGNPs. They have also demonstrated that blocking the interaction between BOC and Ptch1 using a fusion protein comprising the FNIIIab domains of BOC fused to an Fc domain inhibits Shh signaling.

In a first aspect, the present invention provides a method (in vitro or in vivo) for inhibiting Sonic hedgehog (Shh)-mediated signalling in a cell, said method comprising contacting said cell with an agent that inhibits the binding of GAS1, BOC and/or CDON to

PTCH1, i.e. the interaction between (1) GAS1, BOC and/or CDON and (2) PTCH1, in said cell.

In another aspect, the present invention provides a method (in vitro or in vivo) for inhibiting Shh-mediated proliferation of a cell, said method comprising contacting said cell with an agent that inhibits the binding of GAS1, BOC and/or CDON to PTCH1 in said cell.

In another aspect, the present invention provides a method for modulating (decreasing or increasing) Shh-mediated signalling in a cell, said method comprising modulating the binding of GAS1, BOC and/or CDON to PTCH1, in said cell.

In another aspect, the present invention provides a method for modulating Shh-mediated proliferation of a cell, said method comprising modulating the binding of GAS1, BOC and/or CDON to PTCH1 in said cell.

In an embodiment, the above-mentioned cell is a neural cell.

In an embodiment, the above-mentioned said neural cell is a neuron progenitor.

In an embodiment, the above-mentioned said neural cell is a neuron progenitor is a cerebellar granular neuron progenitor (CGNP).

In another aspect, the present invention provides a method for preventing and/or treating cancer in a subject in need thereof, said method comprising administering to said subject an effective amount of an agent that inhibits the binding of agent that inhibits the binding of GAS1, BOC and/or CDON to PTCH1.

In another aspect, the present invention provides the use of an agent that inhibits the binding of GAS1, BOC and/or CDON to PTCH1 for preventing and/or treating cancer in a subject.

In another aspect, the present invention provides the use of an agent that inhibits the binding of GAS1, BOC and/or CDON to PTCH1 for the preparation of a medicament for preventing and/or treating cancer in a subject.

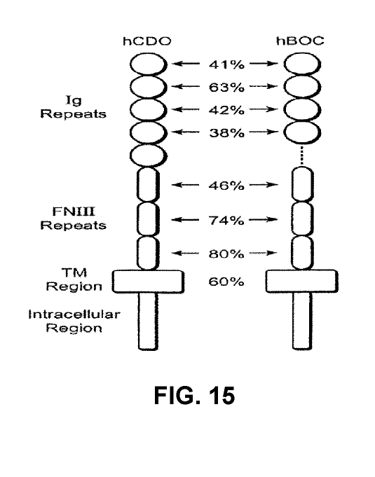

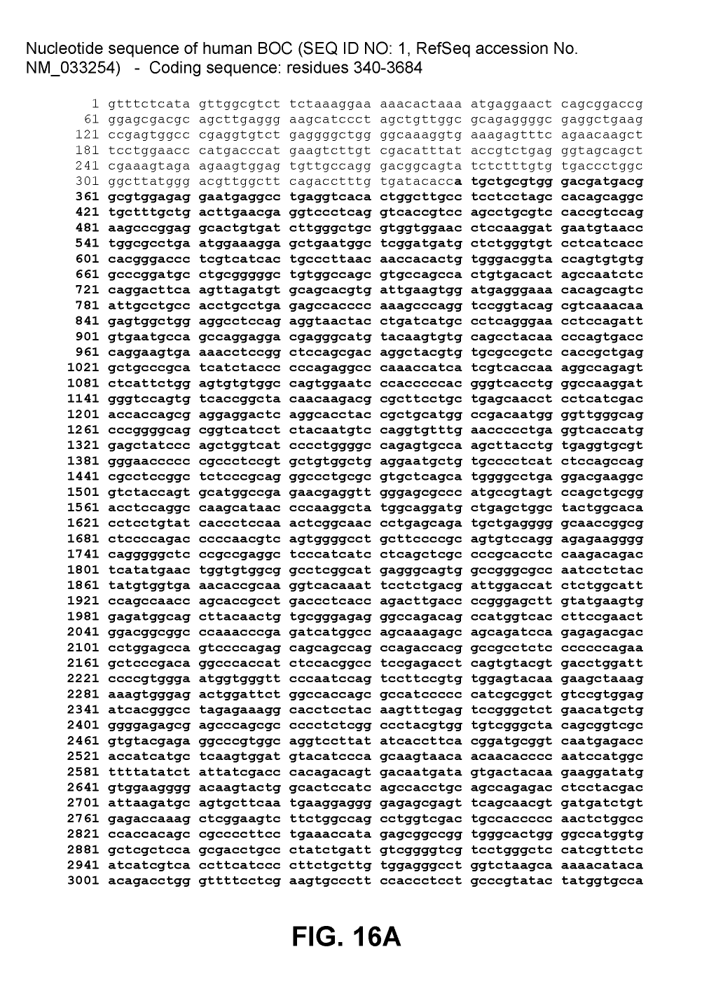

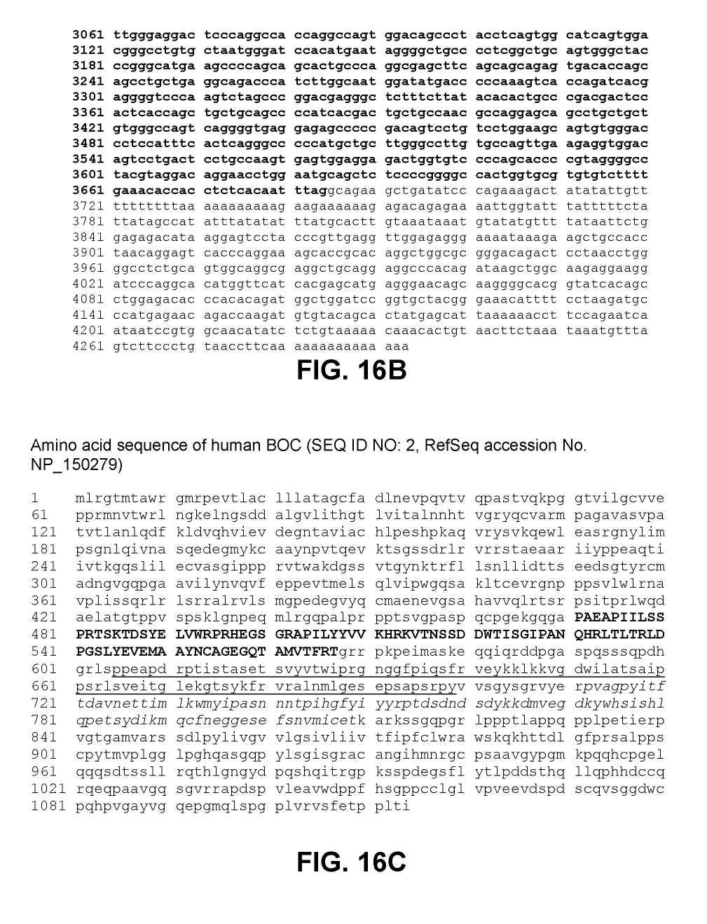

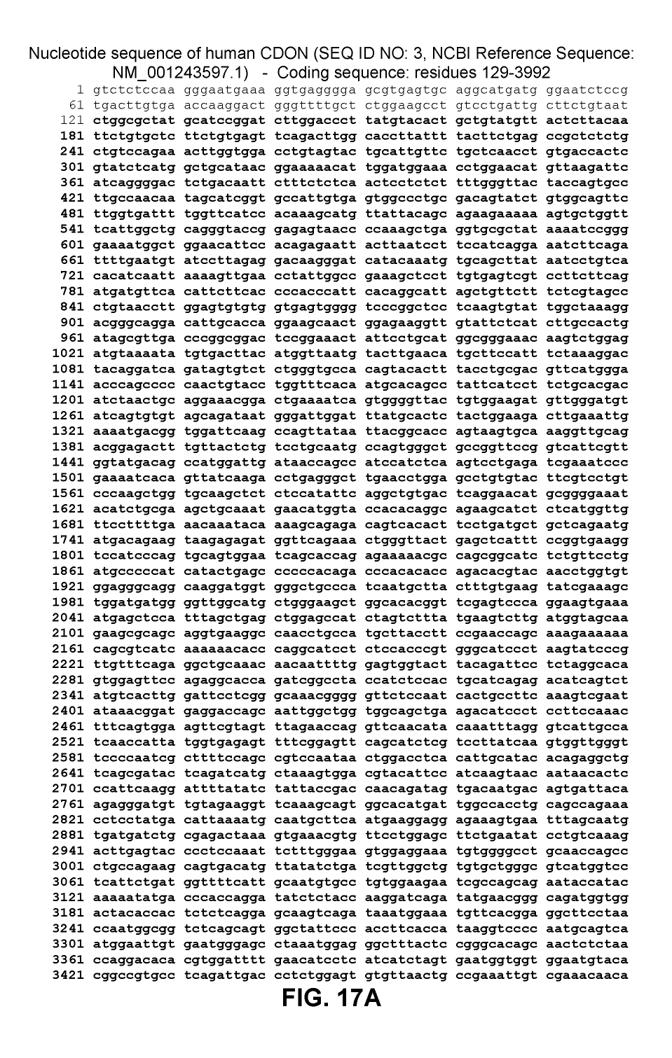

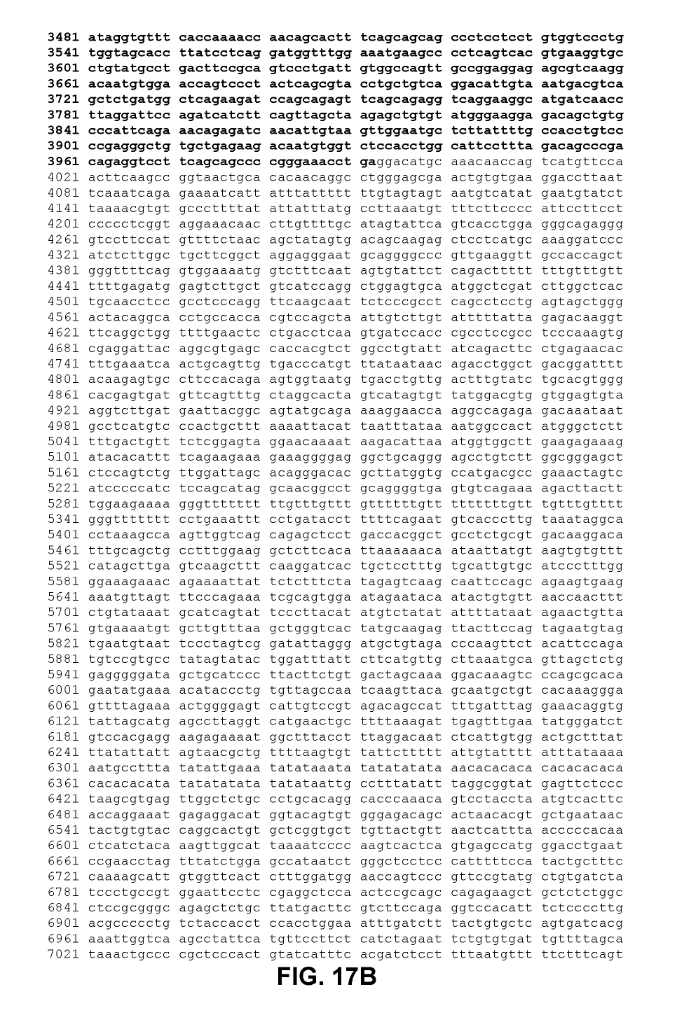

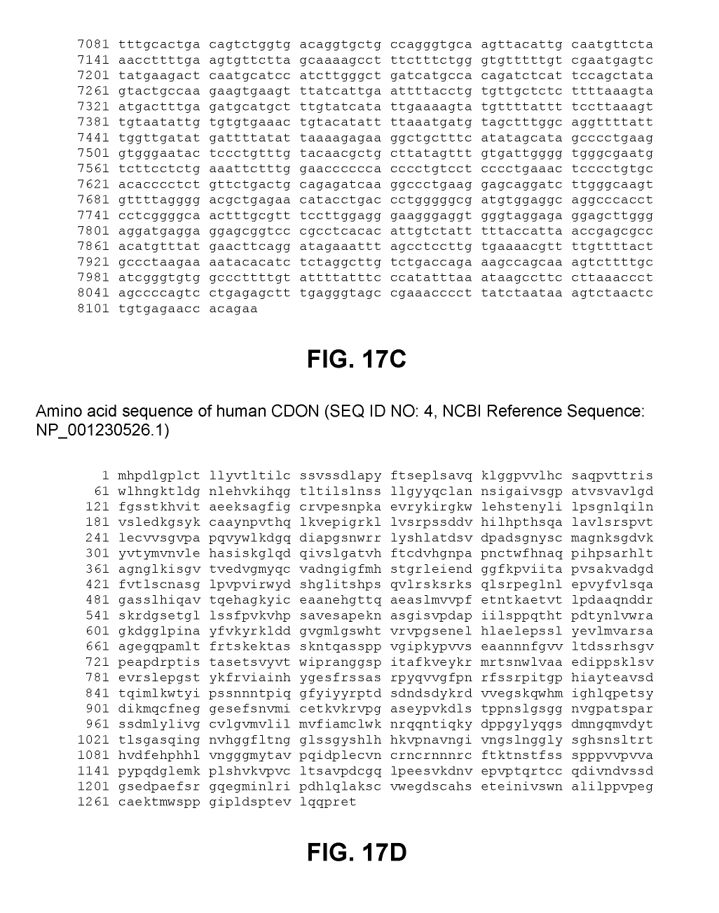

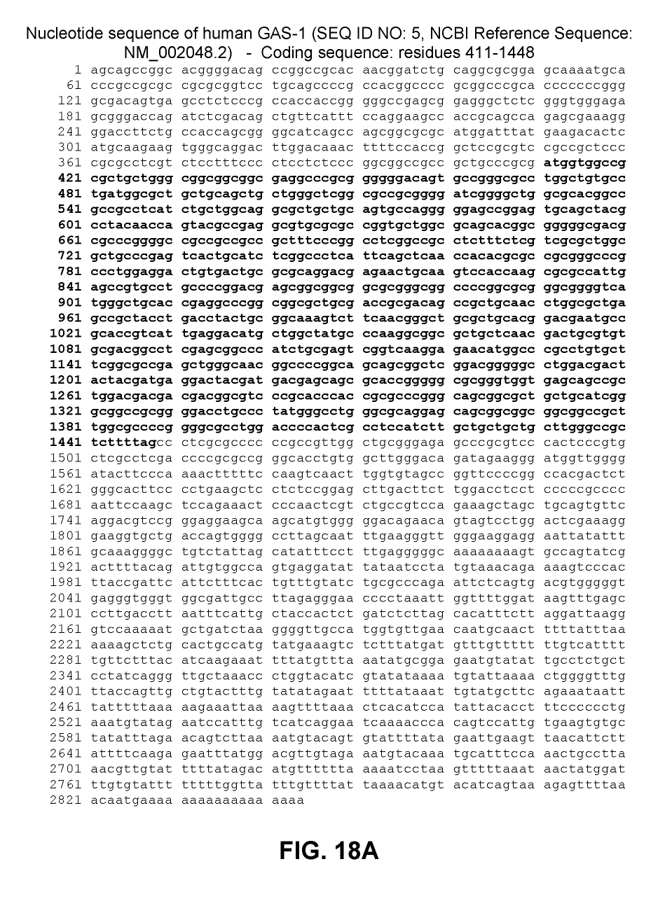

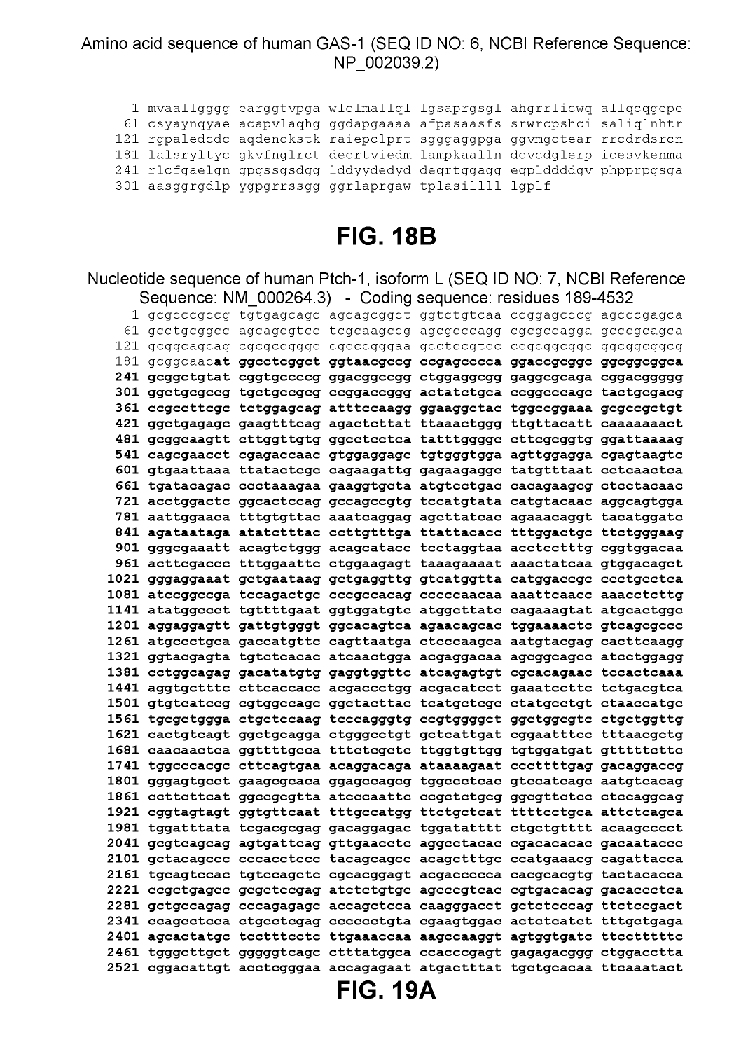

CDON and BOC are type I transmembrane receptors consisting of four or five immunoglobulin (Ig) and two or three fibronectin type III (FNIII) repeats in the extracellular domain, and an intracellular domain with no identifiable motifs. This domain architecture is closely related to that of axon guidance receptors of the Robo and DCC (deleted in colorectal cancer) families (FIG. 15). Both CDON and BOC share a high degree of homology in their extracellular domains and are expressed during early stages of development of the central nervous system (Okada et al., 2006. Nature, 444: 369-373). CDON and BOC form complexes with each other in a cis fashion.

Human growth arrest-specific 1 (GAS1) is a 345 amino acid protein comprising a putative signal peptide (residues 1-39), a mature polypeptide (residues 40-318) as well as a putative propeptide (residues 319-345) that is removed in the mature form. GAS1 is believed to be anchored to the membrane via a GPI-anchor amidated serine (residue 318). The mature polypeptide comprises two GDNF/GAS1 domains (residues 48-141 and 166-241). GDNF/GAS1 domain is a cysteine rich domain found in multiple copies in GNDF and GAS1 proteins. GDNF and neurturin (NTN) receptors are potent survival factors for sympathetic, sensory and central nervous system neurons. GDNF and neurturin promote neuronal survival by signaling through similar multicomponent receptors that consist of a common receptor tyrosine kinase and a member of a GPI-linked family of receptors that determines ligand specificity.

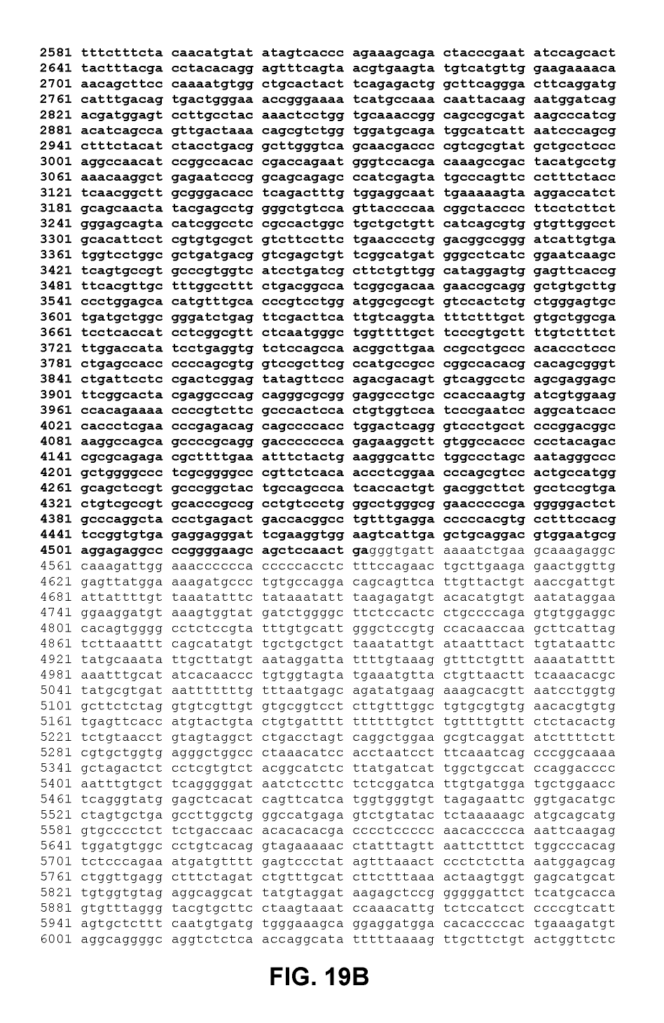

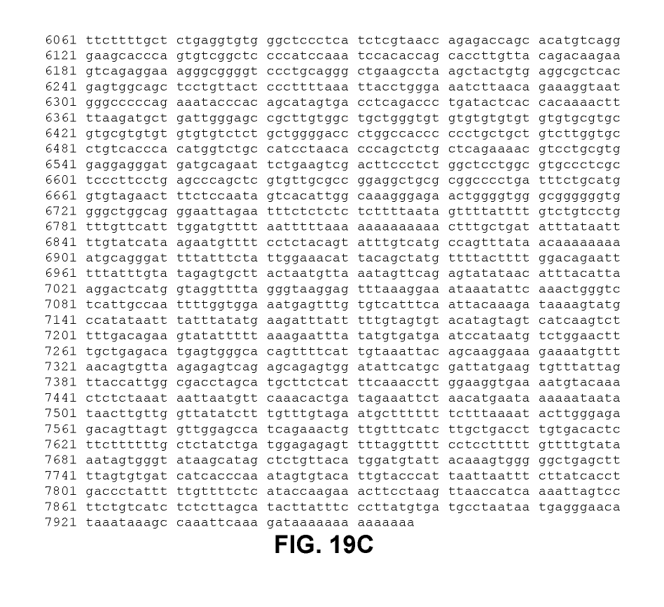

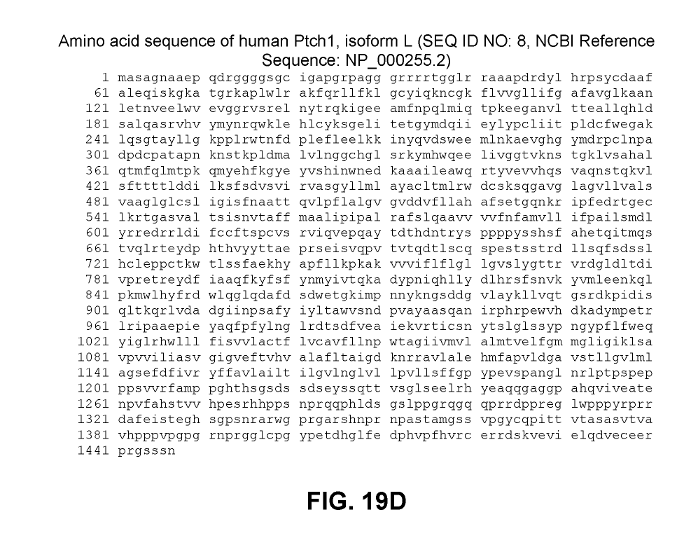

Protein patched homolog 1 (Ptch-1) is a twelve transmembrane domain protein that acts as a receptor for sonic hedgehog (SHH), indian hedgehog (IHH) and desert hedgehog (DHH). It is known to associate with the smoothened protein (SMO) to transduce the hedgehog's proteins signal. The 12 transmembrane domains are located at residues corresponding to about residues 101-121, 437-457, 473-493, 502-522, 548-568, 578-598, 749-769, 1028-1048, 1056-1076, 1084-1104, 1122-1141 and 1155-1175 in the sequence of isoform L depicted at FIG. 19D. The 6 extracellular domains are located at residues corresponding to about residues 122-436, 494-501, 569-577, 770-1027, 1077-1083 and 1142-1154 in the sequence of isoform L depicted at FIG. 19D. The 6 cytoplasmic domains are located at residues corresponding to about residues 458-472, 523-547, 599-748, 1049-1055, 1105-1121 and 1176-1447 in the sequence of isoform L depicted at FIG. 19D. Residues 438 to 598 correspond to a sterol-sensing domain (SSD). 3 others isoforms of Ptch1, produced by alternative splicing, are known (isoforms L′, M and S). The sequence of isoform L′ differs from the sequence of isoform

L as follows: residues 1-66: MASAGNAAEP . . . DAAFALEQIS→MELLNRNRLV . . . DRGDKETRSD. Isoform M of Ptch1 lacks the first 67 amino acids, and isoform S lacks the first 152 amino acids.

As used herein, the term “agent that inhibits the binding of GAS1, BOC and/or CDON to PTCH1” includes any compound or molecule (e.g., small molecules, peptides/polypeptides, antibody, non-antibody scaffolds) able to directly or indirectly affect the binding of GAS1, BOC and/or CDON to PTCH1. In an embodiment, the agent blocks the binding of GAS1 to PTCH1. In another embodiment, the agent blocks the binding of BOC to PTCH1. In another embodiment, the agent blocks the binding of CDON to PTCH1. In another embodiment, the agent blocks the binding of GAS1 and BOC to PTCH1. In another embodiment, the inhibitor blocks the binding of BOC and CDON to PTCH1. In another embodiment, the inhibitor blocks the binding of GAS1, BOC and CDON to PTCH1.

In an embodiment, the agent that inhibits the binding of BOC, CDON and/or GAS1 to PTCH1 is a polypeptide or polypeptide analog, or a nucleic acid encoding same. Such a polypeptide comprises a sequence derived from GAS1, BOC, CDON, and/or PTCH1 polypeptides, or variants thereof (e.g., polypeptide analogs), capable of inhibiting the binding of GAS1, BOC and/or CDON to PTCH1. In an embodiment, the polypeptide is a natural or synthetic contiguous amino acid sequence from the sequence of the GAS1, BOC, CDON, and/or PTCH1 polypeptides. Such polypeptides or polypeptide analogs typically bind the interacting partner or ligand of the native protein (compete for binding to its interacting partner), but without resulting in the outcome (e.g., inducing or transmitting a signal) normally associated with the interaction between the native protein and its interacting partner.

In an embodiment, the polypeptide comprises a sequence that is derived from, or corresponds to, a sequence located within the extracellular domain (ectodomain) of BOC and/or CDON.

In an embodiment, the polypeptide comprises a sequence that is derived from, or corresponds to, a sequence located within fibronectin type III domain a and/or b of BOC and/or CDON (FNIIIa and/or FNIIIb). In an embodiment, the polypeptide comprises a sequence that corresponds to a sequence located within FNIIIa. In an embodiment, the polypeptide comprises a sequence that corresponds to a sequence located within FNIIIb. In an embodiment, the polypeptide comprises a sequence that corresponds to a sequence located within FNIIIa and FNIIIb.

The FNIIIa domain of BOC corresponds to about residue 460 to about residue 570, for example from about residue 471 to residue 567 or from about residue 486 to 560 of human BOC (SEQ ID NO:2).

The FNIIIa domain of CDON corresponds to about residue 575 to about residue 675, for example from about residue 577 to about residue 673 of human CDON (SEQ ID NO:4).

The FNIIIb domain of BOC correspond to about residue 600 to about residue 700, for example from about residue 605 to residue 699 (UniProt) or from about residue 607 to 698 (NCBI) of human BOC (SEQ ID NO:2).

The FNIIIb domain of CDON correspond to about residue 720 to about residue 815, for example from about residue 721 to about residue 811 or 813 of human CDON (SEQ ID NO:4).

In an embodiment, the polypeptide comprises a sequence corresponding to a sequence located in the region spanning residue 460 to residue 570 (e.g., residue 471 to residue 567 or residue 486 to residue 560) of human BOC. In an embodiment, the polypeptide comprises a sequence corresponding to a sequence located in the region spanning residue 600 to residue 700 (e.g., residue 605 to residue 699 or residue 607 to residue 698) of human BOC. In an embodiment, the polypeptide comprises a first sequence corresponding to a sequence located in the region spanning residue 460 to residue 570 (e.g., residue 471 to residue 567 or residue 486 to residue 560) of human BOC, and a second sequence corresponding to a sequence located in the region spanning residue 600 to residue 700 (e.g., residue 605 to residue 699 or residue 607 to residue 698) of human BOC. In an embodiment, the polypeptide comprises a sequence corresponding to a sequence located in the region spanning residue 380 to residue 706 of human BOC. In another embodiment, the polypeptide comprises a sequence corresponding to a sequence located in the region spanning residue 460 to residue 700 (e.g., residue 471 to residue 698 or 699, residue 486 to residue 698 or 699) of human BOC. In a further embodiment, the polypeptide comprises a sequence corresponding to residue 460 to residue 700 (e.g., residue 471 to residue 698 or 699, residue 486 to residue 698 or 699) of human BOC.

In an embodiment, the polypeptide comprises a sequence corresponding to a sequence located in the region spanning residues 575 to about residue 675 (from about residue 577 to about residue 673) of human CDON. In an embodiment, the polypeptide comprises a sequence corresponding to a sequence located in the region spanning about residues 720 to about residue 815 (e.g., residue 721 to residue 811 or 813) of human CDON. In an embodiment, the polypeptide comprises a first sequence corresponding to a sequence located in the region spanning residues 575 to about residue 675 (from about residue 577 to about residue 673) of human CDON, and a second sequence corresponding to a sequence located in the region spanning about residue 720 to about residue 815 (e.g., residue 721 to residue 811 or 813) of human CDON. In another embodiment, the polypeptide comprises a sequence corresponding to a sequence located in the region spanning about residue 575 to about residue 815 (e.g., residue 577 to residue 811 or 813) of human CDON. In a further embodiment, the polypeptide comprises a sequence corresponding to about residue 575 to about residue 815 (e.g., residue 577 to residue 811 or 813) of human CDON.

In an embodiment, the polypeptide comprises a sequence that is derived from, or corresponds to, a sequence located within an extracellular domain of PTCH1. In an embodiment, the polypeptide comprises a sequence corresponding to a sequence located in a region spanning residue 122-436, 494-501, 569-577, 1077-1083 or 1142-1154 of human PTCH1.

In an embodiment, the polypeptide comprises a sequence corresponding to a sequence located in the region spanning residue 45 to residue 317 of human GAS1.

The expression “polypeptide comprising a sequence that is derived from another sequence” (or polypeptide analog) refers to a polypeptide comprising a sequence that is based on a native sequence (from BOC, GAS1, CDON or PTCH1) but in which at least one amino acids has been substituted or modified (e.g., an L-amino acid substituted with the corresponding D-amino acid), and that retains most or all the properties of the native polypeptide (e.g., capacity to block the interaction between GAS1, BOC and/or CDON, and PTCH1).

In embodiments, the polypeptide analogs include polypeptides with altered sequences containing substitutions of functionally equivalent amino acid residues, relative to the above-mentioned polypeptides. For example, one or more amino acid residues within the sequence can be substituted by another amino acid of a similar polarity (having similar physico-chemical properties) which acts as a functional equivalent, resulting in a silent alteration. Substitution for an amino acid within the sequence may be selected from other members of the class to which the amino acid belongs. For example, positively charged (basic) amino acids include arginine, lysine and histidine (as well as homoarginine and ornithine). Nonpolar (hydrophobic) amino acids include leucine, isoleucine, alanine, phenylalanine, valine, proline, tryptophan and methionine. Uncharged polar amino acids include serine, threonine, cysteine, tyrosine, asparagine and glutamine. Negatively charged (acidic) amino acids include glutamic acid and aspartic acid. The amino acid glycine may be included in either the nonpolar amino acid family or the uncharged (neutral) polar amino acid family. Substitutions made within a family of amino acids are generally understood to be conservative substitutions. The above-mentioned polypeptide or polypeptide analogs may comprise all L-amino acids, all D-amino acids or a mixture of L- and D-amino acids. In an embodiment, the above-mentioned polypeptide analogs comprise at least one D-amino acid (e.g., 1, 2, 3, 4, 5 or more D-amino acids).

In embodiments, the above-mentioned polypeptide or polypeptide analog is in the form of a salt, e.g., a pharmaceutically acceptable salt. As used herein the term “pharmaceutically acceptable salt” refers to salts of compounds that retain the biological activity of the parent compound, and which are not biologically or otherwise undesirable. Such salts can be prepared in situ during the final isolation and purification of the polypeptide or polypeptide analog, or may be prepared separately by reacting a free base function with a suitable acid.

Pharmaceutically acceptable acid addition salts may be prepared from inorganic and organic acids. Representative acid addition salts include, but are not limited to acetate, adipate, alginate, citrate, aspartate, benzoate, benzenesulfonate, bisulfate, butyrate, camphorate, camphor sulfonate, decanoate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, octanoate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate, and undecanoate. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Examples of acids which can be employed to form pharmaceutically acceptable acid addition salts include, for example, an inorganic acid, e.g., hydrochloric acid, hydrobromic acid, sulphuric acid, and phosphoric acid, and an organic acid, e.g., oxalic acid, maleic acid, succinic acid, and citric acid.

Basic addition salts also can be prepared by reacting a carboxylic acid-containing moiety with a suitable base such as the hydroxide, carbonate, or bicarbonate of a pharmaceutically acceptable metal cation or with ammonia or an organic primary, secondary, or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylammonium, dimethylammonium, trimethylammonium, triethylammonium, diethylammonium, and ethylammonium, amongst others. Other representative organic amines useful for the formation of base addition salts include, for example, ethylenediamine, ethanolamine, diethanolamine, piperidine, piperazine, and the like. Salts derived from organic bases include, but are not limited to, salts of primary, secondary and tertiary amines.

In an embodiment, the above-mentioned polypeptide analog is a peptidomimetic. A peptidomimetic is typically characterised by retaining the polarity, three dimensional size and functionality (bioactivity, e.g., the capacity to block the interaction between GAS1, BOC and/or

CDON, and PTCH1) of its polypeptide equivalent, but wherein one or more of the polypeptide bonds/linkages have been replaced, often by more stable linkages. Generally, the bond which replaces the amide bond (amide bond surrogate) conserves many or all of the properties of the amide bond, e.g. conformation, steric bulk, electrostatic character, potential for hydrogen bonding, etc. Typical peptide bond replacements include esters, polyamines and derivatives thereof as well as substituted alkanes and alkenes, such as aminomethyl and ketomethylene. For example, the above-mentioned polypeptide analog may have one or more peptide linkages replaced by linkages such as —CH₂NH—, —CH₂S—, —CH₂—CH₂—, —CH═CH— (cis or trans), —CH₂SO—, —CH(OH)CH₂—, or —COCH₂—. Such peptidomimetics may have greater chemical stability, enhanced biological/pharmacological properties (e.g., half-life, absorption, potency, efficiency, etc.) and/or reduced antigenicity relative its peptide equivalent.

In an embodiment, the above-mentioned polypeptide or polypeptide analog has an identity or similarity of at least 60% with a native sequence of GAS1, BOC, CDON and/or PTCH1 and retains the capacity to block the interaction between GAS1, BOC and/or CDON, and PTCH1. In further embodiments, the variant has a similarity or identity of at least 65, 70, 75, 80, 85, 90, 91, 92, 93, 94, 95, 96, 97, 98 or 99% with a native sequence of GAS1, BOC, CDON and/or PTCH1, and the capacity to block the interaction between GAS1, BOC and/or CDON, and PTCH1.

“Similarity” and “identity” refers to sequence similarity between two polypeptide molecules. The similarity or identity can be determined by comparing each position in the aligned sequences. A degree of similarity or identity between amino acid sequences is a function of the number of matching or identical amino acids at positions shared by the sequences. As the term is used herein, an amino acid sequence is “similar” or “identical” to another sequence if the two sequences are substantially similar or identical (shares at least 60% similarity or identity) and the functional activity of the sequences is conserved. As used herein, a given percentage of similarity or identity between sequences denotes the degree of sequence identity in optimally aligned sequences. An “unrelated” or “non-homologous” sequence shares less than 40% identity, though preferably less than about 25% identity, with a sequence described herein.

Optimal alignment of sequences for comparisons of similarity or identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48: 443, the search for similarity method of Pearson and Lipman, 1988, Proc. Natl. Acad. Sci. USA 85: 2444, and the computerized implementations of these algorithms (such as GAP, BESTFIT, FASTA and TFASTA in the Wisconsin Genetics Software Package, Genetics Computer Group, Madison, Wis., U.S.A.). Sequence similarity or identity may also be determined using the BLAST algorithm, described in Altschul et al., 1990, J. Mol. Biol. 215: 403-10 (using the published default settings). Software for performing BLAST analysis may be available through the National Center for Biotechnology Information web site (http://www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighborhood word score threshold. Initial neighborhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (W) of 11, the BLOSUM62 scoring matrix (Henikoff and Henikoff, 1992, Proc. Natl. Acad. Sci. USA 89: 10915-10919) alignments (B) of 50, expectation (E) of 10 (or 1 or 0.1 or 0.01 or 0.001 or 0.0001), M=5, N=4, and a comparison of both strands. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

In an embodiment, the polypeptide comprises from about 5 to about 400 amino acids, in a further embodiment from about 5, 6, 7, 8, 9 or 10 to about 250, 260, 270, 280, 290, 300, 310, 320, 330, 340, 350, 360, 370, 380, 390 or 400 amino acids, for example from about 10, 15, 20, 25, 30, 35, 40, 45, 50, 60, 70, 80, 90 or 100 to about 110, 120, 130, 140, 150, 160, 170, 180, 190, 200, 210, 220, 230 or 240 amino acids. In an embodiment, the polypeptide comprises 10, 15, 20, 25, 30, 35, 40, 45, 50, 60, 70, 80, 90 100, 110, 120, 130, 140, 150, 160, 170, 180, 190, 200, 210, 220, 230 or 240 amino acids.

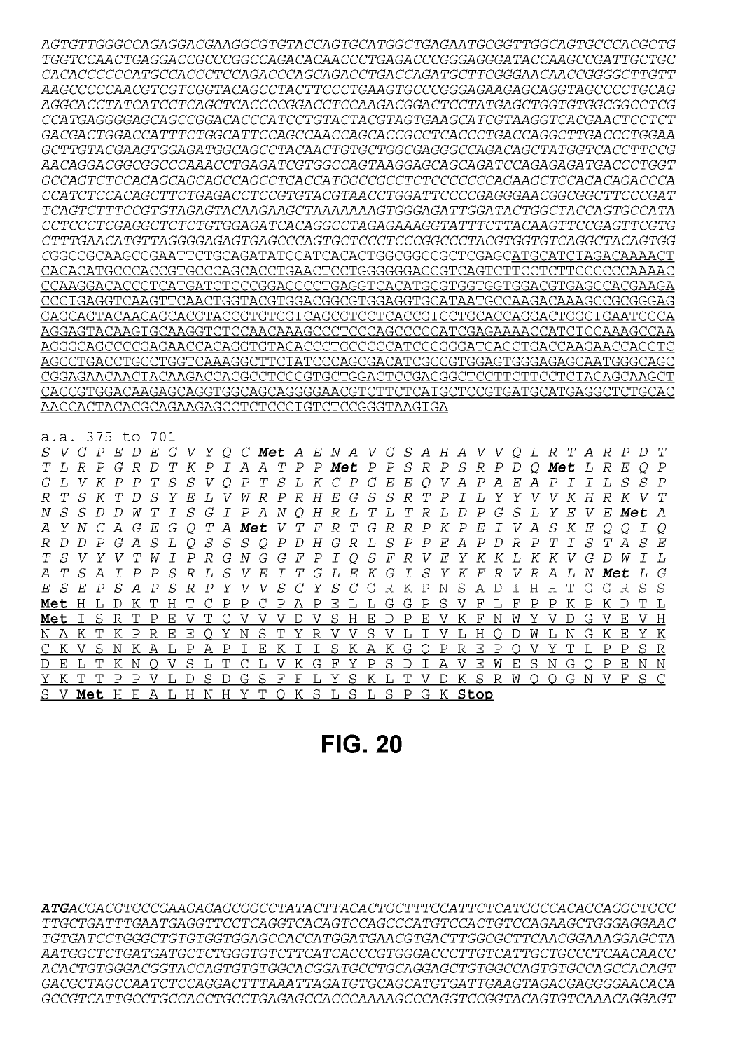

In embodiments, the above-mentioned polypeptide or polypeptide analog may comprise, further to the sequences defined above, one more amino acids (naturally occurring or synthetic) covalently linked to the amino- and/or carboxy-termini of said polypeptide or polypeptide analog. Accordingly, in an embodiment, the above-mentioned polypeptide or polypeptide analog is a fusion polypeptide comprising a first domain comprising the sequences defined above (i.e., sequences derived from GAS1, BOC, CDON, and/or PTCH1 polypeptides, fragments or variants thereof) and a second domain. In an embodiment, the second domain is N-terminal relative to the first domain. In another embodiment, the second domain is C-terminal relative to the first domain. In an embodiment, the second domain is an Fc domain. In an embodiment, the agent is the FNIIIab-Fc construct of FIG. 20 (SEQ ID NO: 10).

In an embodiment, the amino terminal residue (i.e., the free amino group at the N-terminal end) of the polypeptide or polypeptide analog is modified (e.g., for protection against degradation), for example by covalent attachment of a moiety/chemical group. In an embodiment, the amino-terminal modification is a C₁-C₁₆ or C₃-C₁₆ acyl group (linear or branched, saturated or unsaturated), in a further embodiment, a saturated C₁-C₆ acyl group (linear or branched) or an unsaturated C₃-C₆ acyl group (linear or branched), in a further embodiment an acetyl group (CH₃—CO—, Ac).

In an embodiment, the carboxy terminal residue (i.e., the free carboxy group at the C-terminal end of the polypeptide) of the polypeptide or polypeptide analog is modified (e.g., for protection against degradation). In an embodiment, the modification is an amidation (replacement of the OH group by a NH₂ group).

The polypeptide or polypeptide analog may further comprise modifications that confer additional biological properties to the polypeptide or polypeptide analog such as protease resistance, plasma protein binding, increased plasma half-life, intracellular penetration, etc. Such modifications include, for example, covalent attachment of fatty acids (e.g., C₆-C₁₈), attachment to proteins such as albumin (see, e.g., U.S. Pat. No. 7,268,113); glycosylation, biotinylation or PEGylation (see, e.g., U.S. Pat. Nos. 7,256,258 and 6,528,485). The above description of modification of the polypeptide or polypeptide analog does not limit the scope of the approaches nor the possible modifications that can be engineered.

A polypeptide may be prepared by conventional synthetic methods or recombinant DNA technologies. The methods for synthetic production of peptides and polypeptides are well known in art. Suitable syntheses can be performed for example by utilizing “T-boc” or “Fmoc” procedures. Techniques and procedures for solid phase synthesis are described in for example Solid Phase Peptide Synthesis: A Practical Approach, by E. Atherton and R. C. Sheppard, published by IRL, Oxford University Press, 1989. Alternatively, the peptides may be prepared by way of segment condensation, as described, for example, in Liu et al., Tetrahedron Lett. 37: 933-936, 1996; Baca et al., J. Am. Chem. Soc. 117: 1881-1887, 1995; Tam et al., Int. J. Peptide Protein Res. 45: 209-216, 1995; Schnolzer and Kent, Science 256: 221-225, 1992; Liu and Tam, J. Am. Chem. Soc. 116: 4149-4153, 1994; Liu and Tam, Proc. Natl. Acad. Sci. USA 91: 6584-6588, 1994; and Yamashiro and Li, Int. J. Peptide Protein Res. 31: 322-334, 1988). Other methods useful for synthesizing the peptides are described in Nakagawa et al., J. Am. Chem. Soc. 107: 7087-7092, 1985. Detailed descriptions as well as practical advice for producing synthetic peptides may be found in Synthetic Peptides: A Users Guide (Advances in Molecular Biology), Grant G. A. ed., Oxford University Press, 2002, or in Pharmaceutical Formulation: Development of Peptides and Proteins, Frokjaer and Hovgaard eds., Taylor and Francis, 1999.

Polypeptides and polypeptide analogs comprising naturally occurring amino acids encoded by the genetic code may also be prepared using recombinant DNA technology using standard methods. Polypeptides produced by recombinant technology may be modified (e.g., N-terminal acylation [e.g., acetylation], C-terminal amidation), using methods well known in the art. Therefore, in embodiments, in cases where a polypeptide or polypeptide analog described herein contains naturally occurring amino acids encoded by the genetic code, the polypeptide or polypeptide analog may be produced using recombinant methods, and may in embodiments be subjected to for example the just-noted modifications (e.g., acylation, amidation).

Accordingly, in another aspect, the above-mentioned agent that inhibits the binding of GAS1, BOC and/or CDON to PTCH1 is a nucleic acid encoding the above-mentioned polypeptide or polypeptide analog. The invention also provides a vector comprising the above-mentioned nucleic acid. In yet another aspect, the present invention provides a cell (e.g., a host cell) comprising the above-mentioned nucleic acid and/or vector. Accordingly, the above-mentioned methods or uses encompass gene therapy or cell therapy techniques for indirectly delivering the above-mentioned polypeptide or polypeptide analog to a subject in need thereof.

The invention further provides a recombinant expression system, vectors and host cells, such as those described above, for the expression/production of a polypeptide or polypeptide analog, using for example culture media, production, isolation and purification methods well known in the art.

In an embodiment, the agent that inhibits the binding of GAS1, BOC and/or CDON to PTCH1 is an antibody or an antigen-binding fragment thereof.

The term antibody or immunoglobulin is used in the broadest sense, and covers monoclonal antibodies (including full-length monoclonal antibodies), polyclonal antibodies, multispecific antibodies, chimeric antibodies and humanized antibodies, so long as they exhibit the desired biological activity (inhibiting the binding of GAS1, BOC and/or CDON to PTCH1). Antibody fragments comprise a portion of a full length antibody, generally an antigen binding or variable region thereof. Examples of antibody fragments include Fab, Fab′, F(ab′)2, and Fv fragments, diabodies, linear antibodies, single-chain antibody molecules, single domain antibodies (e.g., from camelids), shark NAR single domain antibodies, and multispecific antibodies formed from antibody fragments. Antibody fragments can also refer to binding moieties comprising CDRs or antigen binding domains including, but not limited to, V_{H} regions (V_{H}, V_{H}-V_{H}), anticalins, PepBodies, antibody-T-cell epitope fusions (Troybodies) or Peptibodies.

In an embodiment, the antibody or antibody fragment is directed against (or specific for) a sequence (epitope) derived from a GAS1, BOC, CDON, and/or PTCH1 polypeptide. In an embodiment, the antibody or antibody fragment is directed against a sequence that is derived from, or correspond to, a sequence located within fibronectin type III domain a and/or b of BOC and/or CDON (FNIIIa and/or FNIIIb). In an embodiment, the antibody or antibody fragment is directed against a sequence located within FNIIIa. In an embodiment, the antibody or antibody fragment is directed against a sequence located within FNIIIb. In an embodiment, the antibody or antibody fragment is directed against a sequence located within FNIIIa and FNIIIb. In an embodiment, the antibody or antibody fragment is directed against a sequence located in the region spanning residues 460 to residue 570 (e.g., residue 471 to residue 567 or residue 486 to residue 560) of human BOC. In an embodiment, the antibody or antibody fragment is directed against a sequence located in the region spanning residues 600 to residue 700 (e.g., residue 605 to residue 699 or residue 607 to residue 698) of human BOC. In an embodiment, the antibody or antibody fragment is directed against a first sequence located in the region spanning residues 460 to residue 570 (e.g., residue 471 to residue 567 or residue 486 to residue 560) of human BOC, and a second sequence located in the region spanning residue 600 to residue 700 (e.g., residue 605 to residue 699 or residue 607 to residue 698) of human BOC.

In an embodiment, the antibody or antibody fragment is directed against a sequence located in the region spanning residues 575 to about residue 675 (from about residue 577 to about residue 673) of human CDON. In an embodiment, the antibody or antibody fragment is directed against a sequence located in the region spanning about residues 720 to about residue 815 (e.g., residue 721 to residue 811 or 813) of human CDON. In an embodiment, the antibody or antibody fragment is directed against a first sequence located in the region spanning residues 575 to about residue 675 (from about residue 577 to about residue 673) of human CDON, and a second sequence located in the region spanning about residue 720 to about residue 815 (e.g., residue 721 to residue 811 or 813) of human CDON.

In an embodiment, the antibody or antibody fragment is directed against a sequence located in an extracellular domain of PTCH1. In an embodiment, the antibody or antibody fragment is directed against a sequence located in a region spanning residue 122-436, 494-501, 569-577, 1077-1083 or 1142-1154 of human PTCH1.

In an embodiment, the antibody or antibody fragment is directed against a sequence located in the region spanning residue 45 to residue 317 of human GAS1.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody A Laboratory Manual, CSH Laboratories).

Polyclonal antibodies are preferably raised in animals by multiple subcutaneous (s.c.), intravenous (i.v.) or intraperitoneal (i.p.) injections of the relevant antigen (e.g., a peptide or polypeptide comprising a sequence of GAS1, BOC, CDON or PTCH1) with or without an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals may be immunized against the antigen, immunogenic conjugates, or derivatives by combining the antigen or conjugate (e.g., 100 μg for rabbits or 5 μg for mice) with 3 volumes of Freund's complete adjuvant and injecting the solution intradermally at multiple sites. One month later the animals are boosted with the antigen or conjugate (e.g., with ⅕ to 1/10 of the original amount used to immunize) in Freund's complete adjuvant by subcutaneous injection at multiple sites. Seven to 14 days later the animals are bled and the serum is assayed for antibody titer. Animals are boosted until the titer plateaus. Preferably, for conjugate immunizations, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal antibodies may be made using the hybridoma method first described by Kohler et al., Nature, 256: 495 (1975), or may be made by recombinant DNA methods (e.g., U.S. Pat. No. 6,204,023). Monoclonal antibodies may also be made using the techniques described in U.S. Pat. Nos. 6,025,155 and 6,077,677 as well as U.S. Patent Application Publication Nos. 2002/0160970 and 2003/0083293.

In the hybridoma method, a mouse or other appropriate host animal, such as a rat, hamster or monkey, is immunized (e.g., as hereinabove described) to elicit lymphocytes that produce or are capable of producing antibodies that will specifically bind to the antigen used for immunization. Alternatively, lymphocytes may be immunized in vitro. Lymphocytes then are fused with myeloma cells using a suitable fusing agent, such as polyethylene glycol, to form a hybridoma cell.

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

The agent that inhibits the binding of GAS1, BOC and/or CDON to PTCH1 may also be in the form of non-antibody-based scaffolds capable of binding specific sequences, such as avimers (Avidia); DARPins (Molecular Partners); Adnectins (Adnexus), Anticalins (Pieris) and Affibodies (Affibody). The use of alternative scaffolds for protein binding is well known in the art (see, for example, Binz and Plückthun, 2005, Curr. Opin. Biotech. 16: 1-11).

In another aspect, the present invention provides a nucleic acid encoding the above-mentioned agent (e.g., polypeptide or antibody) that blocks the binding of GAS1, BOC and/or CDON to PTCH1. Such nucleic acid may be incorporated into a cell to induce the production of the polypeptide or antibody by the cell, which in turn may the binding of GAS1, BOC and/or CDON to PTCH1.

The above-mentioned agent may be formulated with one or more pharmaceutically acceptable diluent, carrier, excipient salt or adjuvant in a pharmaceutical composition. Diluents, carriers, excipients include, for example, saline, buffered saline, dextrose, water, glycerol, ethanol, and combinations thereof. The pharmaceutical composition may be adapted for the desired route of administration (e.g., oral, parental, intravenous, intramuscular, intraperitoneal, aerosol, intrathecal, intralumbar).

The present invention also provides a kit or package for inhibiting Shh-mediated signalling and/or proliferation in a cell, or for preventing and/or treating cancer, the kit comprising the above-mentioned agent or pharmaceutical composition. Such kit may further comprise, for example, instructions for inhibiting Shh-mediated signalling and/or proliferation in a cell, or for the prevention and/or treatment of cancer, containers, devices for administering the agent/composition, etc.

In an embodiment, the above-mentioned prevention/treatment comprises the use/administration of more than one (i.e. a combination of) active/therapeutic agent. The combination of prophylactic/therapeutic agents and/or compositions of the present invention may be administered or co-administered (e.g., consecutively, simultaneously, at different times) in any conventional dosage form. Co-administration in the context of the present invention refers to the administration of more than one prophylactic or therapeutic agent in the course of a coordinated treatment to achieve an improved clinical outcome. Such co-administration may also be coextensive, that is, occurring during overlapping periods of time. For example, a first agent may be administered to a patient before, concomitantly, before and after, or after a second active agent is administered. The agents may in an embodiment be combined/formulated in a single composition and thus administered at the same time. In an embodiment, the one or more active agent(s) of the present invention is used/administered in combination with one or more agent(s) currently used to prevent or treat the disorder in question (e.g., an anticancer agent). In an embodiment, the agent that inhibits the binding of GAS1, BOC and/or CDON to PTCH1 is used in combination with other anticancer treatment including chemotherapeutical agents, such as cyclopamine, CUR0199691, Cisplatin™, Oxaliplatin™ and their derivatives, cyclophosphamide compound (Cy), 13-cis retinoic acid (RA), histone deacetylase inhibitor (SAHA), nucleotide analogues (e.g., 5-fluoro uracyl, 5-AZA), kinase inhibitors, or with other inhibitors of the Shh pathway such as the SMO antagonists Cyclopamine, HhAntag691, GDC-0449, IPI-926 and LDE225, the Hh inhibitor Robotnikinin, as well as the Gli inhibitors GANT61 and JK184 (described in Peukert and Miller-Moslin, Chem Med Chem 2010, 5, 500-512). In an embodiment, the above-mentioned method or use comprises the administration or use of more than one agent as defined herein, for example a first agent blocking the binding of GAS1 to PTCH1, and a second agent blocking the binding of BOC to PTCH1.

The amount of the agent or pharmaceutical composition which is effective in the prevention and/or treatment of a particular disease, disorder or condition (e.g., cancer) will depend on the nature and severity of the disease, the chosen prophylactic/therapeutic regimen (i.e., compound, DNA construct, protein, cells), the target site of action, the patient's weight, special diets being followed by the patient, concurrent medications being used, the administration route and other factors that will be recognized by those skilled in the art. The dosage will be adapted by the clinician in accordance with conventional factors such as the extent of the disease and different parameters from the patient. Typically, 0.001 to 1000 mg/kg of body weight/day will be administered to the subject. In an embodiment, a daily dose range of about 0.01 mg/kg to about 500 mg/kg, in a further embodiment of about 0.1 mg/kg to about 200 mg/kg, in a further embodiment of about 1 mg/kg to about 100 mg/kg, in a further embodiment of about 10 mg/kg to about 50 mg/kg, may be used. The dose administered to a patient, in the context of the present invention should be sufficient to effect a beneficial prophylactic and/or therapeutic response in the patient over time. The size of the dose will also be determined by the existence, nature, and extent of any adverse side-effects that accompany the administration. Effective doses may be extrapolated from dose response curves derived from in vitro or animal model test systems. For example, in order to obtain an effective mg/kg dose for humans based on data generated from rat studies, the effective mg/kg dosage in rat may be divided by six.

The terms “treat/treating/treatment” and “prevent/preventing/prevention” as used herein, refers to eliciting the desired biological response, i.e., a therapeutic and prophylactic effect, respectively. In accordance with the subject invention, the therapeutic effect comprises one or more of a decrease/reduction in tumor, a decrease/reduction in the severity of the cancer (e.g., a reduction or inhibition of metastasis development), a decrease/reduction in symptoms and cancer-related effects, an amelioration of symptoms and cancer-related effects, and an increased survival time of the affected host animal, following administration of the agent/composition of the invention. In accordance with the invention, a prophylactic effect may comprise a complete or partial avoidance/inhibition or a delay of cancer development/progression (e.g., a complete or partial avoidance/inhibition or a delay of metastasis development), and an increased survival time of the affected host animal, following administration of the agent that inhibits binding of GAS1, BOC and/or CDON to PTCH1 (or of a composition comprising the agent).

As such, a “therapeutically effective” or “prophylactically effective” amount of an agent that inhibits the binding of GAS1, BOC and/or CDON to PTCH1, or a combination of such agents, may be administered to an animal, in the context of the methods of treatment and prevention, respectively, described herein.

As used herein, “inhibition” or “decrease” of binding of GAS1, BOC and/or CDON to PTCH1 refers to a reduction in of at least 10% as compared to reference (e.g., normal) binding in the absence of the agent, in an embodiment of at least 20% lower, in a further embodiment of at least 30%, in a further embodiment of at least 40%, in a further embodiment of at least 50%, in a further embodiment of at least 60%, in a further embodiment of at least 70%, in a further embodiment of at least 80%, in a further embodiment of at least 90%, in a further embodiment of 100% (complete inhibition).

In an embodiment, the above-mentioned agent blocks or inhibits paracrine Hedgehog signaling (i.e. by blocking the binding of GAS1, BOC and/or CDOM to PTCH1), for example by inhibiting the activation of Hedgehog signaling in the surrounding stroma by Hh ligand-secreting tumor cells. The activation of Hh signaling in the stroma surrounding the tumors has recently been shown to create a favorable environment for tumor growth (Theunissen J W and de Sauvage F J., Cancer Res. 2009, 69(15): 6007-10. Epub 2009 Jul. 28; Scales S J and de Sauvage F J. Trends Pharmacol Sci. 2009 30(6): 303-12. Epub 2009 May 13; Tian H et al., Proc Natl Acad Sci USA. 2009 106(11): 4254-9. Epub 2009 Feb. 25; Yauch R L et al., Nature. 2008, 455(7211): 406-10. Epub 2008 Aug. 27).

In another aspect, the present invention provides a method (in vitro or in vivo) of identifying an agent that may be used for inhibiting Sonic hedgehog (Shh)-mediated signalling in a cell, said method comprising determining the binding of a GAS1, BOC and/or CDON polypeptide to a PTCH1 polypeptide in the presence of said agent, wherein a lower or decreased binding in the presence of said agent is indicative that said agent may be used for inhibiting Sonic hedgehog (Shh)-mediated signalling in a cell.

In another aspect, the present invention provides a method of identifying an agent that may be used for inhibiting Sonic hedgehog (Shh)-mediated proliferation of a cell, said method comprising determining the binding of a GAS1, BOC and/or CDON polypeptide to a PTCH1 polypeptide in the presence of said agent, wherein a lower or decreased binding in the presence of said agent is indicative that said agent may be used for inhibiting Sonic hedgehog (Shh)-mediated proliferation of a cell.

In another aspect, the present invention provides a method of identifying an agent that may be used for the treatment of cancer, said method comprising determining the binding of a GAS1, BOC and/or CDON polypeptide to a PTCH1 polypeptide in the presence of said agent, wherein a lower or decreased binding in the presence of said agent is indicative that said agent may be used for the treatment of cancer.

The above-mentioned screening methods (e.g., in vitro) may be employed either with a single test compound or a plurality or library (e.g., a combinatorial library) of test compounds. In the latter case, synergistic effects provided by combinations of compounds may also be identified and characterized. The above-mentioned compounds may be used for prevention and/or treatment of cancer, or may be used as lead compounds for the development and testing of additional compounds having improved specificity, efficacy and/or pharmacological (e.g., pharmacokinetic) properties. In an embodiment the compound may be a prodrug which is altered into its active form at the appropriate site of action, (e.g., a cell, tissue or organ affected by cancer). In certain embodiments, one or a plurality of the steps of the screening/testing methods of the invention may be automated.

Such assay systems may comprise a variety of means to enable and optimize useful assay conditions. Such means may include but are not limited to: suitable buffer solutions, for example, for the control of pH and ionic strength and to provide any necessary components for optimal activity and stability (e.g., protease inhibitors), temperature control means for activity and or stability, and detection means to enable the detection of the binding of GAS1, BOC and/or CDON to PTCH1. A variety of such detection means may be used, including but not limited to one or a combination of the following: radiolabelling (e.g., ³²P, ¹⁴C, ³H), antibody-based detection, fluorescence, chemiluminescence, spectroscopic methods (e.g., generation of a product with altered spectroscopic properties), various reporter enzymes or proteins (e.g., horseradish peroxidase, green fluorescent protein), specific binding reagents (e.g., biotin/(strept)avidin), and others.

The assay may be carried out in vitro utilizing a source of GAS1, BOC, CDON and/or PTCH1, which may comprise naturally isolated or recombinantly produced GAS1, BOC, CDON, and/or PTCH1, in preparations ranging from crude to pure. Recombinant GAS1, BOC, CDON and/or PTCH1 may be produced in a number of prokaryotic or eukaryotic expression systems, which are well known in the art. Such assays may be performed in an array format.

The invention further relates to methods for the identification and characterization of compounds capable of decreasing the binding of GAS1, BOC, and/or CDON to PTCH1. For example, a test compound may be added to a reaction mixture containing a purified GAS1, BOC, and/or CDON, and a purified PTCH1 polypeptide (or a peptide fragment thereof), and the binding between GAS1, BOC, and/or CDON and PTCH1 is determined and compared to the binding when the mixture is incubated under similar conditions but without the test compound. A lower binding in the presence of the test compound is indicative that the test compound may be useful for decreasing the binding of GAS1, BOC, and/or CDON to PTCH1 (thus decreasing Shh signaling) and in turn for the prevention and/or treatment of an associated disease (e.g., cancer). The detection step (i.e. determination of the binding) could be monitored by any number of means including, but not limited to binding-dependent optical spectroscopy, fluorimetry, and radioactive label variation and could use various techniques such as surface plasmon resonance (SPR), FRET, yeast two hybrids, and alpha-screen. The above-mentioned methods may be performed in a cell-free or cell-based assay.

The activity of the agent may be also characterized using enzymatic, biochemical or cellular activity attributable to Shh signalling. Without being so limited, such activities include the activation of any subsequent step of the Hedgehog pathway including the transcription of Hh-targeted genes (e.g., Gli-1, PTC), and cell proliferation (which may be measured, for example, by an MTT assay, BrdU incorporation, carboxyfluorescein succinimidyl ester (CFSE) dilution, or Ki-67 immunodetection).

In an embodiment, the above-mentioned cancer is associated with abnormal hedgehog and/or Wnt signalling (i.e. abnormal/aberrant activation of the Hh and/or Wnt pathway(s)). In an embodiment, the above-mentioned cancer is associated with abnormal/aberrant activation of the Hh pathway, e.g., abnormal/aberrant Shh-mediated activation of the Hh pathway. Examples of cancers known to be linked to abnormal or aberrant Hh signaling include basal cell carcinoma, medulloblastoma, rhabdomyosarcoma, glioma, breast cancer, esophageal cancer, gastric cancer, pancreatic cancer, prostate cancer, small-cell lung cancer, biliary tract cancer, bladder cancer and oral cancer (see Varjosalo and Taipale, Genes and Development 2008, 22:2454-2472). Also, somatic mutations in Ptch and Smo that trigger constitutive and cell-autonomous Hh pathway activation have been found in 20% of pediatric medulloblastomas and in more than 70% of sporadic basal cell carcinoma (BCC). Additionally, tumor types such as small-cell lung cancer, as well as gastric, pancreatic, and prostate cancer, have been reported to display abnormal activation of the Hh pathway in the absence of known mutations (Peukert and Miller-Moslin, Chem Med Chem 2010, 5, 500-512).

In an embodiment, the above-mentioned cancer/tumor is associated with GAS1, BOC and/or CDON expression and/or activity (e.g., GAS1, BOC and/or CDON overexpression or increased/abnormal GAS1, BOC and/or CDON activity). Accordingly, in another aspect, the above-mentioned method or use further comprises determining whether GAS1, BOC and/or CDON is expressed or overexpressed (relative to a control), and/or if GAS1, BOC and/or CDON activity is detected or is abnormally increased (relative to a control), in said cancer. Such expression or overexpression may be determined, for example, by measuring the expression of GAS1, BOC and/or CDON (at the mRNA and/or protein level) in a biological sample (e.g., a tumor sample) from the subject.

In an embodiment, the above-mentioned cancer/tumor is associated with GAS1 expression and/or activity (e.g., GAS1 overexpression or increased/abnormal GAS1 activity).

In another embodiment, the above-mentioned cancer/tumor is associated with BOC expression and/or activity (e.g., BOC overexpression or increased/abnormal BOC activity). BOC has been shown to be overexpressed or abnormally expressed in various human tumors, including medulloblastoma tumors as well as brain ependynoma, ovary tumor, medulloblastoma, breast tumor, skin tumor, glioblastoma, meningioma, astrocytoma, chondrosarcoma, colon adenocarcinoma, liver cholangiocarcinoma, prostate carcinoma, bladder tumor, lung tumor, lymph node lymphoma, vascular endothelium hemangioma, kidney carcinoma and thyroid follicular adenoma (see, e.g., U.S. patent application Ser. No. 12/555,077)

In another embodiment, the above-mentioned cancer/tumor is associated with CDON expression and/or activity (e.g., CDON overexpression or increased/abnormal CDON activity).

In another embodiment, the above-mentioned cancer/tumor is associated with PTCH1 expression and/or activity (e.g., PTCH1 overexpression or increased/abnormal PTCH1 activity).

In another embodiment, the above-mentioned cancer is a brain tumor (e.g., brain ependynoma, a medulloblastoma), an ovary tumor (e.g., ovary carcinoma, endometriosis or adenocarcinoma), a breast tumor (e.g., breast fibroadenoma or carcinoma), a glioblastoma, a skin tumor (e.g., skin melanoma), a meningioma, an astrocytoma, a liver tumor (e.g., liver cholangiocarcinoma), a prostate carcinoma, basal cell carcinoma, gastrointestinal cancers (e.g., gastric cancer), esophageal cancer, a bladder tumor (e.g., bladder adenocarcinoma), a lung tumor, hematological malignancies (lymph node lymphoma), a vascular endothelium hemangioma, pancreatic cancer a kidney carcinoma or a thyroid follicular adenoma.

In another embodiment, the above-mentioned cancer is a cancer of the central nervous system (e.g., brain cancer). In a further embodiment, the above-mentioned cancer is a neuroectodermal tumor. In yet a further embodiment, the above-mentioned cancer is a medulloblastoma. In a further embodiment, the above-mentioned medulloblastoma is a medulloblastoma associated with a deregulation of the Hh pathway and/or of the Wnt pathway.

In another aspect, the present invention provides a method of preventing or delaying cancer development in a subject (e.g., in a subject at risk of developing cancer), said method comprising administering to said subject an effective amount of an agent that inhibits the binding of a Growth Arrest Specific 1 (GAS1) polypeptide, a cell-adhesion-molecule-related/downregulated by oncogenes (CDON) polypeptide and/or biregional CDON-binding protein (BOC) polypeptide to a Patched1 (PTCH1) polypeptide. In an embodiment, the subject has Gorlin syndrome (also known as nevoid basal cell carcinoma, BCC).

(BCC).

Gorlin syndrome patients are heterozygous for PTCH1 (similar to Ptch1^{+/-} mice) and have a high risk of developing sporadic BCC, ovarian cysts, ovarian carcinoma and medulloblastoma (Peukert and Miller-Moslin, Chem Med Chem 2010, 5, 500-512), and are expected to have pre-neoplasia that may transition to aggressive tumors. It is thus worthwhile to consider inhibiting Shh signaling at early stages in these patients, notably using the agents described herein. Therapies aimed at inhibiting the interaction of GAS1, BOC and/or CDON with PTCH1 may efficiently inhibit the formation of advanced medulloblastoma with less adverse effects than the inhibition of less specific target, such as SMO. Thus, the present invention relates to preventative cancer therapies for individuals at risk of developing cancer, such as Gorlin syndrome patients.

As used herein the term “subject” is meant to refer to any animal, such as a mammal including human, mice, rat, dog, cat, pig, cow, monkey, horse, etc. In a particular embodiment, it refers to a human.

A “subject in need thereof” or a “patient” in the context of the present invention is intended to include any subject that will benefit or that is likely to benefit from the decrease in the binding between GAS1, BOC, and/or CDON and a Shh and/or PTCH1 and its associated biological effects (e.g. decreased Shh signalling).

As used herein, the term “a” or “the” means “at least one”. Other objects, advantages and features of the present invention will become more apparent upon reading of the following non-restrictive description of specific embodiments thereof, given by way of example only with reference to the accompanying drawings.

### MODE(S) FOR CARRYING OUT THE INVENTION

The present invention is illustrated in further details by the following non-limiting examples.

### Example 1

### Materials and Methods

Mice.

Boc and Cdon (Okada et al., 2006; Zhang et al., 2010) and Gas1 (Martinelli and Fan, 2007a) mice were described previously.

Plasmids and Reagents.

Recombinant ShhN C24II and IGF-I were from R&D Systems. pEGFP-mCdon, pEGFP-mBoc, and pCA-gap-EGFP were previously described (Okada et al., 2006). pEGFP-mPtchl, mGli1 and pcDNA3-mGas1 were kindly provided by C. C. Hui and V. Wallace (Lee, C. S., Buttitta, L. & Fan, C. M., 2001. Evidence that the WNT-inducible growth arrest-specific gene 1 encodes an antagonist of sonic hedgehog signaling in the somite. Proceedings of the National Academy of Sciences of the United States of America, 98(20), pp. 11347-11352. Chang, H. et al., 2010. Activation of Erk by sonic hedgehog independent of canonical hedgehog signalling. The international journal of biochemistry & cell biology, 42(9), pp. 1462-1471). pEGFP-Smo was kindly provided by P. Beachy.

Histology and Immunohistochemistry.

β-gal activity detection and immunochemistry on sections were performed according to protocols described previously (Charron et al., 2003; Fabre et al., 2010; Okada et al., 2006). Antibody dilutions: rabbit anti-mouse Lim1 (1:500, T. Jessell lab), rabbit anti-mouse Pax6 (1:100, Chemicon), rabbit antimouse Calbindin (1:200, Chemicon), goat anti-mouse TAG1 (1:400), rabbit anti-GFP (1:1000, Invitrogen), goat anti-mouse Boc (1:100, R&D), goat anti-mouse Cdon (1:250, R&D), goat anti-mouse Gas1 (1:200, R&D). Prior to performing immunostainings with anti-Boc antibody, sections were subjected to antigen retrieval for 1 hr at 98° C. in a sodium citrate buffer (10 mM sodium citrate, 0.05% Tween 20 [pH 6.0]), cooled at room temperature for 20 min and washed extensively with PBS.

Isolation of CGNPs and In Vitro Proliferation Assays.

CGNPs were isolated from either E18.5 or P4 mouse or rat cerebella using a modified protocol previously described (Wechsler-Reya and Scott, 1999). Briefly, isolated cerebella were cut in small pieces and treated with 0.25% trypsin and DNase I. Following trituration, single cell suspensions were centrifuged through a 30% to 65% Percoll step gradient. Cells harvested at the 30% interphase were resuspended in Neurobasal supplemented with B27, 0.5 mM L-Glutamine and Pen/Strep and plated in 96-well plates precoated with 100 μg/ml poly-D-Lysine. For CGNP proliferation assays with IGF-1, cells were resuspended in Neurobasal supplemented with 0.06% D-glucose, 100 μg/ml apo-transferrin, 16 μg/ml putrescine, 30 nM sodium selenite, 20 ng/ml progesterone, and 1 mg/ml BSA. For ³H-thymidine incorporation assays, cells were seeded at 2 or 4×10⁵ cells/well in a 96-well plate in triplicate and treated with ShhN C24II for 48 hr. CGNPs were pulsed with 1 μCi/ml ³H-thymidine (PerkinElmer) for the last 12 hr. Incorporation was measured using the Filtermate™ harvester (PerkinElmer) and TopCount™ NXT beta counter (Packard). Alternatively, CGNPs were seeded at 2.5×10⁴ cells/well of a 96-well/plate and cultured as described above. Following 48 hr in culture, cells were fixed with 4% PFA, blocked with 10% PHT, and immunostained with mouse anti-Ki67 antibody (1:100, Becton Dickinson).

GFP Reporter Mouse Line.

The GFP reporter mouse line mTmG (Gt(ROSA)26Sor^{tm4(ACTB-tdTomato,-EGFP)Luo}/J) was generated by L. Luo (Muzumdar et al., 2007) and was obtained from the Jackson Laboratory. This line expresses membrane-targeted GFP (mG) after Cre-mediated excision.

In Vivo Proliferation Assays and TUNEL Labeling.

P3 mice littermates were injected intraperitoneally with 100 mg/kg BrdU (Sigma) 30 min prior to euthanasia. Brains were dissected and fixed with 4% PFA. Sections were immunostained with rat anti-BrdU antibodies (1:200, Abcam). CGNPs in the EGL were also immunostained for phospho-histone H3 (pH3) (rabbit anti-pH3, 1:500, Upstate). TUNEL assays were performed using the Apoptag-Red™ in situ apoptosis detection kit (Chemicon).

In Situ Hybridization.

Gli1 cDNA plasmid used for generating the riboprobe was a generous gift from C. C. Hui. DIG-labeled Gli1 RNA probe was synthesized by in vitro transcription with T3 RNA polymerase using a DIG RNA labeling kit (Roche) according to the manufacturer's instructions. In situ mRNA detection was performed as previously described (Kao et al., 2009).

Cell Culture.

C3H 10T½-gli-luc cells were kindly provided by J. Filmus. HEK 293, NIH 3T3, COS7, C3H 10T½ and C3H 10T½-gli-luc cells were maintained in DMEM, 10% FBS and penicillin/streptopmycin (Gibco).

Immunoprecipitations.

COS7 cells were plated at 1×10⁶ cells/100 mm-dish and transfected the next day with expression plasmids for the indicated proteins. Immunoprecipitations and immunoblots were performed as previously described (Okada et al., 2006).

Site Directed Mutagenesis and ShhN Production.

Mutagenesis was performed using the QuickChange™ Site-Directed Mutagenesis Kit (Stratagene). COS7 cells were plated at 1×10⁶ cells/100-mm dish and transfected with Shh expression plasmids using Lipofectamine™ 2000 (Invitrogen). Culture medium was replaced with Opti-MEM™ I with penicillin/streptopmycin (Gibco) 24h post-transfection. Supernatant was harvested 48h later and buffered with 40 mM HEPES. 5E1 hybridoma cells were obtained from the Developmental Studies Hybridoma Bank, University of Iowa, and were maintained in IMDM, 10% FBS and 50 μg/ml gentamicin (Gibco). Monoclonal anti-Shh 5E1 antibody was produced by plating 5E1 hybridoma cells at 2.5×10⁵ cells/100-mm dish and harvesting culture media 9 days later. Concentrated 5E1 antibody was cross-linked to immobilized Protein G resin (Pierce) according to manufacturer's instructions. For ShhN purification, 5E1 resin was incubated with ShhN conditioned media (WT or mutant), washed with 10 mM phosphate pH 6.8 and eluted with 100 mM glycine pH 2.6 in a stepwise fashion. Fractions were collected in LoBind™ tubes (Eppendorf), buffered and analyzed by SDS-PAGE and Coomassie blue staining.

Receptor Binding Assays.

Shh-receptor binding assays using pEGFP-mPtch1, pEGFP-mBoc, pEGFP-mCdon and pcDNA3-mGas1 were performed according to previously described protocols (Okada et al., 2006). For Boc/Ptch1 binding assays, COS7 cells expressing either mPtch1, Disp1 or Smo were incubated with Boc ecto-Fc (or deletion mutants) diluted in binding buffer A (BBA; DMEM, 10% FBS, 0.1% NaN2, 50 mM HEPES). Following incubation, cells were washed extensively with BBA, followed by PBS, fixed with 2% PFA/PBS, and blocked with 10% heat inactivated goat serum/PBS (HIGS). Cells were incubated with goat anti-human IgG-HRP in 10% HIGS (1:5000, Chemicon) for 1 h at RT, washed extensively with PBS and incubated with Sigma FAST OPD reagent.

Luciferase Reporter Assay.

C3H 10T½-gli-luc cells were plated at 2×10⁴ cells/well in a 96-well plate, treated with purified ShhN (WT or mutant) 48h later. The next day, cells were harvested and processed as previously described (Morin et al., 2000).

C3H 10T½ Differentiation Assay.

C3H 10T½ differentiation assays were performed as previously described (Traiffort et al., 2004) with the following modifications. Cells were treated with purified ShhN (WT or mutant) for 96 h and harvested in 10 mM Tris pH 8.0, 1% Triton™ X-100 buffer. AP activity was detected with pNPP substrate (Sigma).

Cell Surface Biotinylation.

COS7 cells transfected as indicated were labeled with biotin or indicated antibodies as previously described (Zheng et al., 2010). Antibody dilutions used: goat anti-mouse Boc (1:100; R&D); goat anti-mouse Gas1 (1:250; R&D).

Cell Surface Immunostaining.

COS7 or NIH 3T3 cells were transfected with Boc, Gas1, and Ptch1-GFP expression vectors as indicated. Twenty-four hours post-transfection, cells were fixed with 4% PFA and immunolabeled without permeabilization with either anti-Boc or anti-Gas1 antibodies directed against the extracellular epitopes of Boc and Gas1. Ptch1 localization was visualized via GFP.

Statistical Methods.

Unless otherwise noted, all data are expressed as mean±SEM.

The statistical tests used to measure differences are indicated in the appropriate legends. Statistical significances are indicated as follows: *p<0.05; **p<0.01; ***p<0.001; zp <0.0001; n.s., not significant.

### Example 2

### Boc, but not Cdon, is Expressed in Proliferating CGNPs of the Cerebellum

To investigate the receptor requirements for the proliferative effect of Shh in CGNPs, we first analyzed Boc and Cdon expression in the developing cerebellum. CGNPs arise from the rhombic lip (RL) between embryonic day (E) 13.5-14.5 and migrate anteriorly over the cerebellar anlage, forming the highly proliferative external germinal layer (EGL) (Ben-Arie et al., 1997; Hatten and Heintz, 1995; Zervas et al., 2005). Starting at E17.5 and continuing during early postnatal development, Purkinje cells (PCs) lining the EGL stimulate CGNP proliferation by secreting Shh (Dahmane and Ruiz i Altaba, 1999; Kenney and Rowitch, 2000; Wallace, 1999; Wechsler-Reya and Scott, 1999). Following a proliferative burst, CGNPs stop dividing, differentiate into granular neurons, migrate inwards past the PC layer and populate the internal granular layer (IGL).

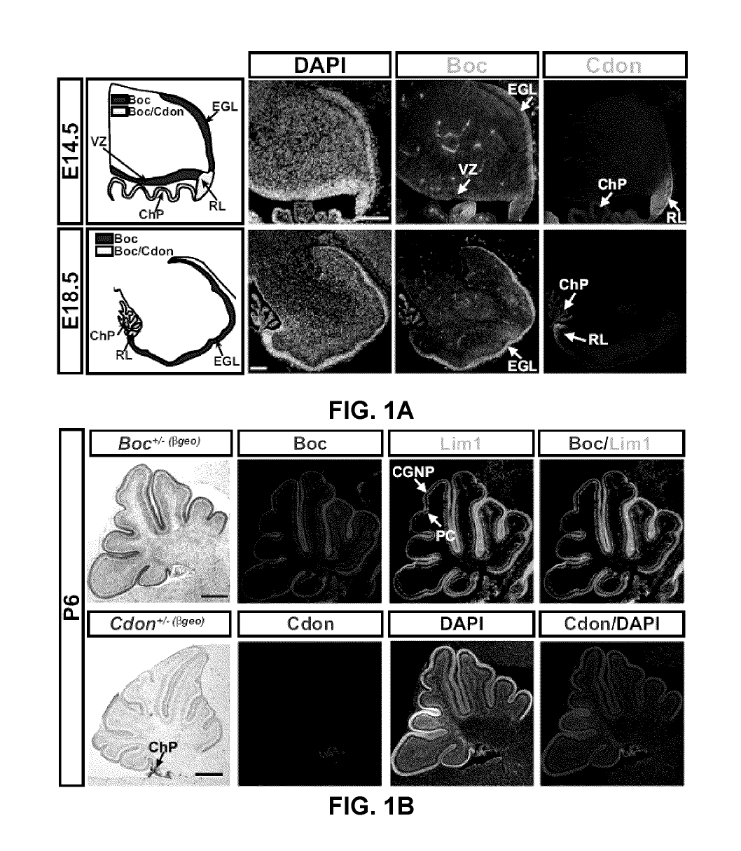

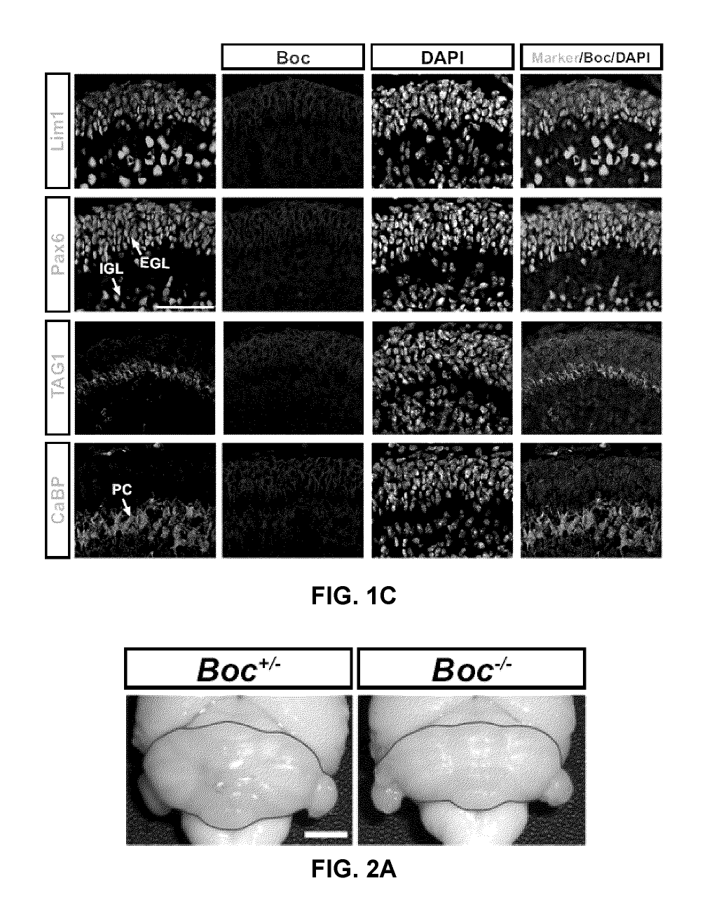

We first examined Boc and Cdon expression in the cerebellar anlage of E14.5 mouse embryos. Immunostainings of sagittal sections showed that while Boc was expressed in the presumptive EGL, RL and the ventricular zone of the roof of the 4^{th} ventricle, Cdon expression was restricted to the RL (FIG. 1A). At E18.5, a stage at which CGNPs proliferate in response to Shh, we detected Boc expression in the EGL and, albeit at lower level, in the PC layer of the developing cerebellum. In contrast, Cdon expression was limited to the tip of the RL.

Analysis of post-natal day (P) 6 Boc^{+/-} and Cdon^{+/-} gene-targeted mice encoding a β-galactosidase (β-Gal)-neomycin reporter gene fusion (β-geo) (Okada et al., 2006) revealed strong β-Gal activity in Boc^{+/-} cerebellum, but was limited to the choroid plexus of Cdon^{+/-} cerebellum. Immunostainings confirmed this expression pattern and revealed that Boc localized to cells expressing Lim1, a marker for CGNPs and PCs (FIGS. 1B, C). Interestingly, while highest levels of Boc were detected in the outer proliferative region of the EGL (Lim1⁺, Pax6⁺ and TAG1⁻ cells), lower levels were observed in differentiated migratory granule cells (TAG1⁺ cells) and in PCs (Calbindin⁺ cells). These results show that Boc, but not Cdon, is highly expressed in proliferating CGNPs of the cerebellum.

### Example 3

### Boc is Involved in Shh-Mediated CGNP Proliferation

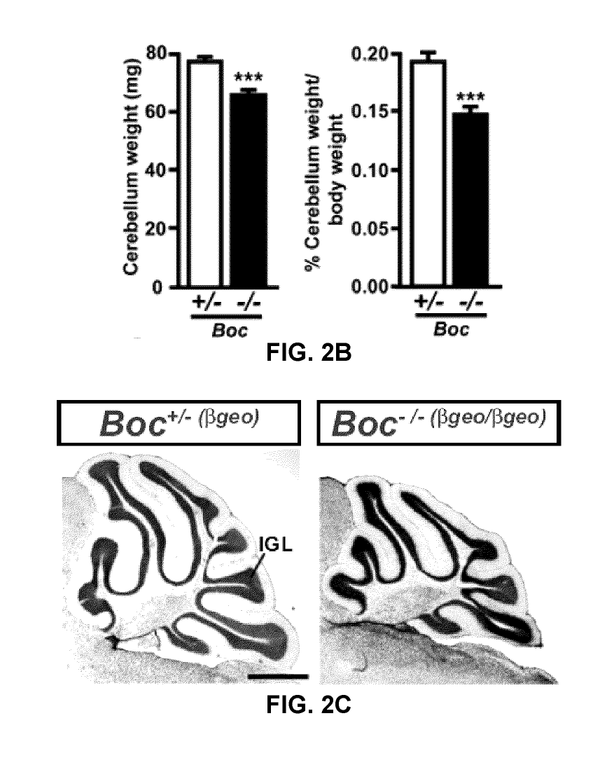

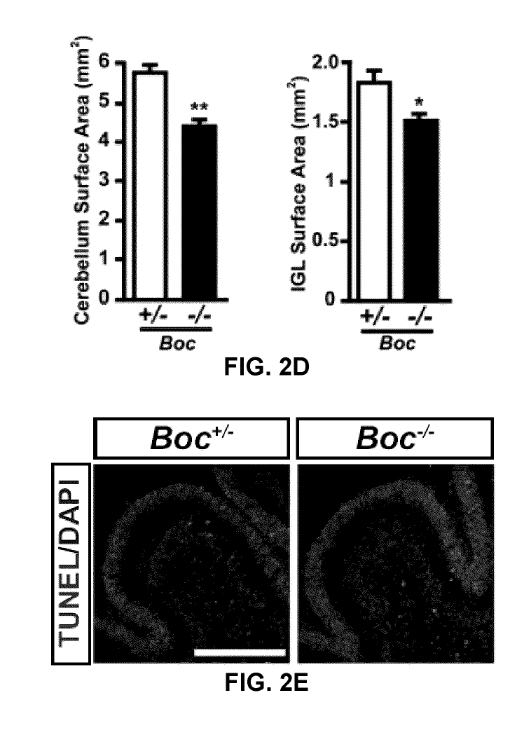

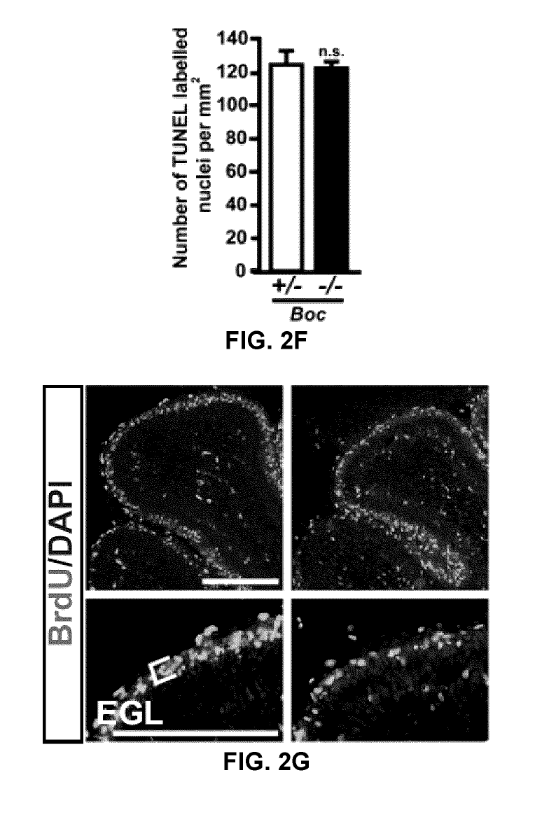

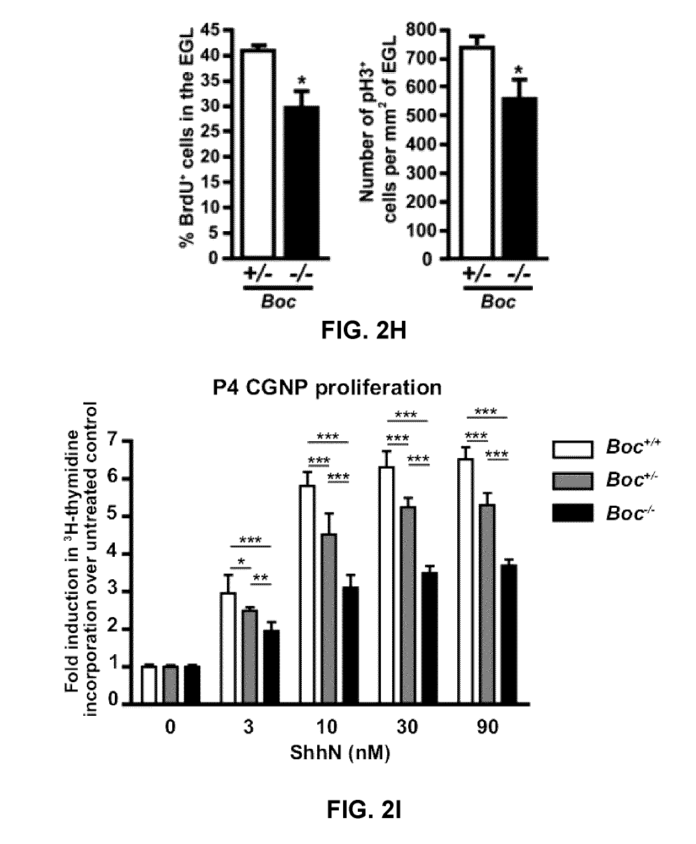

To investigate the role of Boc in cerebellum development, we examined the gross morphology of Boc^{-/-} cerebella. While Boc^{+/-} mice are viable and cannot be distinguished from their littermates, their cerebellum is smaller than Boc^{+/-} or WT animals (FIG. 2A). Boc^{-/-} cerebella were 14.3±0.05% (p<0.001) lighter than that of Boc^{+/-} cerebella (FIG. 2B). When the mass of the cerebellum was normalized to the body weight (p<0.001), the relative cerebellar mass was still reduced, indicating that this difference is not due to an overall decrease in total body weight (FIG. 2B). The cerebellum and IGL surface areas measured from sagittal sections of Boc^{-/-} adult mice were also reduced when compared to Boc^{+/-} animals (FIGS. 2C, D; p<0.001 and 0.05, respectively). Although the IGL surface area is diminished in adult mice, migration of granule neurons and cerebellum foliation did not appear to be affected in Boc^{-/-} mice.

The decrease in cerebellum size in the absence of Boc could be due, at least in part, to reduced cell proliferation and/or enhanced cell death. TUNEL staining showed no significant difference in the number of apoptotic cells between Boc^{-/-} and Boc^{+/-} cerebella (FIGS. 2E, F). In contrast, measurement of BrdU incorporation in the EGL of Boc^{-/-} and Boc^{+/-} mice showed that 40±1% of Boc^{+/-} CGNPs were actively dividing, compared to only 30±3% of Boc^{-/-} CGNPs (p<0.05) (FIGS. 2G, H). Phospho-histone H3 (pH3) staining also showed a significant reduction in the number of mitotic pH3-labeled cells per mm² of EGL in Boc^{-/-} mice compared to Boc^{+/-} mice (p<0.05) (FIG. 2H). Together, these in vivo data indicate that Boc plays a role in CGNP proliferation.

Since Boc modulates Shh signaling (Okada et al., 2006; Tenzen et al., 2006; Zhang et al., 2006), we next tested whether Boc mediates Shh-induced CGNP proliferation. We cultured CGNPs purified from Boc^{-/-}, Boc^{+/-} and Boc^{+/+} mice in the presence of varying concentrations of recombinant Shh (ShhN) (FIG. 2I). While Shh treatment induced the proliferation of WT CGNPs over 6 fold compared to unstimulated CGNPs, Shh stimulation increased Boc^{-/-} CGNP proliferation only about 3 fold. Significant differences in the proliferation of Boc^{+/+}, Boc^{+/-} and Boc^{-/-} CGNPs was observed at all concentrations of ShhN used (FIG. 2I), indicating that Boc promotes proliferation of CGNPs in a gene copy-number dependent manner. Together with our in vivo data, these results indicate that Boc^{-/-} mice have a smaller cerebellum due to a decrease in Shh-dependent CGNP proliferation and that Boc acts cell-autonomously in CGNPs to regulate their proliferation.

### Example 4

### GAS1 is Also Involved in Shh-Mediated CGNP Proliferation

Whilst inactivation of Boc in CGNPs, which do not express Cdon, lead to a partial decrease in their proliferation, it did not abolish their response to Shh. Moreover, CGNP proliferation is not further decreased when Cdon is inactivated in Boc^{-/-} mice (FIG. 8). These results are not consistent with a model where Boc and Cdon act like their Drosophila orthologues Ihog and Boi and are absolutely required for Hh signaling in vertebrates (Camp et al., 2010; Zheng et al., 2010). This raises the possibility that, unlike Drosophila, additional or different Shh binding molecules (other than PTCH1, BOC and Cdon) are required for vertebrate cells to respond to Shh.

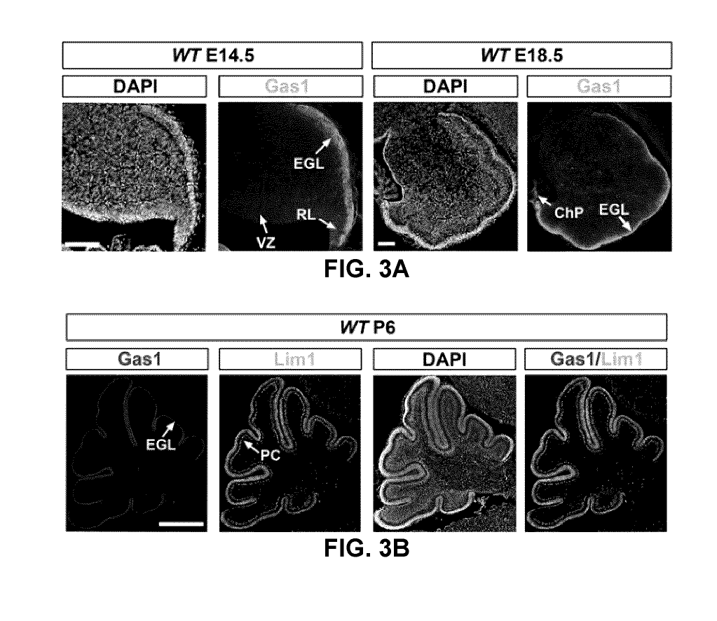

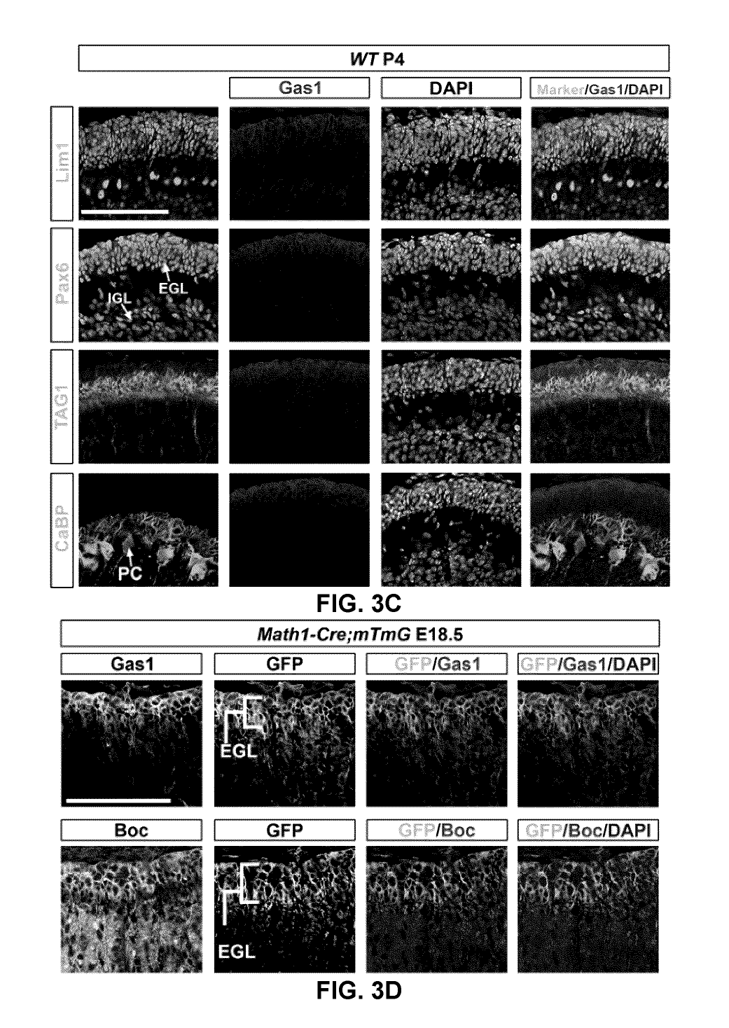

Given that GAS1 binds Shh and modulates Shh signaling (Allen et al., 2007; Martinelli and Fan, 2007a, b; Seppala et al., 2007), we hypothesized that GAS1 may be this additional receptor. We first characterized the expression pattern of Gas1 in the developing cerebellum. Immunofluorescence stainings showed that Gas1 is restricted to the presumptive EGL of the cerebellar primordium at E14.5 and continues to be expressed in the EGL at E18.5 (FIG. 3A). At P6, like Boc, Gas1 localizes to Lim1⁺ cells in the EGL (FIG. 3B). Gas1 staining is most intense in the outer proliferative layer of the EGL (Lim1⁺, Pax6⁺, TAG1⁻ cells) and was not detected in TAG1⁺ migratory granule neurons and in Calbindin⁺ PCs (FIG. 3C).

To determine whether Boc and Gas1 are co-expressed in CGNPs, we performed immunostainings on consecutive sections of cerebellum from Math1-Cre; mTmG E18.5 mice, where the CGNPs express GFP following Cre-mediated recombination. We used this strategy instead of double immunostainings as both anti-Boc and anti-Gas1 antibodies are produced in the same species. We found that both Boc and Gas1 co-localize with GFP+ cells, indicating that Gas1 and Boc are co-expressed in the same CGNPs (FIG. 3D).

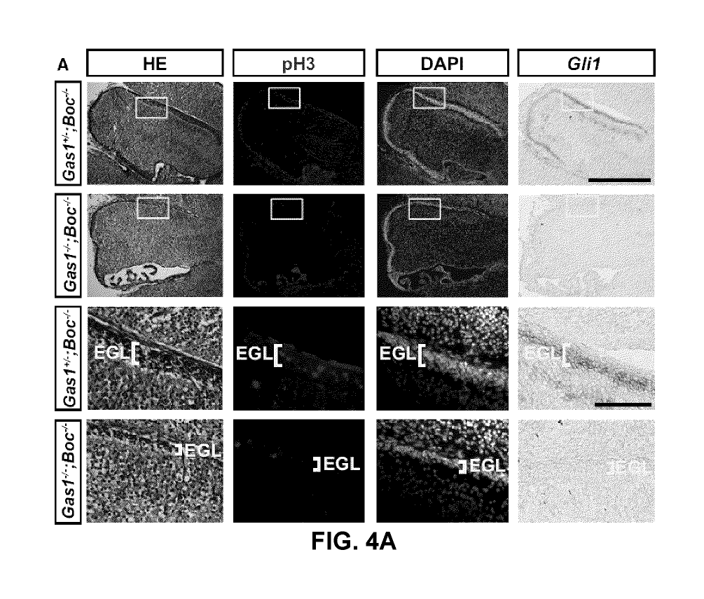

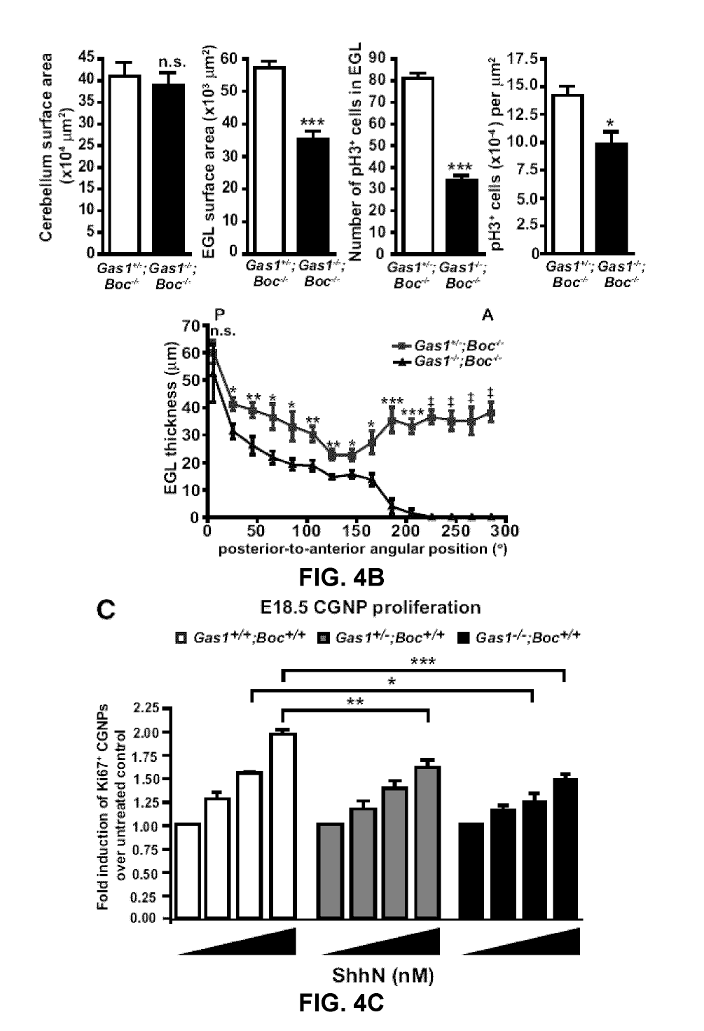

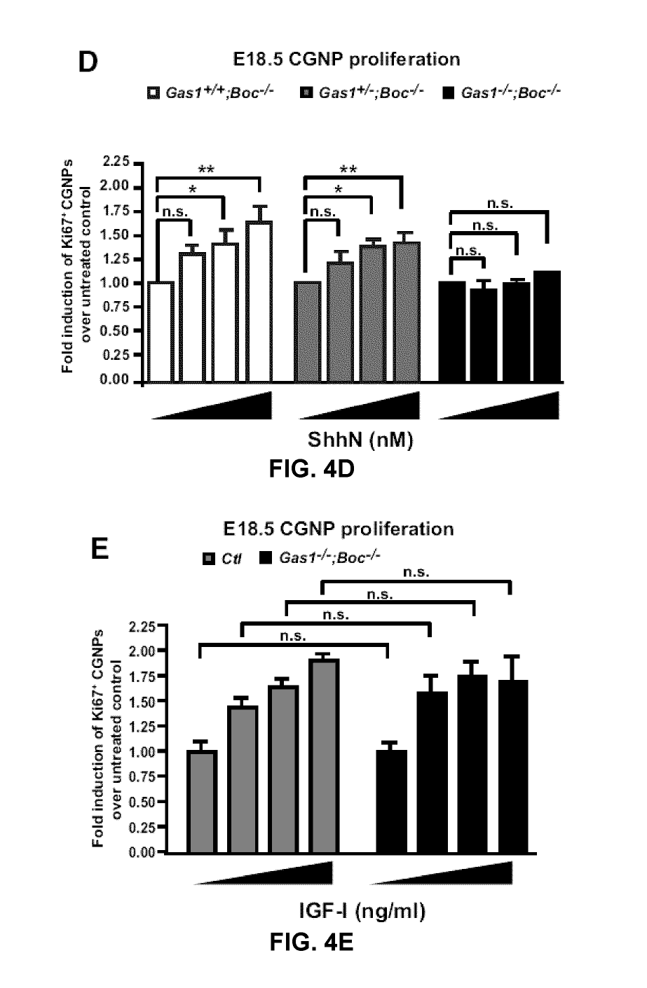

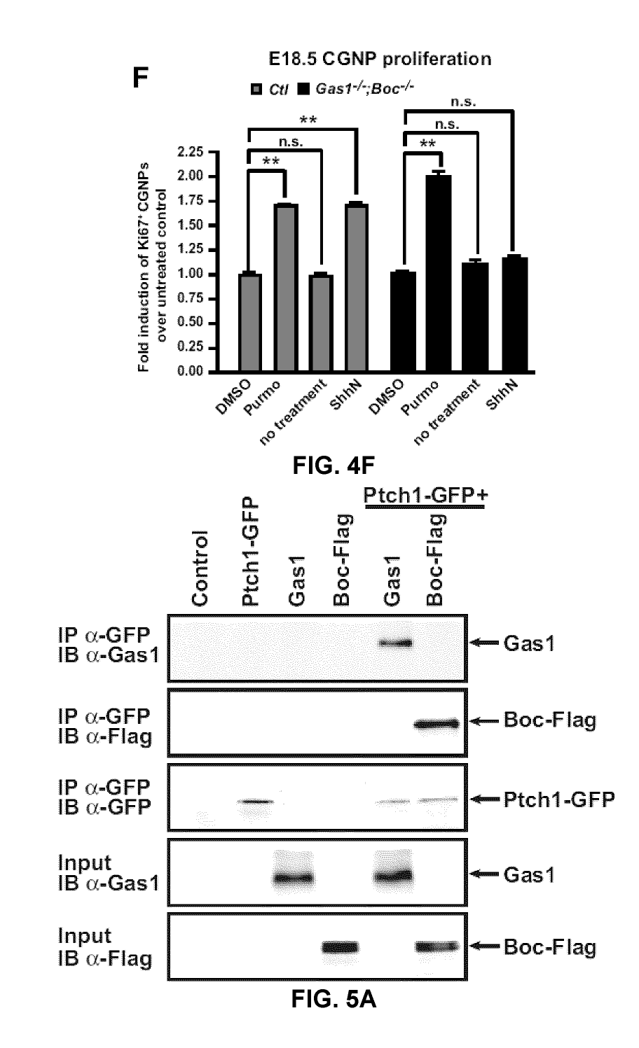

Although the gross morphology of Gas1^{-/-} cerebella appears normal, they are smaller in size compared to control cerebellum and have decreased proliferation in the outer EGL (Liu et al., 2001). While this phenotype is reminiscent of that of Boc^{-/-} cerebella, no direct link has been made between the phenotype and the ability of Gas1^{-/-} CGNPs to respond to Shh. To directly test this, we performed proliferation assays on purified CGNPs from Gas1^{-/-} mice and control littermates. Our results show that Gas1 is essential for normal CGNP proliferation in response to Shh (FIG. 4C). Interestingly, the mutation of Gas1, similarly to the mutation of Boc, is not sufficient to abrogate the response of CGNPs to Shh.

### Example 5

### Shh-Dependent Proliferation is Completely Lost in Gas1^{-/-};Boc^{-/-} CGNPs

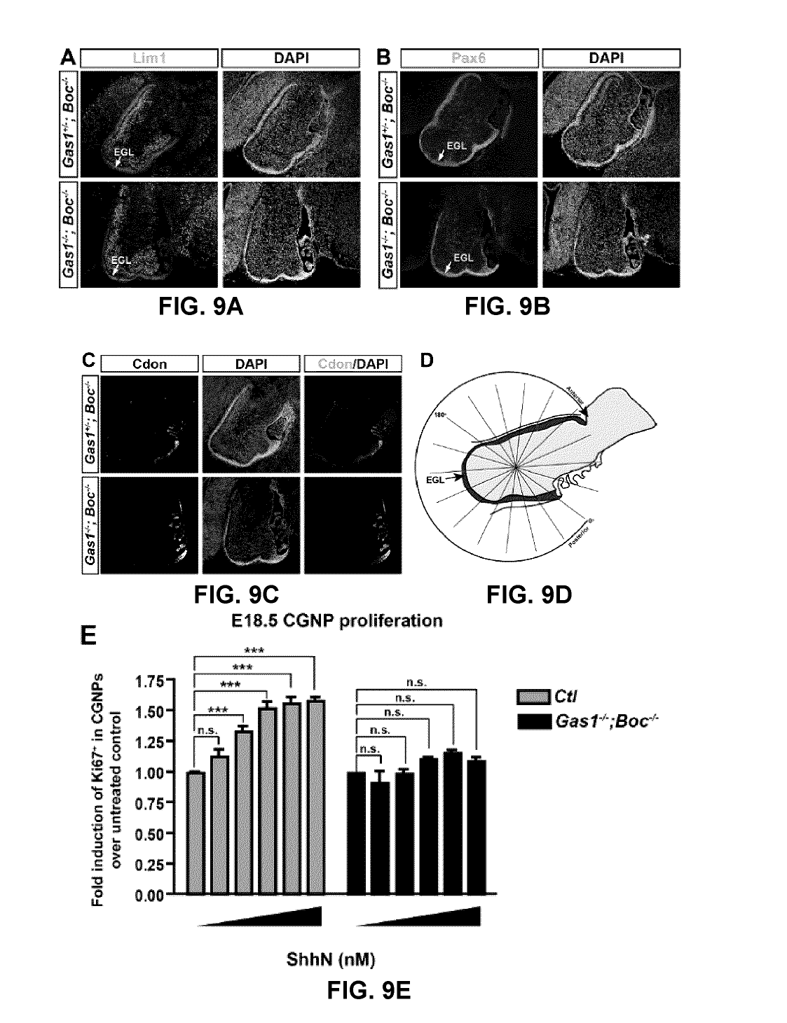

To determine whether Boc and Gas1 might have partially redundant functions in Shh-dependent CGNP proliferation, we examined the cerebellum of E18.5 Gas1^{-/-};Boc^{-/-} embryos, since these animals die at birth. Hematoxylin-eosin staining of Gas1^{-/-};Boc^{-/-} cerebella revealed a significant loss of the EGL compared to controls (FIG. 4A). Although Gas1^{+/-};Boc^{-/-} and Gas1^{-/-};Boc^{-/-} cerebella showed no significant difference in the cross-sectional area of the whole cerebellum, the overall area of Gas1^{-/-};Boc^{-/-} EGL was reduced by about 30% compared to controls (p<0.001) (FIG. 4B). Quantitation of the EGL along the postero-anterior axis showed that the difference in EGL thickness is greatest towards the anterior pole of the cerebellum (FIGS. 4B and 9D). Marker analysis showed that Lim1 and Pax6 were properly expressed in the EGL of Gas1^{-/-};Boc^{-/-} embryos compared to controls (FIGS. 9A,B), thus, CGNPs are specified and localize normally. Furthermore, Cdon expression was not changed in the absence of Gas1 and Boc (FIG. 9C). However, the proliferation of Gas1^{-/-};Boc^{-/-} CGNPs was severely decreased compared to Gas1^{+/-};Boc^{-/-} CGNPs (p<0.001) (FIG. 4B). Moreover, the number of pH3⁺ cells per μm² of EGL surface area was lower in Gas1^{-/-};Boc^{-/-} than Gas1^{+/-};Boc^{-/-} animals (p<0.05) (FIG. 4B), demonstrating that the decrease in pH3⁺ cells in the EGL is not simply due to a total decrease in EGL area. These results indicate that Gas1 and Boc account for a large part of CGNP proliferation at this stage in vivo.

In addition to Shh, Insulin Growth Factor (IGF) and Notch signaling also promote CGNP proliferation (Corcoran et al., 2008; Fernandez et al., 2010; Solecki et al., 2001). Residual CGNP proliferation is observed in other mutant cerebella that lack Shh signaling (Corrales et al., 2004), thus, the proliferation observed in the EGL of Gas1^{-/-};Boc^{-/-} cerebellum is probably independent of Shh signaling. To test whether Gas1^{-/-};Boc^{-/-} cells have completely lost Shh responsiveness, we cultured CGNPs purified from E18.5 Gas1^{+/+};Boc^{-/-} and Gas1^{-/-};Boc^{-/-} cerebella with various ShhN concentrations. We found that while Gas1^{+/+};Boc^{-/-} CGNPs proliferate in vitro in response to Shh, Gas1^{-/-};Boc^{-/-} CGNPs show no enhanced proliferation in response to Shh (FIG. 4D). Importantly, the proliferative response of Gas1^{-/-};Boc^{-/-} CGNPs to IGF-I, another factor able to stimulate CGNP proliferation, remained similar to that of control cells (FIG. 4E). Furthermore, treatment with purmorphamine, a Smo agonist, induced the proliferation of Gast^{-/-};Boc^{-/-} CGNPs (p<0.01) (FIG. 4F), indicating that Boc and Gas1 function upstream of Smo. Together, our data indicates that the presence of either Gas1 or Boc is absolutely required for Shh to promote CGNP proliferation. Given that Shh signaling in the cerebellum begins only at E17.5 and that Shh signaling plays an even more important role in CGNP proliferation after birth than at E18.5 (Corrales et al., 2004; Flora et al., 2009; Lewis et al., 2004), we anticipate that the EGL of Gas1^{-/-};Boc^{-/-} mice would be much more severely reduced post-natally.

To test whether the lack of a proliferative response of Gas1^{-/-;Boc}^{-/-} CGNPs to Shh in vitro is consistent with loss of Shh signaling in vivo, we examined the expression of Gli1, a Shh transcriptional target (Corrales et al., 2004), by RNA in situ hybridization. While control cerebella had intense Gli1 signal in the EGL, Gli1 expression was not detected in Gas1^{-/-};Boc^{-/-} cerebella (FIG. 4A), confirming the inactivation of Shh signaling in Gas1^{-/-};Boc^{-/-} cerebella.

### Example 6

### BOC and GAS1 Interact with PTCH1 and Form Distinct Receptor Complexes

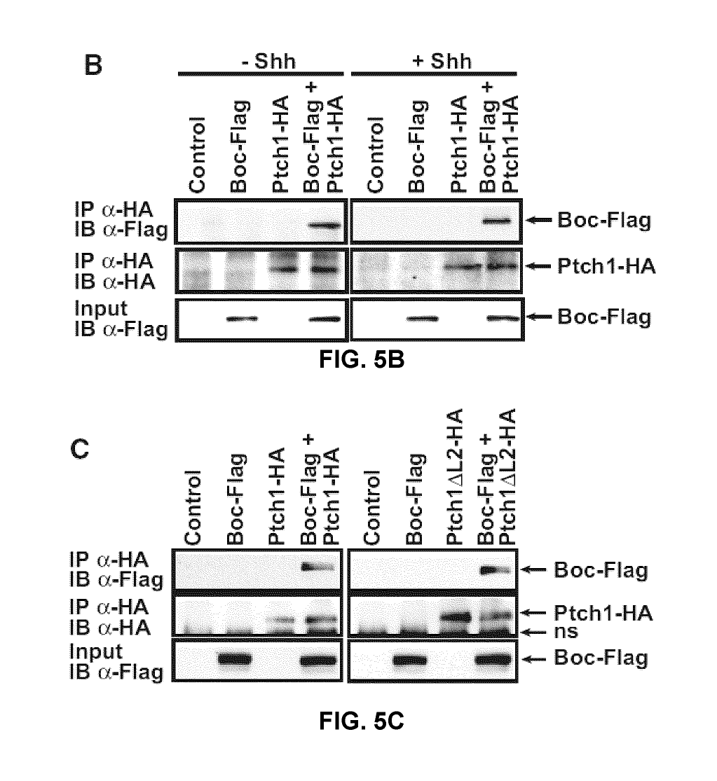

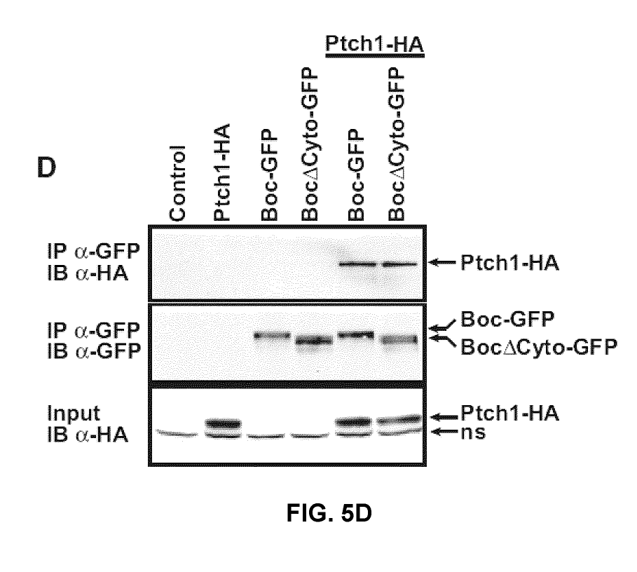

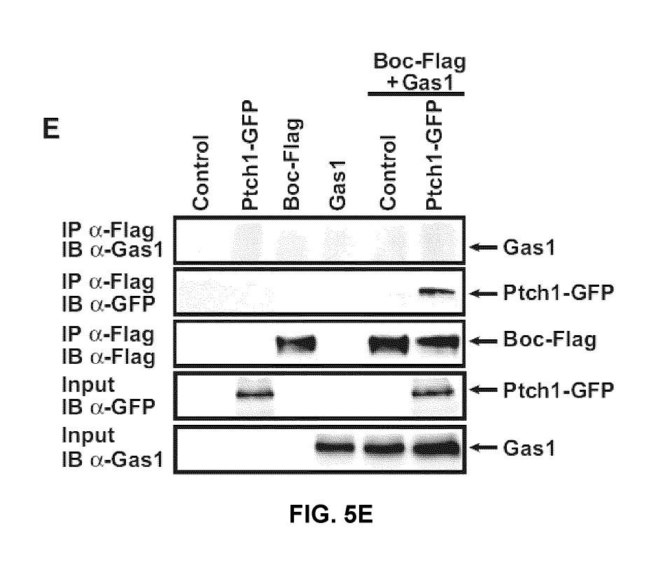

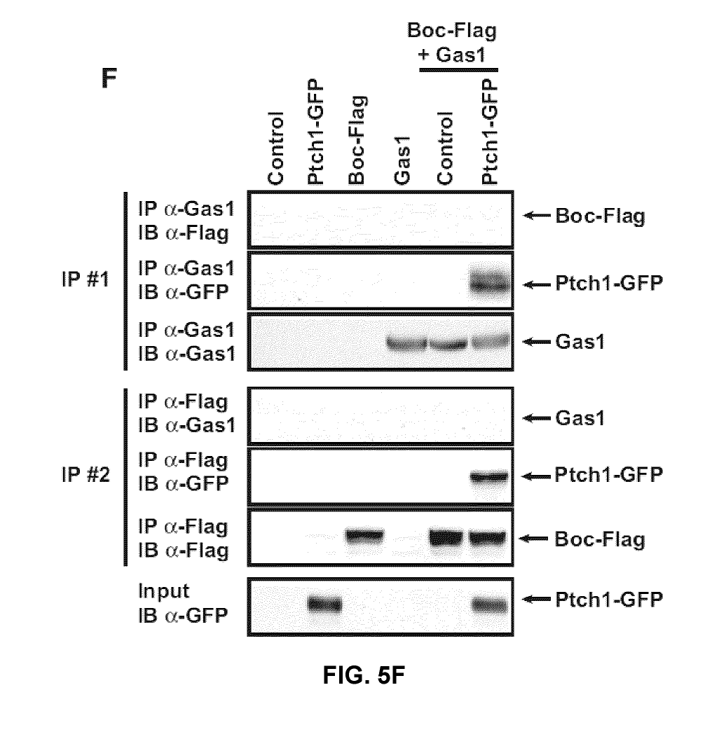

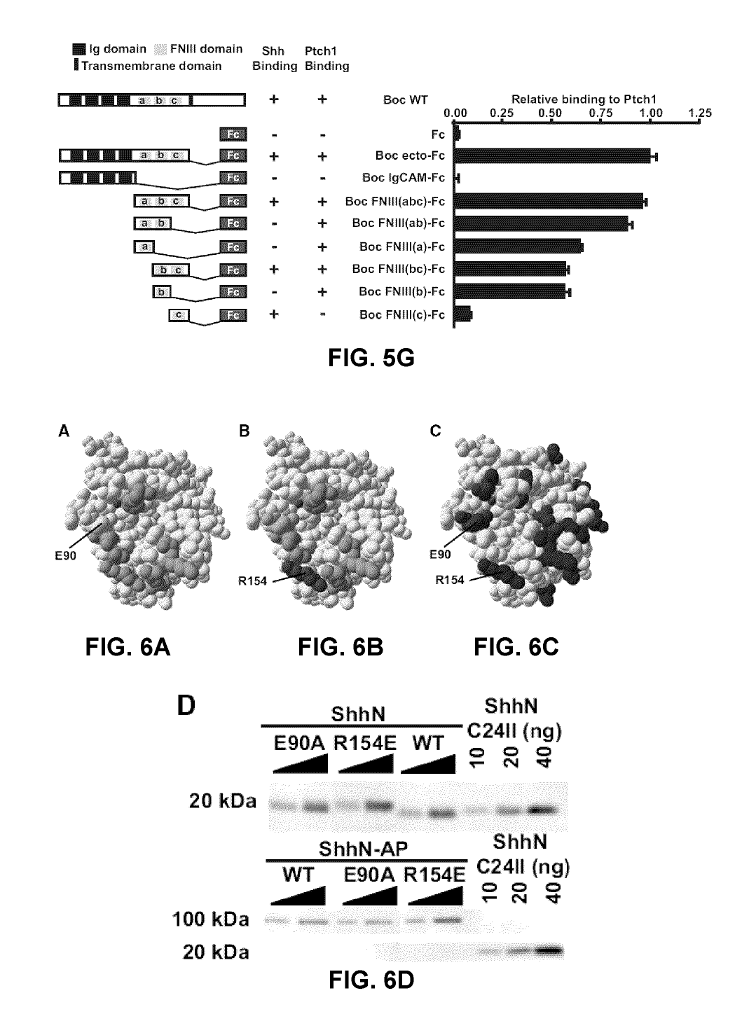

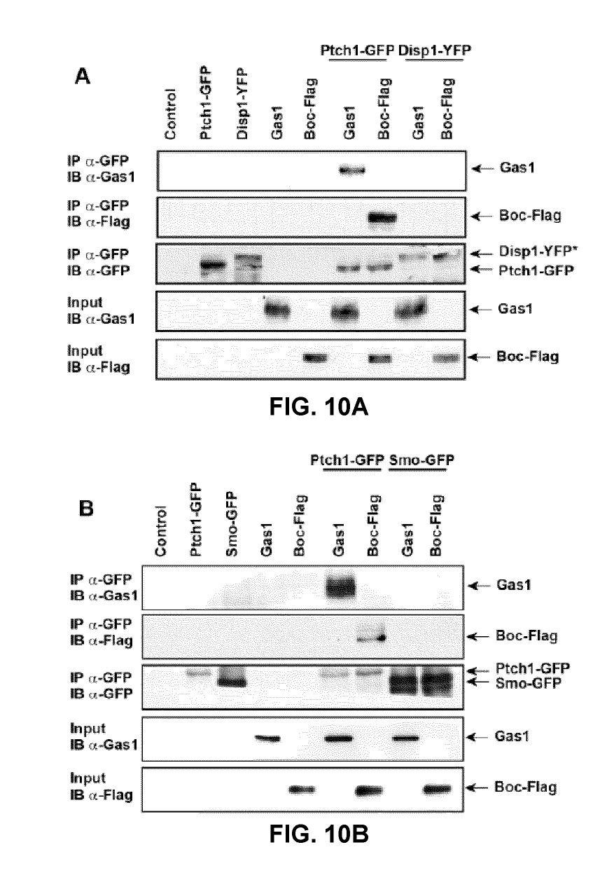

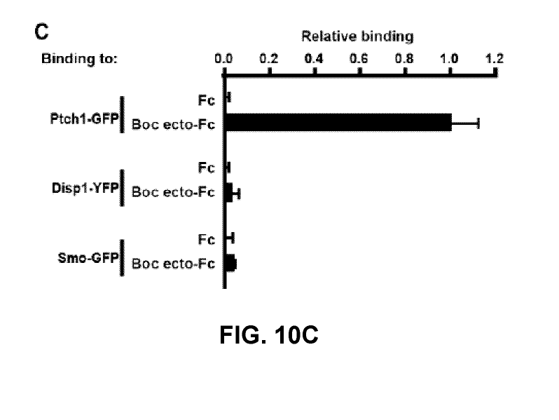

We next investigated the molecular mechanism by which BOC and GAS1 act and, more specifically, whether they associate with PTCH1 to constitute the Shh receptor complex. We found that BOC and GAS1 can each co-immunoprecipitate with PTCH1, indicating that BOC and GAS1 can physically interact with PTCH1 (FIG. 5A). Importantly, these interactions are specific to PTCH1, as both Dispatched-1 (Disp1) and Smo, two multi-span transmembrane proteins also involved in Shh signaling, failed to interact with either BOC or GAS1 (FIG. 10). Furthermore, the addition of Shh did not modify the ability of PTCH1 to interact with BOC, suggesting that their interaction is constitutive (FIG. 5B).

Mapping studies showed that the second large extracellular loop of PTCH1 (L2), which is necessary for binding to Shh (Marigo et al., 1996), was not required for the interaction with BOC. PTCH1ΔL2-HA, a PTCH1 construct where L2 is deleted, interacted with BOC to an extent similar to full length PTCH1-HA (FIG. 5C). This is consistent with the binding of Shh to PTCH1 not being necessary for PTCH1 to interact with BOC. We next mapped the domain(s) of BOC mediating its interaction with PTCH1. BOCΔCyto-GFP, a mutant lacking the cytoplasmic domain of BOC, interacted with PTCH1 as strongly as full-length BOC-GFP (FIG. 5D), indicating that the cytoplasmic domain is not involved in its association with PTCH1.

To further characterize the region of BOC that interacts with PTCH1, we performed binding assays with various derivatives of BOC-Fc fusion proteins encompassing the BOC extracellular domain and cells expressing PTCH1-GFP. Deletion analysis of the BOC extracellular domain revealed that removal of the FNIIIc domain (mutant BOC FNIII(ab)), shown to be involved in Shh binding (Okada et al., 2006), only marginally affected PTCH1 binding, while truncation of both the FNIIIa and FNIIIb domains (mutant FNIII(c)) abolished it almost entirely (FIG. 5G). BOC-Fc constructs containing either the FNIIIa or FNIIIb domains alone bound to PTCH1 at levels that were about 60% of that of BOC ecto-Fc. Together our data indicate that the BOC FNIIIa and FNIIIb domains are required and sufficient to mediate its interaction with PTCH1. In addition, the BOC FNIIIc domain, which is involved in Shh binding, is not involved the BOC-PTCH1 interaction, further supporting a Shh-independent interaction between BOC and PTCH1.

We next tested whether BOC interacts with GAS1 and did not detect an interaction between BOC and GAS1 either in the absence or presence of PTCH1 (FIG. 5E, top panel, lanes 5-6), despite detecting a strong interaction between BOC and PTCH1 (FIG. 5E, middle panel, lane 6). These experiments suggest that BOC/PTCH1 complexes do not contain detectable amounts of GAS1 and that BOC, PTCH1 and GAS1 are unlikely to form a tripartite complex.

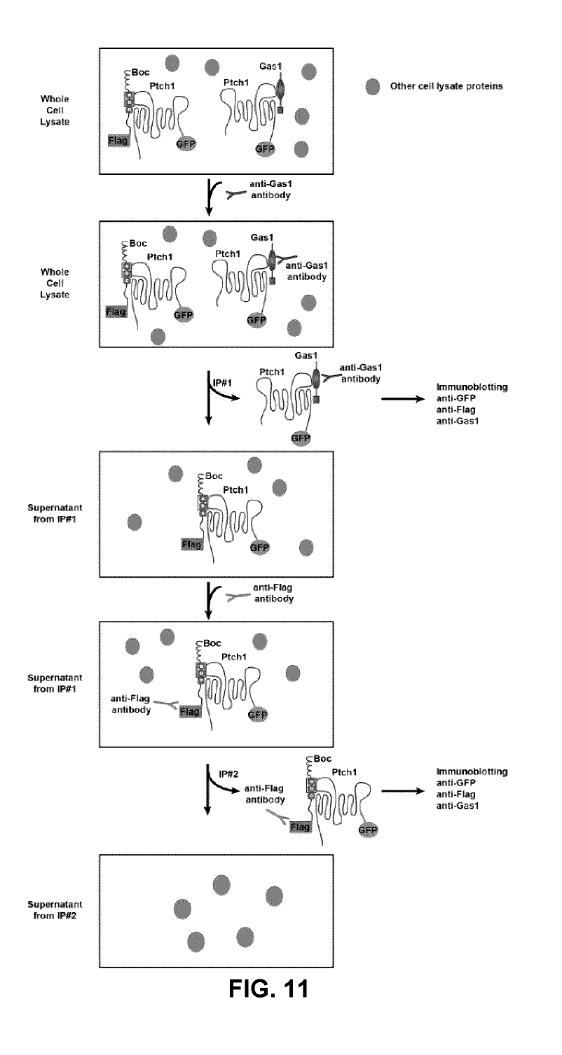

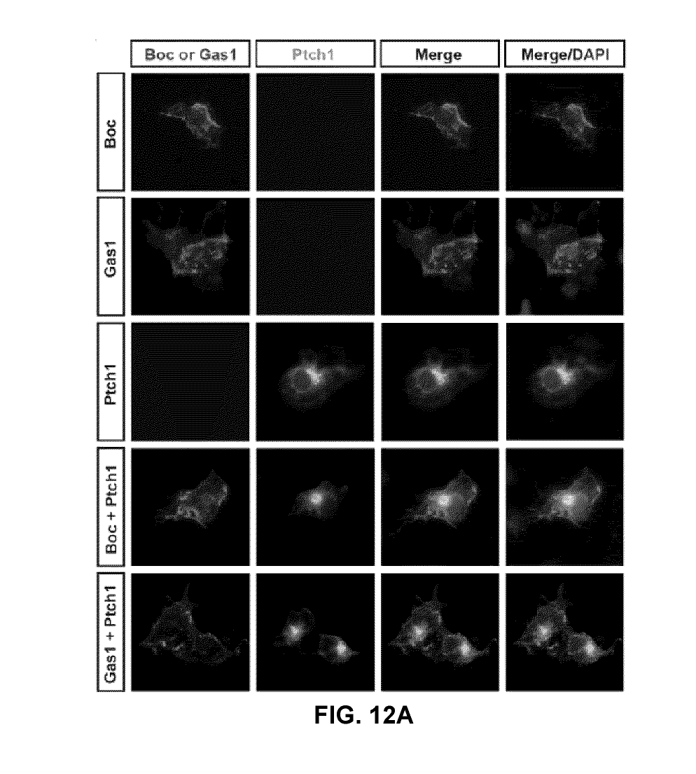

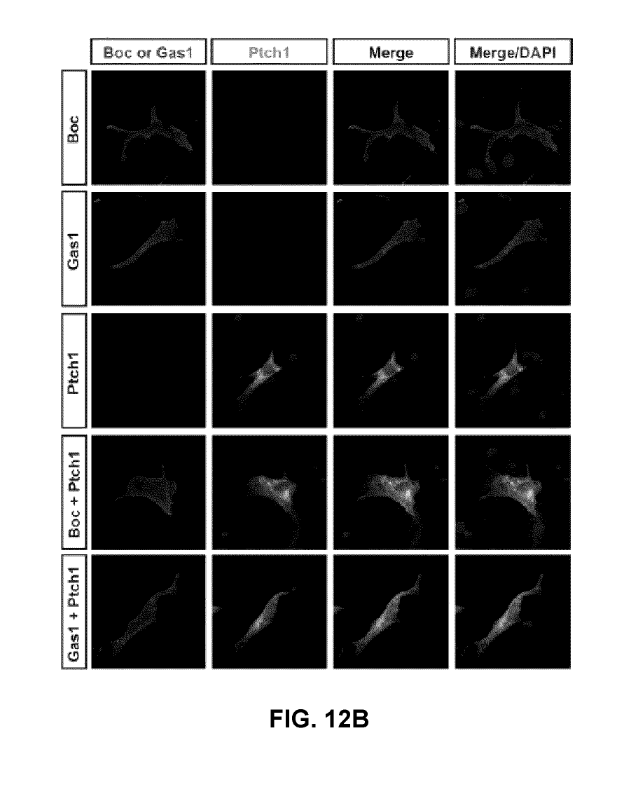

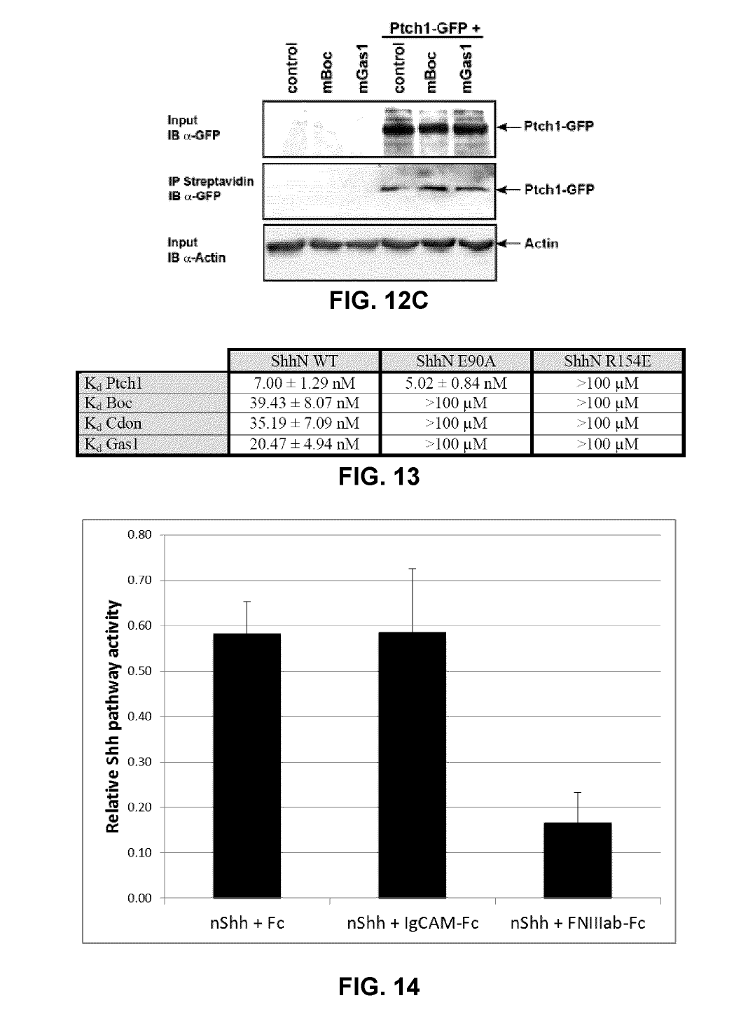

To further confirm these results, we performed the complementary experiment and looked for the presence of BOC in GAS1/PTCH1 complexes. Lysates of cells transfected with PTCH1-GFP, BOC-Flag and GAS1 were first immunoprecipitated with anti-GAS1 antibodies and, despite detecting a strong interaction between GAS1 and PTCH1 (FIG. 5F, IP#1 middle panel, lane 6), we did not detect an interaction between BOC and GAS1 in the absence nor presence of PTCH1 (FIG. 5F, top panel lanes 5-6). To confirm that BOC is indeed able to interact with PTCH1 in these lysates and test whether both BOC/PTCH1 and GAS1/PTCH1 complexes are present in the same cell lysates, we recovered the supernatants from the anti-GAS1 immunoprecipitation (IP#1) and subjected them to a second immunoprecipitation, this time with anti-Flag antibodies to immunoprecipitate BOC (FIG. 5F; see FIG. 11 for a schematic). We found that the PTCH1-GFP remaining in the supernatant efficiently co-immunoprecipitated with BOC (FIG. 5F, IP#2 middle panels, lane 6). Together, our data indicates that while BOC and GAS1 can both interact with PTCH1, it is unlikely that BOC, GAS1 and PTCH1 form a tripartite complex. Moreover, these results suggest that the BOC-PTCH1 and the GAS1-PTCH1 complexes are distinct molecular entities.

### Example 7

### Binding of Shh to PTCH1 is not Sufficient to Activate Shh Signaling

The results indicate that BOC and GAS1 are involved in Shh-mediated CGNP proliferation and that they form independent complexes with PTCH1. While BOC and GAS1 are components of these receptor complexes, they could function as partners of PTCH1, but not necessarily as receptors that bind to Shh. To determine whether the binding of Shh to GAS1 and/or BOC (and Cdon) is required for a Shh response, a mutant Shh protein unable to bind BOC/CDON/GAS1 but retaining the ability to bind PTCH1 was generated. If this mutant Shh molecule with altered specificity no longer activates signaling, it would suggest that Shh binding to BOC/CDON/GAS1 is required for pathway activation. Conversely, if this mutant form of Shh activates the pathway, it would support a model where binding to PTCH1 alone is sufficient for Shh signaling.

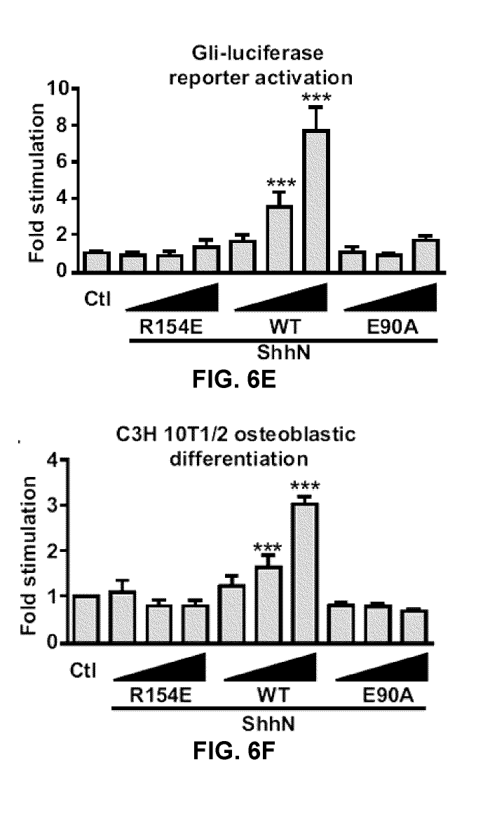

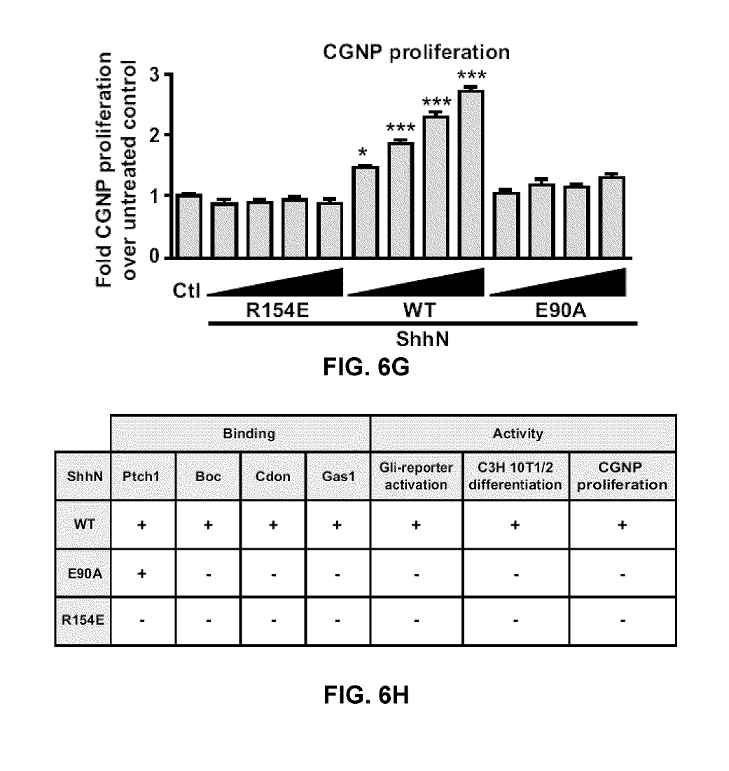

The amino acids responsible for mediating the interaction between Shh and BOC/CDON have been identified from co-crystal structures of Shh and the third FNIII domain of CDON and BOC (FIG. 6A, B) (Kavran et al., 2010; McLellan et al., 2008). Although similar structural data is unavailable for Shh in complex with PTCH1, mutagenesis of Shh surface amino acids has identified residues required and residues dispensable for the binding of Shh to PTCH1 (FIG. 6C) (Bosanac et al., 2009). Since Shh E90 is a contact amino acid between Shh and CDON/BOC (McLellan et al., 2008) that is not required for binding to (FIG. 6A,C) (Bosanac et al., 2009), we predicted that a mutation at this site might affect binding to BOC and CDON, but not to PTCH1. In contrast, Shh R154 is a contact amino acid between Shh and BOC/CDON (McLellan et al., 2008) that is also required for PTCH1 binding (FIG. 6B, C) (Bosanac et al., 2009); thus, a R154 mutation is expected to affect binding of Shh to BOC, CDON and PTCH1.

We introduced mutations of these residues into alkaline-phosphatase (AP)-tagged ShhN (ShhN-AP) and tested their binding to BOC, CDON, GAS1, and PTCH1 (FIG. 6D, H and FIG. 13). Consistent with our structural predictions, ShhN-AP R154E was unable to bind to BOC, CDON and PTCH1. Also in agreement with our predictions, ShhN-AP E90A did not bind to BOC and CDON, but retained the ability to bind PTCH1, with a dissociation constant not significantly different (p>0.05) from that of WT ShhN (FIG. 6H and FIG. 13). We also assessed the binding of our Shh mutants to GAS1 and found that they behaved similarly towards GAS1 as they did with BOC and CDON: ShhN-AP E90A and R154E were both unable to bind GAS1. Thus, according to the binding characteristics of our Shh mutants, some common amino acids may mediate the interaction of Shh with GAS1, BOC, and CDON, a finding consistent with previous reports (Kavran et al., 2010; McLellan et al., 2008)

The effect of the E90A and R154E mutations on Shh signal transduction was next examined. These mutations were introduced into untagged ShhN and recombinant proteins were purified (FIG. 6D). To measure the signaling activity of the ShhN E90A and R154E mutants, a transcription reporter assays was performed using cells stably transfected with a Gli-luciferase reporter plasmid. While WT ShhN activated Shh-mediated transcription in a concentration-dependent manner, ShhN E90A and R154E mutants were unable to do so (FIG. 6E). We next tested the ability of our Shh mutants to promote the osteoblastic differentiation of C3H 10T½ cells and neither ShhN E90A nor R154E were able to induce alkaline phosphatase expression, a marker of differentiation (FIG. 6F). Finally, we assayed the ability of these altered-specificity Shh ligands to induce CGNP proliferation. We found that while WT ShhN activated Shh-mediated proliferation in a dose-dependent manner, both ShhN E90A and R154E mutants were unable to induce proliferation (FIG. 6G). Together, these data show that ShhN E90A, which interacts with PTCH1 but not with BOC, CDON, and GAS1, fails to induce Shh signaling and Shh-dependent cellular responses. This indicates that binding of Shh to PTCH1 alone is not sufficient to activate Shh signaling, suggesting that binding to BOC, CDON, or GAS1 cell surface proteins is absolutely required for Shh-dependent signal transduction to occur.

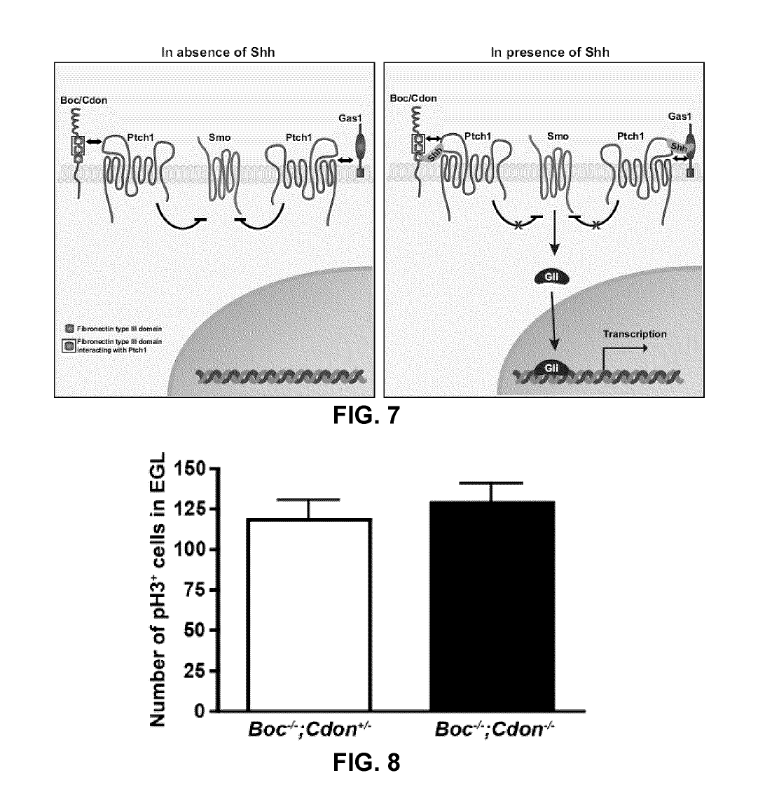

Together, these results lead us to propose the following model. BOC, CDON, and GAS1 interact with PTCH1 to form BOC/PTCH1, CDON/PTCH1 or GAS1/PTCH1 complexes (FIG. 7), with the BOC/PTCH1 interaction being mediated by the FNIIIa and FNIIIb domains of BOC. The combination of receptor complexes present in a cell would depend on the expression of BOC, CDON, and GAS1. In the presence of Shh, the ligand binds to BOC/PTCH1, CDON/PTCH1, and GAS1/PTCH1 receptor complexes, leading to the de-repression of Smo, which in turn activates a series of signal transduction events that result in Gli-mediated transcription. In the case of BOC and CDON, the interaction with Shh is direct and is mediated by the third FNIII (FNIIIc) domain of BOC and CDON (McLellan et al., 2008; Okada et al., 2006; Tenzen et al., 2006; Yao et al., 2006).

It is noteworthy that mutations of Shh amino acids important for binding to BOC, CDON, and GAS1 have been identified in holoproencephaly (HPE) and in brachydactyl)-(McLellan et al., 2008). Moreover, mutations in GAS1 have been identified in HPE patients (Ribeiro et al., 2010) and, in mouse, inactivation of Cdon and GAS1 lead to HPE (Seppala et al., 2007; Zhang et al., 2006). In addition, while Boc−/− mice do not display HPE, inactivation of Boc in a Cdon mutant background enhances the severity of the HPE phenotype in a dosage-dependent manner (Zhang et al., 2010; Zhang et al., 2006). Thus, the identification and molecular understanding of the exact components and interactions found in the vertebrate Shh receptor complexes might help our understanding of pathologies associated with defective Shh signaling. Additionally, because we show that, in addition to cell fate specification, these complexes are also involved in the control of cellular proliferation, our results provide new avenues for the treatment of Hh pathway-dependent cancers.

### Example 8

### The FNIIIab Domains of BOC Inhibit Shh Signaling

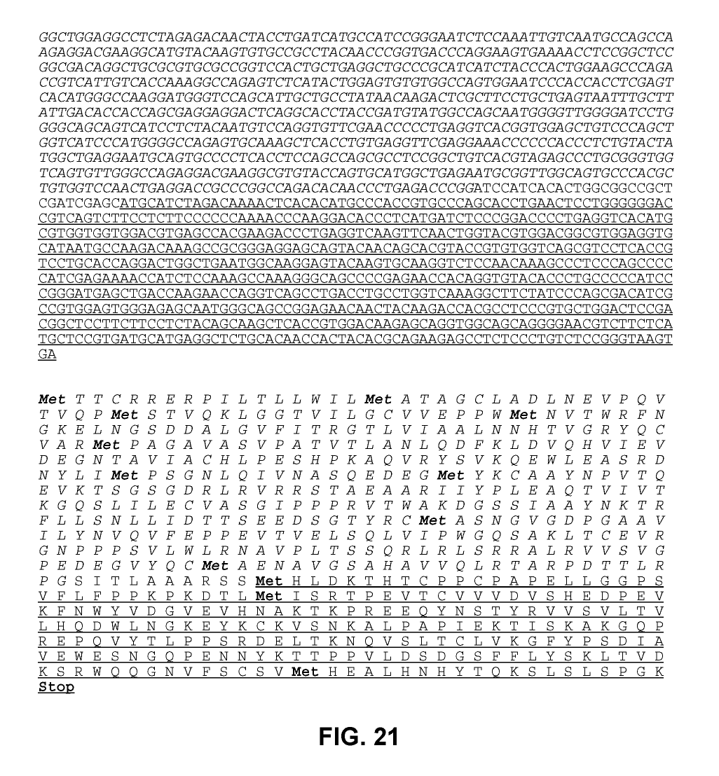

C3H 10T½ cells stably transfected with a Gli-luciferase reporter were incubated in presence of N-terminal recombinant Shh (nShh) and either Fc, IgCAM-Fc (FIG. 21, SEQ ID NOs: 11 and 12), or FNIIIab-Fc (FIG. 20, SEQ ID NOs: 9 and 10). After incubation, cells were lysed and luciferase activity (an indication of the Shh signaling pathway activity) was measured. As shown in FIG. 14, whereas the IgCAM domains of BOC had no effect on Shh-mediated activation of the Gli-luciferase reporter compared to the Fc control, the FNIIIab domains of BOC significantly inhibit Gli-luciferase reporter activation.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, an and “the” include corresponding plural references unless the context clearly dictates otherwise.

### REFERENCES

Allen, B. L., Tenzen, T., and McMahon, A. P. (2007). The Hedgehog-binding proteins Gas1 and Cdo cooperate to positively regulate Shh signaling during mouse development. Genes Dev. 21, 1244-1257.
Allen, B. L., Song, J. Y., Izzi, L., Althaus, I. W., Kang, J.-S., Charron, F., Krauss, R. S., and McMahon, A. P. (2011). Overlapping roles and collective requirement for the coreceptors GAS1, CDO, and BOC in SHH pathway function. Dev. Cell 20, 775-787.
Bosanac, I., Maun, H.R., Scales, S. J., Wen, X., Lingel, A., Bazan, J. F., de Sauvage, F. J., Hymowitz, S. G., and Lazarus, R. A. (2009). The structure of SHH in complex with HHIP reveals a recognition role for the Shh pseudo active site in signaling. Nat. Struct. Mol. Biol. 16, 691-697.
Camp, D., Currie, K., Labbe, A., van Meyel, D. J., and Charron, F. (2010). Ihog and Boi are essential for Hedgehog signaling in Drosophila. Neural Dev. 5, 28.
Charron, F., and Tessier-Lavigne, M. (2005). Novel brain wiring functions for classical morphogens: a role as graded positional cues in axon guidance. Development 132, 2251-2262.
Charron, F., Stein, E., Jeong, J., McMahon, A. P., and Tessier-Lavigne, M. (2003). The morphogen sonic hedgehog is an axonal chemoattractant that collaborates with netrin-1 in midline axon guidance. Cell 113, 11-23.
Corbit, K. C., Aanstad, P., Singla, V., Norman, A. R., Stainier, D. Y., and Reiter, J. F. (2005). Vertebrate Smoothened functions at the primary cilium. Nature 437, 1018-1021.
Corcoran, R. B., Bachar Raveh, T., Barakat, M. T., Lee, E. Y., and Scott, M. P. (2008). Insulin-like growth factor 2 is required for progression to advanced medulloblastoma in patched1 heterozygous mice. Cancer Res. 68, 8788-8795.
Corrales, J. D., Rocco, G. L., Blaess, S., Guo, Q., and Joyner, A. L. (2004). Spatial pattern of sonic hedgehog signaling through Gli genes during cerebellum development. Development 131, 5581-5590.
Dahmane, N., and Ruiz i Altaba, A. (1999). Sonic hedgehog regulates the growth and patterning of the cerebellum. Development 126, 3089-3100.
Dessaud, E., McMahon, A. P., and Briscoe, J. (2008). Pattern formation in the vertebrate neural tube: a sonic hedgehog morphogen-regulated transcriptional network. Development 135, 2489-2503.
Eggenschwiler, J. T., and Anderson, K. V. (2007). Cilia and developmental signaling. Annu. Rev. Cell Dev. Biol. 23, 345-373.
Fabre, P. J., Shimogori, T., and Charron, F. (2010). Segregation of ipsilateral retinal ganglion cell axons at the optic chiasm requires the Shh receptor Boc. J. Neurosci. 30, 266-275.
Flora, A., Klisch, T. J., Schuster, G., and Zoghbi, H. Y. (2009). Deletion of Atoh1 disrupts Sonic Hedgehog signaling in the developing cerebellum and prevents medulloblastoma. Science 326, 1424-1427.
Ingham, P. W., and Placzek, M. (2006). Orchestrating ontogenesis: variations on a theme by sonic hedgehog. Nat. Rev. Genet. 7, 841-850.
Jiang, J., and Hui, C. C. (2008). Hedgehog signaling in development and cancer. Dev. Cell 15, 801-812.
Kao, T. J., Palmesino, E., and Kania, A. (2009). SRC family kinases are required for limb trajectory selection by spinal motor axons. J Neurosci 29, 5690-5700.
Kavran, J. M., Ward, M.D., Oladosu, O. O., Mulepati, S., and Leahy, D. J. (2010). All mammalian hedgehog proteins interact with cell adhesion molecule, downregulated by oncogenes (CDO) and brother of CDO (BOC) in a conserved manner. J. Biol. Chem. 285, 24584-24590.
Kenney, A. M., and Rowitch, D. H. (2000). Sonic hedgehog promotes G(1) cyclin expression and sustained cell cycle progression in mammalian neuronal precursors. Mol. Cell. Biol. 20, 9055-9067.
Lewis, P. M., Gritli-Linde, A., Smeyne, R., Kottmann, A., and McMahon, A.P. (2004). Sonic hedgehog signaling is required for expansion of granule neuron precursors and patterning of the mouse cerebellum. Dev. Biol. 270, 393-410.
Liu, Y., May, N. R., and Fan, C. M. (2001). Growth arrest specific gene 1 is a positive growth regulator for the cerebellum. Dev. Biol. 236, 30-45.
Marigo, V., Davey, R. A., Zuo, Y., Cunningham, J. M., and Tabin, C. J. (1996). Biochemical evidence that patched is the Hedgehog receptor. Nature 384, 176-179.
Martinelli, D. C., and Fan, C. M. (2007a). Gas1 extends the range of Hedgehog action by facilitating its signaling. Genes Dev. 21, 1231-1243.
Martinelli, D. C., and Fan, C. M. (2007b). The role of Gas1 in embryonic development and its implications for human disease. Cell Cycle 6, 2650-2655.
McLellan, J. S., Yao, S., Zheng, X., Geisbrecht, B. V., Ghirlando, R., Beachy, P. A., and Leahy, D. J. (2006). Structure of a heparin-dependent complex of Hedgehog and Ihog. Proc. Natl. Acad. Sci. USA 103, 17208-17213.
McLellan, J. S., Zheng, X., Hauk, G., Ghirlando, R., Beachy, P. A., and Leahy, D. J. (2008). The mode of Hedgehog binding to Ihog homologues is not conserved across different phyla. Nature 455, 979-983.
Morin, S., Charron, F., Robitaille, L., and Nemer, M. (2000). GATA-dependent recruitment of MEF2 proteins to target promoters. EMBO J 19, 2046-2055.
Muzumdar, M. D., Tasic, B., Miyamichi, K., Li, L., and Luo, L. (2007). A global double-fluorescent Cre reporter mouse. Genesis 45, 593-605.
Okada, A., Charron, F., Morin, S., Shin, D. S., Wong, K., Fabre, P. J., Tessier-Lavigne, M., and McConnell, S. K. (2006). Boc is a receptor for sonic hedgehog in the guidance of commissural axons. Nature 444, 369-373.
Ribeiro, L. A., Quiezi, R. G., Nascimento, A., Bertolacini, C. P., and Richieri-Costa, A. (2010). Holoprosencephaly and holoprosencephaly-like phenotype and GAS1 DNA sequence changes: Report of four Brazilian patients. Am. J. Med. Genet. A 152A, 1688-1694.
Rohatgi, R., Milenkovic, L., and Scott, M. P. (2007). Patched1 regulates hedgehog signaling at the primary cilium. Science 317, 372-376.
Roussel, M. F., and Hatten, M. E. (2011). Cerebellum development and medulloblastoma. Curr. Top. Dev. Biol. 94, 235-282.
Scales, S. J., and de Sauvage, F. J. (2009). Mechanisms of Hedgehog pathway activation in cancer and implications for therapy. Trends Pharmacol. Sci. 30, 303-312.
Seppala, M., Depew, M. J., Martinelli, D. C., Fan, C. M., Sharpe, P. T., and Cobourne, M. T. (2007). Gas1 is a modifier for holoprosencephaly and genetically interacts with sonic hedgehog. J. Clin. Invest. 117, 1575-1584.
Solecki, D. J., Liu, X. L., Tomoda, T., Fang, Y., and Hatten, M. E. (2001). Activated Notch2 signaling inhibits differentiation of cerebellar granule neuron precursors by maintaining proliferation. Neuron 31, 557-568.
Tenzen, T., Allen, B. L., Cole, F., Kang, J. S., Krauss, R. S., and McMahon, A. P. (2006). The cell surface membrane proteins Cdo and Boc are components and targets of the Hedgehog signaling pathway and feedback network in mice. Dev. Cell 10, 647-656.
Traiffort, E., Dubourg, C., Faure, H., Rognan, D., Odent, S., Durou, M. R., David, V., and Ruat, M. (2004). Functional characterization of sonic hedgehog mutations associated with holoprosencephaly. J Biol Chem 279, 42889-42897.
Varjosalo and Taipale., Hedgehog: functions and mechanisms. Genes and Development 2008, 22:2454-2472.
Wallace, V. A. (1999). Purkinje-cell-derived Sonic hedgehog regulates granule neuron precursor cell proliferation in the developing mouse cerebellum. Curr. Biol. 9, 445-448.
Wechsler-Reya, R. J., and Scott, M. P. (1999). Control of neuronal precursor proliferation in the cerebellum by Sonic Hedgehog. Neuron 22, 103-114.
Yam, P. T., Langlois, S. D., Morin, S., and Charron, F. (2009). Sonic hedgehog guides axons through a noncanonical, Src-family-kinase-dependent signaling pathway. Neuron 62, 349-362.
Yao, S., Lum, L., and Beachy, P. (2006). The ihog cell-surface proteins bind Hedgehog and mediate pathway activation. Cell 125, 343-357.
Zhang, W., Kang, J. S., Cole, F., Yi, M. J., and Krauss, R. S. (2006). Cdo functions at multiple points in the Sonic Hedgehog pathway, and Cdo-deficient mice accurately model human holoprosencephaly. Dev. Cell 10, 657-665.
Zhang, W., Hong, M., Bae, G. U., Kang, J. S., and Krauss, R. S. (2011). Boc modifies the holoprosencephaly spectrum of Cdo mutant mice. Dis. Model Mech. 4, 368-380.
Zheng, X., Mann, R. K., Sever, N., and Beachy, P. A. (2010). Genetic and biochemical definition of the Hedgehog receptor. Genes Dev. 24, 57-71.

1. A method for inhibiting Sonic hedgehog (Shh)-mediated signalling and/or proliferation in a cell, said method comprising contacting said cell with an agent that inhibits the binding of a Growth Arrest Specific 1 (GAS1) polypeptide, a cell-adhesion-molecule-related/downregulated by oncogenes (CDON) polypeptide and/or biregional CDON-binding protein (BOC) polypeptide to a Patched1 (PTCH1) polypeptide, in said cell.

2-3. (canceled)

4. The method of claim 1, wherein said agent is a polypeptide comprising a sequence derived from a sequence located in a region defined by residues 45 to 317 of SEQ ID NO:6.

5. The method of claim 1, wherein said agent is an antibody directed against GAS1 that binds to an epitope located within a region defined by residues 45 to 317 of SEQ ID NO:6.

6-7. (canceled)

8. The method of claim 1, wherein said agent is a polypeptide comprising a sequence derived from a sequence located in a region defined by residues 122-436, 494-501, 569-577, 1077-1083 or 1142-1154 of SEQ ID NO:8.

9. The method of claim 1, wherein said agent is an antibody directed against PTCH1 that binds to an epitope located within a region defined by residues 122-436, 494-501, 569-577, 1077-1083 or 1142-1154 of SEQ ID NO:8.

10-12. (canceled)

13. The method of claim 1, wherein said agent is a polypeptide comprising a sequence derived from a sequence located in a region defined by residues 460 to 700 of SEQ ID NO:2.

14. The method of claim 13, wherein said agent is a polypeptide comprising a sequence derived from a sequence located in a region defined by residues 460 to 570 and/or 600 to 700 of SEQ ID NO:2.

15. The method of claim 1, wherein said agent is an antibody directed against BOC that binds to an epitope located within a region defined by residues 460 to 700 of SEQ ID NO:2.

16. (canceled)

17. The method of claim 15, wherein said antibody directed against BOC binds to an epitope within a region defined by residues 460 to 570 and/or 600 to 700 of SEQ ID NO:2.

18. (canceled)

19. The method of claim 1, wherein said agent is a polypeptide comprising a sequence derived from a sequence located in a region defined by residues 122-436, 494-501, 569-577, 1077-1083 or 1142-1154 of SEQ ID NO:8.

20. The method of claim 1, wherein said agent is an antibody directed against PTCH1 that binds to an epitope located within a region defined by residues 122-436, 494-501, 569-577, 1077-1083 or 1142-1154 of SEQ ID NO:8.

21-23. (canceled)

24. The method of claim 1, wherein said agent is a polypeptide comprising a sequence derived from a sequence located in a region defined by residues 575 to 815 of SEQ ID NO:4.

25. (canceled)

26. The method of claim 22, wherein said agent is an antibody directed against CDON that binds to an epitope located within a region defined by residues 575 to 815 of SEQ ID NO:4.

27-29. (canceled)

30. The method of claim 1, wherein said agent is a polypeptide comprising a sequence derived from a sequence located in a region defined by residues 122-436, 494-501, 569-577, 1077-1083 or 1142-1154 of SEQ ID NO:8.

31. The method of claim 1, wherein said agent is an antibody directed against PTCH1 that binds to an epitope located within a region defined by residues 122-436, 494-501, 569-577, 1077-1083 or 1142-1154 of SEQ ID NO:8.

32. (canceled)

33. The method of claim 1, wherein said cell is a tumor cell and/or a neural cell.

34-35. (canceled)

36. The method of claim 33, wherein said neural cell is a cerebellar granular neuron progenitor (CGNP).

37. A method for treating a cancer associated with GAS1, CDON, BOC and/or PTCH1 expression and/or activity in a subject, said method comprising administering to said subject an effective amount of the agent defined in claim 1.

38-39. (canceled)

40. The method of claim 37, wherein said cancer is a brain tumor, an ovary tumor, a breast tumor, a glioblastoma, a skin tumor, a meningioma, an astrocytoma, a liver tumor, a prostate carcinoma, a bladder tumor, a lung tumor, a lymph node lymphoma, a vascular endothelium hemangioma, a kidney carcinoma or a thyroid follicular adenoma.

41-51. (canceled)

52. A method of identifying an agent that may be useful for (i) inhibiting Sonic hedgehog (Shh)-mediated signalling and/or proliferation in a cell and/or (ii) treating a cancer associated with GAS1, CDON, BOC and/or PTCH1 expression and/or activity in a subject, said method comprising determining the binding of a Growth Arrest Specific 1 (GAS1), a cell-adhesion-molecule-related/downregulated by oncogenes (CDON) polypeptide and/or biregional CDON-binding protein (BOC) polypeptide to a Patched1 (PTCH1) polypeptide, in the presence of said agent, wherein a lower or decreased binding in the presence of said agent is indicative that said agent may be useful for (i) inhibiting Sonic hedgehog (Shh)-mediated signalling in a cell and/or (ii) treating a cancer associated with GAS1, CDON, BOC and/or PTCH1 expression and/or activity in a subject.

53-58. (canceled)


SEQUENCE LISTING (12 sequences)

SEQ ID NO: 1 - DNA, 4293 bp, Homo sapiens
  CDS (340)..(3684)
gtttctcatagttggcgtcttctaaaggaaaaacactaaaatgaggaactcagcggaccgggagcgacgcagcttgagggaagcatccctagctgttggcgcagaggggcgaggctgaagccgagtggcccgaggtgtctgaggggctggggcaaaggtgaaagagtttcagaacaagcttcctggaacccatgacccatgaagtcttgtcgacatttataccgtctgagggtagcagctcgaaagtagaagaagtggagtgttgccagggacggcagtatctctttgtgtgaccctggcggcttatgggacgttggcttcagacctttgtgatacaccatgctgcgtgggacgatgacggcgtggagaggaatgaggcctgaggtcacactggcttgcctcctcctagccacagcaggctgctttgctgacttgaacgaggtccctcaggtcaccgtccagcctgcgtccaccgtccagaagcccggaggcactgtgatcttgggctgcgtggtggaacctccaaggatgaatgtaacctggcgcctgaatggaaaggagctgaatggctcggatgatgctctgggtgtcctcatcacccacgggaccctcgtcatcactgcccttaacaaccacactgtgggacggtaccagtgtgtggcccggatgcctgcgggggctgtggccagcgtgccagccactgtgacactagccaatctccaggacttcaagttagatgtgcagcacgtgattgaagtggatgagggaaacacagcagtcattgcctgccacctgcctgagagccaccccaaagcccaggtccggtacagcgtcaaacaagagtggctggaggcctccagaggtaactacctgatcatgccctcagggaacctccagattgtgaatgccagccaggaggacgagggcatgtacaagtgtgcagcctacaacccagtgacccaggaagtgaaaacctccggctccagcgacaggctacgtgtgcgccgctccaccgctgaggctgcccgcatcatctaccccccagaggcccaaaccatcatcgtcaccaaaggccagagtctcattctggagtgtgtggccagtggaatcccacccccacgggtcacctgggccaaggatgggtccagtgtcaccggctacaacaagacgcgcttcctgctgagcaacctcctcatcgacaccaccagcgaggaggactcaggcacctaccgctgcatggccgacaatggggttgggcagcccggggcagcggtcatcctctacaatgtccaggtgtttgaaccccctgaggtcaccatggagctatcccagctggtcatcccctggggccagagtgccaagcttacctgtgaggtgcgtgggaaccccccgccctccgtgctgtggctgaggaatgctgtgcccctcatctccagccagcgcctccggctctcccgcagggccctgcgcgtgctcagcatggggcctgaggacgaaggcgtctaccagtgcatggccgagaacgaggttgggagcgcccatgccgtagtccagctgcggacctccaggccaagcataaccccaaggctatggcaggatgctgagctggctactggcacacctcctgtatcaccctccaaactcggcaaccctgagcagatgctgagggggcaaccggcgctccccagacccccaacgtcagtggggcctgcttccccgcagtgtccaggagagaaggggcagggggctcccgccgaggctcccatcatcctcagctcgccccgcacctccaagacagactcatatgaactggtgtggcggcctcggcatgagggcagtggccgggcgccaatcctctactatgtggtgaaacaccgcaaggtcacaaattcctctgacgattggaccatctctggcattccagccaaccagcaccgcctgaccctcaccagacttgaccccgggagcttgtatgaagtggagatggcagcttacaactgtgcgggagagggccagacagccatggtcaccttccgaactggacggcggcccaaacccgagatcatggccagcaaagagcagcagatccagagagacgaccctggagccagtccccagagcagcagccagccagaccacggccgcctctcccccccagaagctcccgacaggcccaccatctccacggcctccgagacctcagtgtacgtgacctggattccccgtgggaatggtgggttcccaatccagtccttccgtgtggagtacaagaagctaaagaaagtgggagactggattctggccaccagcgccatccccccatcgcggctgtccgtggagatcacgggcctagagaaaggcacctcctacaagtttcgagtccgggctctgaacatgctgggggagagcgagcccagcgccccctctcggccctacgtggtgtcgggctacagcggtcgcgtgtacgagaggcccgtggcaggtccttatatcaccttcacggatgcggtcaatgagaccaccatcatgctcaagtggatgtacatcccagcaagtaacaacaacaccccaatccatggcttttatatctattatcgacccacagacagtgacaatgatagtgactacaagaaggatatggtggaaggggacaagtactggcactccatcagccacctgcagccagagacctcctacgacattaagatgcagtgcttcaatgaaggaggggagagcgagttcagcaacgtgatgatctgtgagaccaaagctcggaagtcttctggccagcctggtcgactgccacccccaactctggccccaccacagccgccccttcctgaaaccatagagcggccggtgggcactggggccatggtggctcgctccagcgacctgccctatctgattgtcggggtcgtcctgggctccatcgttctcatcatcgtcaccttcatccccttctgcttgtggagggcctggtctaagcaaaaacatacaacagacctgggttttcctcgaagtgcccttccaccctcctgcccgtatactatggtgccattgggaggactcccaggccaccaggccagtggacagccctacctcagtggcatcagtggacgggcctgtgctaatgggatccacatgaataggggctgcccctcggctgcagtgggctacccgggcatgaagccccagcagcactgcccaggcgagcttcagcagcagagtgacaccagcagcctgctgaggcagacccatcttggcaatggatatgacccccaaagtcaccagatcacgaggggtcccaagtctagcccggacgagggctctttcttatacacactgcccgacgactccactcaccagctgctgcagccccatcacgactgctgccaacgccaggagcagcctgctgctgtgggccagtcaggggtgaggagagcccccgacagtcctgtcctggaagcagtgtgggaccctccatttcactcagggcccccatgctgcttgggccttgtgccagttgaagaggtggacagtcctgactcctgccaagtgagtggaggagactggtgtccccagcaccccgtaggggcctacgtaggacaggaacctggaatgcagctctccccggggccactggtgcgtgtgtcttttgaaacaccacctctcacaatttaggcagaagctgatatcccagaaagactatatattgttttttttttaaaaaaaaaaagaagaaaaaagagacagagaaaattggtatttatttttctattatagccatatttatatatttatgcacttgtaaataaatgtatatgttttataattctggagagacataaggagtcctacccgttgaggttggagagggaaaataaagaagctgccacctaacaggagtcacccaggaaagcaccgcacaggctggcgcgggacagactcctaacctggggcctctgcagtggcaggcgaggctgcaggaggcccacagataagctggcaagaggaaggatcccaggcacatggttcatcacgagcatgagggaacagcaaggggcacggtatcacagcctggagacacccacacagatggctggatccggtgctacgggaaacattttcctaagatgcccatgagaacagaccaagatgtgtacagcactatgagcattaaaaaaccttccagaatcaataatccgtggcaacatatctctgtaaaaacaaacactgtaacttctaaataaatgtttagtcttccctgtaaccttcaaaaaaaaaaaaaaa

SEQ ID NO: 2 - protein, 1114 aa, Homo sapiens
MLRGTMTAWRGMRPEVTLACLLLATAGCFADLNEVPQVTVQPASTVQKPGGTVILGCVVEPPRMNVTWRLNGKELNGSDDALGVLITHGTLVITALNNHTVGRYQCVARMPAGAVASVPATVTLANLQDFKLDVQHVIEVDEGNTAVIACHLPESHPKAQVRYSVKQEWLEASRGNYLIMPSGNLQIVNASQEDEGMYKCAAYNPVTQEVKTSGSSDRLRVRRSTAEAARIIYPPEAQTIIVTKGQSLILECVASGIPPPRVTWAKDGSSVTGYNKTRFLLSNLLIDTTSEEDSGTYRCMADNGVGQPGAAVILYNVQVFEPPEVTMELSQLVIPWGQSAKLTCEVRGNPPPSVLWLRNAVPLISSQRLRLSRRALRVLSMGPEDEGVYQCMAENEVGSAHAVVQLRTSRPSITPRLWQDAELATGTPPVSPSKLGNPEQMLRGQPALPRPPTSVGPASPQCPGEKGQGAPAEAPIILSSPRTSKTDSYELVWRPRHEGSGRAPILYYVVKHRKVTNSSDDWTISGIPANQHRLTLTRLDPGSLYEVEMAAYNCAGEGQTAMVTFRTGRRPKPEIMASKEQQIQRDDPGASPQSSSQPDHGRLSPPEAPDRPTISTASETSVYVTWIPRGNGGFPIQSFRVEYKKLKKVGDWILATSAIPPSRLSVEITGLEKGTSYKFRVRALNMLGESEPSAPSRPYVVSGYSGRVYERPVAGPYITFTDAVNETTIMLKWMYIPASNNNTPIHGFYIYYRPTDSDNDSDYKKDMVEGDKYWHSISHLQPETSYDIKMQCFNEGGESEFSNVMICETKARKSSGQPGRLPPPTLAPPQPPLPETIERPVGTGAMVARSSDLPYLIVGVVLGSIVLIIVTFIPFCLWRAWSKQKHTTDLGFPRSALPPSCPYTMVPLGGLPGHQASGQPYLSGISGRACANGIHMNRGCPSAAVGYPGMKPQQHCPGELQQQSDTSSLLRQTHLGNGYDPQSHQITRGPKSSPDEGSFLYTLPDDSTHQLLQPHHDCCQRQEQPAAVGQSGVRRAPDSPVLEAVWDPPFHSGPPCCLGLVPVEEVDSPDSCQVSGGDWCPQHPVGAYVGQEPGMQLSPGPLVRVSFETPPLTI

SEQ ID NO: 3 - DNA, 8116 bp, Homo sapiens
  CDS (129)..(3992)
gtctctccaagggaatgaaaggtgaggggagcgtgagtgcaggcatgatgggaatctccgtgacttgtgaaccaaggactgggttttgctctggaagcctgtcctgattgcttctgtaatctggcgctatgcatccggatcttggacccttatgtacactgctgtatgttactcttacaattctgtgctcttctgtgagttcagacttggcaccttattttacttctgagccgctctctgctgtccagaaacttggtggacctgtagtactgcattgttctgctcaacctgtgaccactcgtatctcatggctgcataacggaaaaacattggatggaaacctggaacatgttaagattcatcaggggactctgacaattctttctctcaactcctctcttttgggttactaccagtgccttgccaacaatagcatcggtgccattgtgagtggccctgcgacagtatctgtggcagttcttggtgattttggttcatccacaaagcatgttattacagcagaagaaaaaagtgctggtttcattggctgcagggtaccggagagtaaccccaaagctgaggtgcgctataaaatccggggaaaatggctggaacattccacagagaattacttaatccttccatcaggaaatcttcagattttgaatgtatccttagaggacaagggatcatacaaatgtgcagcttataatcctgtcacacatcaattaaaagttgaacctattggccgaaagctccttgtgagtcgtccttcttcagatgatgttcacattcttcaccccacccattcacaggcattagctgttctttctcgtagccctgtaaccttggagtgtgtggtgagtggggtcccggctcctcaagtgtattggctaaaggacgggcaggacattgcaccaggaagcaactggagaaggttgtattctcatcttgccactgatagcgttgacccggcggactccggaaactattcctgcatggcgggaaacaagtctggagatgtaaaatatgtgacttacatggttaatgtacttgaacatgcttccatttctaaaggactacaggatcagatagtgtctctgggtgccacagtacactttacctgcgacgttcatgggaacccagcccccaactgtacctggtttcacaatgcacagcctattcatccttctgcacgacatctaactgcaggaaacggactgaaaatcagtggggttactgtggaagatgttgggatgtatcagtgtgtagcagataatgggattggatttatgcactctactggaagacttgaaattgaaaatgacggtggattcaagccagttataattacggcaccagtaagtgcaaaggttgcagacggagactttgttactctgtcctgcaatgccagtgggctgccggttccggtcattcgttggtatgacagccatggattgataaccagccatccatctcaagtcctgagatcgaaatcccgaaaatcacagttatcaagacctgagggcttgaacctggagcctgtgtacttcgtcctgtcccaagctggtgcaagctctctccatattcaggctgtgactcaggaacatgcggggaaatacatctgcgaagctgcaaatgaacatggtaccacacaggcagaagcatctctcatggttgttccttttgaaacaaatacaaaagcagagacagtcacacttcctgatgctgctcagaatgatgacagaagtaagagagatggttcagaaactgggttactgagctcatttccggtgaaggtccatcccagtgcagtggaatcagcaccagagaaaaacgccagcggcatctctgttcctgatgcccccatcatactgagccccccacagacccacacaccagacacgtacaacctggtgtggagggcaggcaaggatggtgggctgcccatcaatgcttactttgtgaagtatcgaaagctggatgatggggttggcatgctgggaagctggcacacggttcgagtcccaggaagtgaaaatgagctccatttagctgagctggagccatctagtctttatgaagtcttgatggtagcaagaagcgcagcaggtgaaggccaacctgccatgcttaccttccgaaccagcaaagaaaaaacagcgtcatcaaaaaacacccaggcatcctctccacccgtgggcatccctaagtatcccgttgtttcagaggctgcaaacaacaattttggagtggtacttacagattcctctaggcacagtggagttccagaggcaccagatcggcctaccatctccactgcatcagagacatcagtctatgtcacttggattcctcgggcaaacgggggttctccaatcactgccttcaaagtcgaatataaacggatgaggaccagcaattggctggtggcagctgaagacatccctccttccaaactttcagtggaagttcgtagtttagaaccaggttcaacatacaaatttagggtcattgccatcaaccattatggtgagagttttcggagttcagcatctcgtccttatcaagtggttgggttccccaatcgcttttccagccgtccaataactggacctcacattgcatacacagaggctgtcagcgatactcagatcatgctaaagtggacgtacattccatcaagtaacaataacactcccattcaaggattttatatctattaccgaccaacagatagtgacaatgacagtgattacaagagggatgttgtagaaggttcaaagcagtggcacatgattggccacctgcagccagaaacctcctatgacattaaaatgcaatgcttcaatgaaggaggagaaagtgaatttagcaatgtgatgatctgcgagactaaagtgaaacgtgttcctggagcttctgaatatcctgtcaaagacttgagtacccctccaaattctttgggaagtggaggaaatgtggggcctgcaaccagccctgccagaagcagtgacatgttatatctgatcgttggctgtgtgctgggcgtcatggtcctcattctgatggttttcattgcaatgtgcctgtggaagaatcgccagcagaataccatacaaaaatatgacccaccaggatatctctaccaaggatcagatatgaacgggcagatggtggactacaccactctctcaggagcaagtcagataaatggaaatgttcacggaggcttcctaaccaatggcggtctcagcagtggctattcccaccttcaccataaggtccccaatgcagtcaatggaattgtgaatgggagcctaaatggagggctttactccgggcacagcaactctctaaccaggacacacgtggattttgaacatcctcatcatctagtgaatggtggtggaatgtacacggccgtgcctcagattgaccctctggagtgtgttaactgccgaaattgtcgaaacaacaataggtgtttcaccaaaaccaacagcactttcagcagcagccctcctcctgtggtccctgtggtagcaccttatcctcaggatggtttggaaatgaagcccctcagtcacgtgaaggtgcctgtatgcctgacttccgcagtccctgattgtggccagttgccggaggagagcgtcaaggacaatgtggaaccagtccctactcagcgtacctgctgtcaggacattgtaaatgacgtcagctctgatggctcagaagatccagcagagttcagcagaggtcaggaaggcatgatcaaccttaggattccagatcatcttcagttagctaagagctgtgtatgggaaggagacagctgtgcccattcagaaacagagatcaacattgtaagttggaatgctcttattttgccacctgtccccgagggctgtgctgagaagacaatgtggtctccacctggcattcctttagacagcccgacagaggtccttcagcagccccgggaaacctgaggacatgcaaacaaccagtcatgttccaacttcaagccggtaactgcacacaacaggcctgggagcgaactgtgtgaaggaccttaattcaaatcagagaaaatcattatttatttttttgtagtagtaatgtcatatgaatgtatcttaaaacgtgtgcccttttatattatttatgccttaaatgttttcttccccattccttcctccccctcggtaggaaacaaccttgttttgcatagtattcagtcacctggagggcagaggggtccttccatgttttctaacagctatagtgacagcaagagctcctcatgcaaaggatcccatctcttggctgcttcggctaggagggaatgcaggggcccgttgaaggttgccaccagctgggttttcaggtggaaaatggtctttcaatagtgtattctcagacttttttttgtttgttttttgagatggagtcttgctgtcatccaggctggagtgcaatggctcgatcttggctcactgcaacctccgcctcccaggttcaagcaattctcccgcctcagcctcctgagtagctgggactacaggcacctgccaccacgtccagctaattgtcttgtatttttattagagacaaggtttcaggctggttttgaactcctgacctcaagtgatccacccgcctccgcctcccaaagtgcgaggattacaggcgtgagccaccacgtctggcctgtattatcagacttcctgagaacactttgaaatcaactgcagttgtgacccatgtttataataacagacctggctgacggattttacaagagtgccttccacagaagtggtaatgtgacctgttgactttgtatctgcacgtgggcacgagtgatgttcagtttgctaggcactagtcatagtgttatggacgtggtggagtgtaaggtcttgatgaattacggcagtatgcagaaaaggaaccaaggccagagagacaaataatgcctcatgtcccactgctttaaaattacattaatttataaaatggccactatgggctctttttgactgtttctcggagtaggaacaaaataagacattaaatggtggcttgaagagaaagatacacattttcagaagaaagaaaggggagggctgcagggagcctgtcttggcgggagctctccagtctgttggattagcacagggacacgcttatggtgccatgacgccgaaactagtcatcccccatctccagcataggcaacggcctgcaggggtgagtgtcagaaaagacttactttggaagaaaagggtttttttttgtttgtttgttttttgtttttttttgtttgtttgttttgggtttttttcctgaaatttcctgatacctttttcagaatgtcacccttgtaaataggcacctaaagccaagttggtcagcagagctcctgaccacggctgcctctgcgtgacaaggacatttgcagctgcctttggaaggctcttcacattaaaaaacaataattatgtaagtgtgtttcatagcttgagtcaagctttcaaggatcactgctcctttgtgcattgtgcatccctttggggaaagaaacagaaaattattctctttctatagagtcaagcaattccagcagaagtgaagaaatgttagtttcccagaaatcgcagtggaatagaatacaatactgtgttaaccaactttctgtataaatgcatcagtattcccttacatatgtctatatattttataatagaactgttagtgaaaatgtgcttgtttaagctgggtcactatgcaagagttacttccagtagaatgtagtgaatgtaattccctagtcggatattagggatgctgtagacccaagttctacattccagatgtccgtgcctatagtatactggatttattcttcatgttgcttaaatgcagttagctctggagggggatagctgcatcccttacttctgtgactagcaaaggacaaagtcccagcgcacagaatatgaaaacataccctgtgttagccaatcaagttacagcaatgctgtcacaaagggagttttagaaaactggggagtcattgtccgtagacagccattttgatttaggaaacaggtgtattagcatgagccttaggtcatgaactgcttttaaagattgagtttgaatatgggatctgtccacgaggaagagaaaatggctttacctttaggacaatctcattgtggactgctttatttatattattagtaacgctgttttaagtgttattctttttattgtattttatttataaaaaatgcctttatatattgaaatatataaatatatatatataaacacacacacacacacacacacacacatatatatatatatatataattgcctttatatttaggcggtatgagttctccctaagcgtgagttggctctgccctgcacaggcacccaaacagtcctacctaatgtcacttcaccaggaaatgagaggacatggtacagtgtgggagacagcactaacacgtgctgaataactactgtgtaccaggcactgtgctcggtgcttgttactgttaactcatttaacccccacaactcatctacaaagttggcattaaaatccccaagtcactcagtgagccatgggacctgaatccgaacctagtttatctggagccataatctgggctcctcccatttttccatactgctttccaaaagcattgtggttcactctttggatggaaccagtcccgttccgtatgctgtgatctatccctgccgtggaattcctccgaggctccaactccgcagccagagaagctgctctctggcctccgcgggcagagctctgcttatgacttcgtcttccagaggtccacatttctccccttgacgccccctgtctaccacctccacctggaaatttgatctttactgtgctcagtgatcacgaaattggtcaagcctattcatgttccttctcatctagaattctgtgtgattgttttagcataaactgccccgctcccactgtatcatttcacgatctccttttaatgtttttctttcagttttgcactgacagtctggtgacaggtgctgccagggtgcaagttacattgcaatgttctaaaccttttgaagtgttcttagcaaaagcctttctttctgggtgtttttgtcgaatgagtctatgaagactcaatgcatccatcttgggctgatcatgccacagatctcattccagctatagtactgccaagaagtgaagtttatcattgaattttacctgtgttgctctcttttaaagtaatgactttgagatgcatgctttgtatcatattgaaaagtatgttttattttccttaaagttgtaatattgtgtgtgaaactgtacatattttaaatgatgtagctttggcaggttttatttggttgatatgattttatattaaaagagaaggctgctttcatatagcatagcccctgaaggtgggaatactccctgtttgtacaacgctgcttatagtttgtgattggggtgggcgaatgtcttcctctgaaattctttggaaccccccacccctgtcctcccctgaaactcccctgtgcacacccctctgttctgactgcagagatcaaggccctgaaggagcaggatcttgggcaagtgttttaggggacgctgagaacatacctgaccctgggggcgatgtggaggcaggcccacctcctcggggcaactttgcgtttccttggagggaagggaggtgggtaggagaggagcttgggaggatgaggaggagcggtcccgcctcacacattgtctatttttaccattaaccgagcgccacatgtttatgaacttcaggatagaaatttagcctccttgtgaaaacgttttgttttactgccctaagaaaatacacatctctaggcttgtctgaccagaaagccagcaaagtcttttgcatcgggtgtggcccttttgtattttatttcccatatttaaataagccttccttaaaccctagccccagtcctgagagctttgagggtagccgaaaccccttatctaataaagtctaactctgtgagaaccacagaa

SEQ ID NO: 4 - protein, 1287 aa, Homo sapiens
MHPDLGPLCTLLYVTLTILCSSVSSDLAPYFTSEPLSAVQKLGGPVVLHCSAQPVTTRISWLHNGKTLDGNLEHVKIHQGTLTILSLNSSLLGYYQCLANNSIGAIVSGPATVSVAVLGDFGSSTKHVITAEEKSAGFIGCRVPESNPKAEVRYKIRGKWLEHSTENYLILPSGNLQILNVSLEDKGSYKCAAYNPVTHQLKVEPIGRKLLVSRPSSDDVHILHPTHSQALAVLSRSPVTLECVVSGVPAPQVYWLKDGQDIAPGSNWRRLYSHLATDSVDPADSGNYSCMAGNKSGDVKYVTYMVNVLEHASISKGLQDQIVSLGATVHFTCDVHGNPAPNCTWFHNAQPIHPSARHLTAGNGLKISGVTVEDVGMYQCVADNGIGFMHSTGRLEIENDGGFKPVIITAPVSAKVADGDFVTLSCNASGLPVPVIRWYDSHGLITSHPSQVLRSKSRKSQLSRPEGLNLEPVYFVLSQAGASSLHIQAVTQEHAGKYICEAANEHGTTQAEASLMVVPFETNTKAETVTLPDAAQNDDRSKRDGSETGLLSSFPVKVHPSAVESAPEKNASGISVPDAPIILSPPQTHTPDTYNLVWRAGKDGGLPINAYFVKYRKLDDGVGMLGSWHTVRVPGSENELHLAELEPSSLYEVLMVARSAAGEGQPAMLTFRTSKEKTASSKNTQASSPPVGIPKYPVVSEAANNNFGVVLTDSSRHSGVPEAPDRPTISTASETSVYVTWIPRANGGSPITAFKVEYKRMRTSNWLVAAEDIPPSKLSVEVRSLEPGSTYKFRVIAINHYGESFRSSASRPYQVVGFPNRFSSRPITGPHIAYTEAVSDTQIMLKWTYIPSSNNNTPIQGFYIYYRPTDSDNDSDYKRDVVEGSKQWHMIGHLQPETSYDIKMQCFNEGGESEFSNVMICETKVKRVPGASEYPVKDLSTPPNSLGSGGNVGPATSPARSSDMLYLIVGCVLGVMVLILMVFIAMCLWKNRQQNTIQKYDPPGYLYQGSDMNGQMVDYTTLSGASQINGNVHGGFLTNGGLSSGYSHLHHKVPNAVNGIVNGSLNGGLYSGHSNSLTRTHVDFEHPHHLVNGGGMYTAVPQIDPLECVNCRNCRNNNRCFTKTNSTFSSSPPPVVPVVAPYPQDGLEMKPLSHVKVPVCLTSAVPDCGQLPEESVKDNVEPVPTQRTCCQDIVNDVSSDGSEDPAEFSRGQEGMINLRIPDHLQLAKSCVWEGDSCAHSETEINIVSWNALILPPVPEGCAEKTMWSPPGIPLDSPTEVLQQPRET

SEQ ID NO: 5 - DNA, 2844 bp, Homo sapiens
  CDS (411)..(1448)
agcagccggcacggggacagccggccgcacaacggatctgcaggcgcggagcaaaatgcacccgccgcgccgcgcggtcctgcagccccgccacggccccgcggcccgcacccccccggggcgacagtgagcctctcccgccaccaccgggggccgagcggagggctctcgggtgggagagcgggaccagatctcgacagctgttcatttccaggaagccaccgcagccagagcgaaaggggaccttctgccaccagcggggcatcagccagcggcgcgcatggatttatgaagacactcatgcaagaagtgggcaggacttggacaaacttttccaccggctccgcgtccgccgctccccgcgcctcgtctcctttcccctcctctcccggcggccgccgctgcccgcgatggtggccgcgctgctgggcggcggcggcgaggcccgcggggggacagtgccgggcgcctggctgtgcctgatggcgctgctgcagctgctgggctcggcgccgcggggatcggggctggcgcacggccgccgcctcatctgctggcaggcgctgctgcagtgccagggggagccggagtgcagctacgcctacaaccagtacgccgaggcgtgcgcgccggtgctggcgcagcacggcgggggcgacgcgcccggggccgccgccgccgctttcccggcctcggccgcctctttctcgtcgcgctggcgctgcccgagtcactgcatctcggccctcattcagctcaaccacacgcgccgcgggcccgccctggaggactgtgactgcgcgcaggacgagaactgcaagtccaccaagcgcgccattgagccgtgcctgccccggacgagcggcggcggcgcgggcggccccggcgcgggcggggtcatgggctgcaccgaggcccggcggcgctgcgaccgcgacagccgctgcaacctggcgctgagccgctacctgacctactgcggcaaagtcttcaacgggctgcgctgcacggacgaatgccgcaccgtcattgaggacatgctggctatgcccaaggcggcgctgctcaacgactgcgtgtgcgacggcctcgagcggcccatctgcgagtcggtcaaggagaacatggcccgcctgtgcttcggcgccgagctgggcaacggccccggcagcagcggctcggacgggggcctggacgactactacgatgaggactacgatgacgagcagcgcaccgggggcgcgggtggtgagcagccgctggacgacgacgacggcgtcccgcacccaccgcgcccgggcagcggcgctgctgcatcgggcggccgcggggacctgccctatgggcctgggcgcaggagcagcggcggcggcggccgcttggcgccccggggcgcctggaccccactcgcctccatcttgctgctgctgcttgggccgctcttttagccctcgcgccccccgccgttggctgcgggagagcccgcgtcccactcccgtgctcgcctcgaccccgcgccgggcacctgtggcttgggacagatagaagggatggttggggatacttcccaaaactttttccaagtcaacttggtgtagccggttccccggccacgactctgggcacttcccctgaagctcctctccggagcttgacttcttggacctcctcccccgccccaattccaagctccagaaactcccaactcgtctgccgtccagaaagctagctgcagtgttcaggacgtccgggaggaagcaagcatgtgggggacagaacagtagtcctggactcgaaagggaaggtgctgaccagtggggccttagcaatttgaagggttgggaaggaggaattatatttgcaaaggggctgtctattagcatatttcctttgagggggcaaaaaaaagtgccagtatcgacttttacagattgtggccagtgaggatattataatcctatgtaaacagaaaagtcccacttaccgattcattctttcactgtttgtatctgcgcccagaattctcagtgacgtgggggtgagggtgggtggcgattgccttagagggaacccctaaattggttttggataagtttgagcccttgaccttaatttcattgctaccactctgatctcttagcacatttcttaggattaagggtccaaaaatgctgatctaaggggttgccatggtgttgaacaatgcaactttttatttaaaaaagctctgcactgccatgtatgaaagtctctttatgatgtttgtttttttgtcatttttgttctttacatcaagaaattttatgtttaaatatgcggagaatgtatattgcctctgctcctatcagggttgctaaaccctggtacatcgtatataaaatgtattaaaactggggtttgttaccagttgctgtactttgtatatagaatttttataaattgtatgcttcagaaataatttatttttaaaaagaaattaaaagttttaaactcacatccatattacacctttcccccctgaaatgtatagaatccatttgtcatcaggaatcaaaacccacagtccattgtgaagtgtgctatatttagaacagtcttaaaatgtacagtgtattttatagaattgaagttaacattcttattttcaagagaatttatggacgttgtagaaatgtacaaatgcatttccaaactgccttaaacgttgtatttttatagacatgttttttaaaaatcctaagtttttaaataactatggatttgtgtattttttttggttatttgttttattaaaacatgtacatcagtaaagagttttaaacaatgaaaaaaaaaaaaaaaaaa

SEQ ID NO: 6 - protein, 345 aa, Homo sapiens
MVAALLGGGGEARGGTVPGAWLCLMALLQLLGSAPRGSGLAHGRRLICWQALLQCQGEPECSYAYNQYAEACAPVLAQHGGGDAPGAAAAAFPASAASFSSRWRCPSHCISALIQLNHTRRGPALEDCDCAQDENCKSTKRAIEPCLPRTSGGGAGGPGAGGVMGCTEARRRCDRDSRCNLALSRYLTYCGKVFNGLRCTDECRTVIEDMLAMPKAALLNDCVCDGLERPICESVKENMARLCFGAELGNGPGSSGSDGGLDDYYDEDYDDEQRTGGAGGEQPLDDDDGVPHPPRPGSGAAASGGRGDLPYGPGRRSSGGGGRLAPRGAWTPLASILLLLLGPLF

SEQ ID NO: 7 - DNA, 7897 bp, Homo sapiens
  CDS (189)..(4532)
gcgcccgccgtgtgagcagcagcagcggctggtctgtcaaccggagcccgagcccgagcagcctgcggccagcagcgtcctcgcaagccgagcgcccaggcgcgccaggagcccgcagcagcggcagcagcgcgccgggccgcccgggaagcctccgtccccgcggcggcggcggcggcggcggcaacatggcctcggctggtaacgccgccgagccccaggaccgcggcggcggcggcagcggctgtatcggtgccccgggacggccggctggaggcgggaggcgcagacggacgggggggctgcgccgtgctgccgcgccggaccgggactatctgcaccggcccagctactgcgacgccgccttcgctctggagcagatttccaaggggaaggctactggccggaaagcgccgctgtggctgagagcgaagtttcagagactcttatttaaactgggttgttacattcaaaaaaactgcggcaagttcttggttgtgggcctcctcatatttggggccttcgcggtgggattaaaagcagcgaacctcgagaccaacgtggaggagctgtgggtggaagttggaggacgagtaagtcgtgaattaaattatactcgccagaagattggagaagaggctatgtttaatcctcaactcatgatacagacccctaaagaagaaggtgctaatgtcctgaccacagaagcgctcctacaacacctggactcggcactccaggccagccgtgtccatgtatacatgtacaacaggcagtggaaattggaacatttgtgttacaaatcaggagagcttatcacagaaacaggttacatggatcagataatagaatatctttacccttgtttgattattacacctttggactgcttctgggaaggggcgaaattacagtctgggacagcatacctcctaggtaaacctcctttgcggtggacaaacttcgaccctttggaattcctggaagagttaaagaaaataaactatcaagtggacagctgggaggaaatgctgaataaggctgaggttggtcatggttacatggaccgcccctgcctcaatccggccgatccagactgccccgccacagcccccaacaaaaattcaaccaaacctcttgatatggcccttgttttgaatggtggatgtcatggcttatccagaaagtatatgcactggcaggaggagttgattgtgggtggcacagtcaagaacagcactggaaaactcgtcagcgcccatgccctgcagaccatgttccagttaatgactcccaagcaaatgtacgagcacttcaaggggtacgagtatgtctcacacatcaactggaacgaggacaaagcggcagccatcctggaggcctggcagaggacatatgtggaggtggttcatcagagtgtcgcacagaactccactcaaaaggtgctttccttcaccaccacgaccctggacgacatcctgaaatccttctctgacgtcagtgtcatccgcgtggccagcggctacttactcatgctcgcctatgcctgtctaaccatgctgcgctgggactgctccaagtcccagggtgccgtggggctggctggcgtcctgctggttgcactgtcagtggctgcaggactgggcctgtgctcattgatcggaatttcctttaacgctgcaacaactcaggttttgccatttctcgctcttggtgttggtgtggatgatgtttttcttctggcccacgccttcagtgaaacaggacagaataaaagaatcccttttgaggacaggaccggggagtgcctgaagcgcacaggagccagcgtggccctcacgtccatcagcaatgtcacagccttcttcatggccgcgttaatcccaattcccgctctgcgggcgttctccctccaggcagcggtagtagtggtgttcaattttgccatggttctgctcatttttcctgcaattctcagcatggatttatatcgacgcgaggacaggagactggatattttctgctgttttacaagcccctgcgtcagcagagtgattcaggttgaacctcaggcctacaccgacacacacgacaatacccgctacagccccccacctccctacagcagccacagctttgcccatgaaacgcagattaccatgcagtccactgtccagctccgcacggagtacgacccccacacgcacgtgtactacaccaccgctgagccgcgctccgagatctctgtgcagcccgtcaccgtgacacaggacaccctcagctgccagagcccagagagcaccagctccacaagggacctgctctcccagttctccgactccagcctccactgcctcgagcccccctgtacgaagtggacactctcatcttttgctgagaagcactatgctcctttcctcttgaaaccaaaagccaaggtagtggtgatcttcctttttctgggcttgctgggggtcagcctttatggcaccacccgagtgagagacgggctggaccttacggacattgtacctcgggaaaccagagaatatgactttattgctgcacaattcaaatacttttctttctacaacatgtatatagtcacccagaaagcagactacccgaatatccagcacttactttacgacctacacaggagtttcagtaacgtgaagtatgtcatgttggaagaaaacaaacagcttcccaaaatgtggctgcactacttcagagactggcttcagggacttcaggatgcatttgacagtgactgggaaaccgggaaaatcatgccaaacaattacaagaatggatcagacgatggagtccttgcctacaaactcctggtgcaaaccggcagccgcgataagcccatcgacatcagccagttgactaaacagcgtctggtggatgcagatggcatcattaatcccagcgctttctacatctacctgacggcttgggtcagcaacgaccccgtcgcgtatgctgcctcccaggccaacatccggccacaccgaccagaatgggtccacgacaaagccgactacatgcctgaaacaaggctgagaatcccggcagcagagcccatcgagtatgcccagttccctttctacctcaacggcttgcgggacacctcagactttgtggaggcaattgaaaaagtaaggaccatctgcagcaactatacgagcctggggctgtccagttaccccaacggctaccccttcctcttctgggagcagtacatcggcctccgccactggctgctgctgttcatcagcgtggtgttggcctgcacattcctcgtgtgcgctgtcttccttctgaacccctggacggccgggatcattgtgatggtcctggcgctgatgacggtcgagctgttcggcatgatgggcctcatcggaatcaagctcagtgccgtgcccgtggtcatcctgatcgcttctgttggcataggagtggagttcaccgttcacgttgctttggcctttctgacggccatcggcgacaagaaccgcagggctgtgcttgccctggagcacatgtttgcacccgtcctggatggcgccgtgtccactctgctgggagtgctgatgctggcgggatctgagttcgacttcattgtcaggtatttctttgctgtgctggcgatcctcaccatcctcggcgttctcaatgggctggttttgcttcccgtgcttttgtctttctttggaccatatcctgaggtgtctccagccaacggcttgaaccgcctgcccacaccctcccctgagccaccccccagcgtggtccgcttcgccatgccgcccggccacacgcacagcgggtctgattcctccgactcggagtatagttcccagacgacagtgtcaggcctcagcgaggagcttcggcactacgaggcccagcagggcgcgggaggccctgcccaccaagtgatcgtggaagccacagaaaaccccgtcttcgcccactccactgtggtccatcccgaatccaggcatcacccaccctcgaacccgagacagcagccccacctggactcagggtccctgcctcccggacggcaaggccagcagccccgcagggacccccccagagaaggcttgtggccacccccctacagaccgcgcagagacgcttttgaaatttctactgaagggcattctggccctagcaatagggcccgctggggccctcgcggggcccgttctcacaaccctcggaacccagcgtccactgccatgggcagctccgtgcccggctactgccagcccatcaccactgtgacggcttctgcctccgtgactgtcgccgtgcacccgccgcctgtccctgggcctgggcggaacccccgagggggactctgcccaggctaccctgagactgaccacggcctgtttgaggacccccacgtgcctttccacgtccggtgtgagaggagggattcgaaggtggaagtcattgagctgcaggacgtggaatgcgaggagaggccccggggaagcagctccaactgagggtgattaaaatctgaagcaaagaggccaaagattggaaaccccccacccccacctctttccagaactgcttgaagagaactggttggagttatggaaaagatgccctgtgccaggacagcagttcattgttactgtaaccgattgtattattttgttaaatatttctataaatatttaagagatgtacacatgtgtaatataggaaggaaggatgtaaagtggtatgatctggggcttctccactcctgccccagagtgtggaggccacagtggggcctctccgtatttgtgcattgggctccgtgccacaaccaagcttcattagtcttaaatttcagcatatgttgctgctgcttaaatattgtataatttacttgtataattctatgcaaatattgcttatgtaataggattattttgtaaaggtttctgtttaaaatattttaaatttgcatatcacaaccctgtggtagtatgaaatgttactgttaactttcaaacacgctatgcgtgataatttttttgtttaatgagcagatatgaagaaagcacgttaatcctggtggcttctctaggtgtcgttgtgtgcggtcctcttgtttggctgtgcgtgtgaacacgtgtgtgagttcaccatgtactgtactgtgattttttttttgtcttgttttgtttctctacactgtctgtaacctgtagtaggctctgacctagtcaggctggaagcgtcaggatatcttttcttcgtgctggtgagggctggccctaaacatccacctaatcctttcaaatcagcccggcaaaagctagactctcctcgtgtctacggcatctcttatgatcattggctgccatccaggaccccaatttgtgcttcagggggataatctccttctctcggatcattgtgatggatgctggaacctcagggtatggagctcacatcagttcatcatggtgggtgttagagaattcggtgacatgcctagtgctgagccttggctgggccatgagagtctgtatactctaaaaagcatgcagcatggtgcccctcttctgaccaacacacacacgacccctcccccaacacccccaaattcaagagtggatgtggccctgtcacaggtagaaaaacctatttagttaattctttcttggcccacagtctcccagaaatgatgttttgagtccctatagtttaaactccctctcttaaatggagcagctggttgaggctttctagatctgtttgcatcttctttaaaactaagtggtgagcatgcattgtggtgtagaggcaggcattatgtaggataagagctccggggggattcttcatgcaccagtgtttagggtacgtgcttcctaagtaaatccaaacattgtctccatcctccccgtcattagtgctctttcaatgtgatgtgggaaagcaggaggatggacacaccccactgaaagatgtaggcaggggcaggtctctcaaccaggcatatttttaaaagttgcttctgtactggttctcgaagcacccagtgtcggctccccatccaaatccacaccagcaccttgttacagacaagaagtcagaggaaagggcggggtccctgcagggctgaagcctaagctactgtgaggcgctcacgagtggcagctcctgttactcccttttaaattacctgggaaatcttaacagaaaggtaatgggcccccagaaatacccacagcatagtgacctcagaccctgatactcaccacaaaacttttaagatgctgattgggagccgcttgtggctgctgggtgtgtgtgtgtgtgtgtgcgtgcgtgcgtgtgtgtgtgtctctgctggggaccctggccacccccctgctgctgtcttggtgcctgtcacccacatggtctgccatcctaacacccagctctgctcagaaaacgtcctgcgtggaggagggatgatgcagaattctgaagtcgacttccctctggctcctggcgtgccctcgctcccttcctgagcccagctcgtgttgcgccggaggctgcgcggcccctgatttctgcatggtgtagaactttctccaatagtcacattggcaaagggagaactggggtgggcggggggtggggctggcagggaattagaatttctctctctcttttaatagttttattttgtctgtcctgtttgttcatttggatgttttaatttttaaaaaaaaaaaaactttgctgatatttataattttgtatcataagaatgttttcctctacagtatttgtcatgccagtttataacaaaaaaaaatgcagggattttatttctattggaaacattacagctatgttttacttttggacagaatttttatttgtatagagtgcttactaatgttaaatagttcagagtatataacatttacattaaggactcatggtaggttttagggtaaggagtttaaaggaaataaatattcaaactgggtctcattgccaattttggtggaaatgagtttgtgtcatttcaattacaaagataaaagtatgccatataatttatttatatgaagatttatttttgtagtgtacatagtagtcatcaagtcttttgacagaagtatatttttaaagaatttatatgtgatgaatccataatgtctggaactttgctgagacatgagtgggcacagttttcattgtaaattacagcaaggaaagaaaatgtttaacagtgttaagagagtcagagcagagtggatattcatgcgattatgaagtgtttattagttaccattggcgacctagcatgcttctcatttcaaaccttggaaggtgaaaatgtacaaactctctaaataattaatgttcaaacactgatagaaattctaacatgaataaaaaataatataacttgttggttatatctttgtttgtagaatgctttttttctttaaaatacttgggagagacagttagtgttggagccatcagaaactgttgtttcatcttgctgaccttgtgacactcttcttttttgctctatctgatggagagagttttaggttttcctcctttttgttttgtataaatagtgggtataagcatagctctgttacatggatgtattacaaagtgggggctgagcttttagtgtgatcatcacccaaatagtgtacattgtacccattaattaatttcttatcacctgaccctatttttgttttctcataccaagaaacttcctaagttaaccatcaaaattagtccttctgtcatctctcttagcatacttatttcccttatgtgatgcctaataatgagggaacataaataaagccaaattcaaagataaaaaaaaaaaaaa

SEQ ID NO: 8 - protein, 1447 aa, Homo sapiens
MASAGNAAEPQDRGGGGSGCIGAPGRPAGGGRRRRTGGLRRAAAPDRDYLHRPSYCDAAFALEQISKGKATGRKAPLWLRAKFQRLLFKLGCYIQKNCGKFLVVGLLIFGAFAVGLKAANLETNVEELWVEVGGRVSRELNYTRQKIGEEAMFNPQLMIQTPKEEGANVLTTEALLQHLDSALQASRVHVYMYNRQWKLEHLCYKSGELITETGYMDQIIEYLYPCLIITPLDCFWEGAKLQSGTAYLLGKPPLRWTNFDPLEFLEELKKINYQVDSWEEMLNKAEVGHGYMDRPCLNPADPDCPATAPNKNSTKPLDMALVLNGGCHGLSRKYMHWQEELIVGGTVKNSTGKLVSAHALQTMFQLMTPKQMYEHFKGYEYVSHINWNEDKAAAILEAWQRTYVEVVHQSVAQNSTQKVLSFTTTTLDDILKSFSDVSVIRVASGYLLMLAYACLTMLRWDCSKSQGAVGLAGVLLVALSVAAGLGLCSLIGISFNAATTQVLPFLALGVGVDDVFLLAHAFSETGQNKRIPFEDRTGECLKRTGASVALTSISNVTAFFMAALIPIPALRAFSLQAAVVVVFNFAMVLLIFPAILSMDLYRREDRRLDIFCCFTSPCVSRVIQVEPQAYTDTHDNTRYSPPPPYSSHSFAHETQITMQSTVQLRTEYDPHTHVYYTTAEPRSEISVQPVTVTQDTLSCQSPESTSSTRDLLSQFSDSSLHCLEPPCTKWTLSSFAEKHYAPFLLKPKAKVVVIFLFLGLLGVSLYGTTRVRDGLDLTDIVPRETREYDFIAAQFKYFSFYNMYIVTQKADYPNIQHLLYDLHRSFSNVKYVMLEENKQLPKMWLHYFRDWLQGLQDAFDSDWETGKIMPNNYKNGSDDGVLAYKLLVQTGSRDKPIDISQLTKQRLVDADGIINPSAFYIYLTAWVSNDPVAYAASQANIRPHRPEWVHDKADYMPETRLRIPAAEPIEYAQFPFYLNGLRDTSDFVEAIEKVRTICSNYTSLGLSSYPNGYPFLFWEQYIGLRHWLLLFISVVLACTFLVCAVFLLNPWTAGIIVMVLALMTVELFGMMGLIGIKLSAVPVVILIASVGIGVEFTVHVALAFLTAIGDKNRRAVLALEHMFAPVLDGAVSTLLGVLMLAGSEFDFIVRYFFAVLAILTILGVLNGLVLLPVLLSFFGPYPEVSPANGLNRLPTPSPEPPPSVVRFAMPPGHTHSGSDSSDSEYSSQTTVSGLSEELRHYEAQQGAGGPAHQVIVEATENPVFAHSTVVHPESRHHPPSNPRQQPHLDSGSLPPGRQGQQPRRDPPREGLWPPPYRPRRDAFEISTEGHSGPSNRARWGPRGARSHNPRNPASTAMGSSVPGYCQPITTVTASASVTVAVHPPPVPGPGRNPRGGLCPGYPETDHGLFEDPHVPFHVRCERRDSKVEVIELQDVECEERPRGSSSN

SEQ ID NO: 9 - DNA, 1725 bp, Homo sapiens
  CDS (1)..(1725)
agtgttgggccagaggacgaaggcgtgtaccagtgcatggctgagaatgcggttggcagtgcccacgctgtggtccaactgaggaccgcccggccagacacaaccctgagacccgggagggataccaagccgattgctgccacaccccccatgccaccctccagacccagcagacctgaccagatgcttcgggaacaaccggggcttgttaagcccccaacgtcgtcggtacagcctacttccctgaagtgcccgggagaagagcaggtagcccctgcagaggcacctatcatcctcagctcaccccggacctccaagacggactcctatgagctggtgtggcggcctcgccatgaggggagcagccggacacccatcctgtactacgtagtgaagcatcgtaaggtcacgaactcctctgacgactggaccatttctggcattccagccaaccagcaccgcctcaccctgaccaggcttgaccctggaagcttgtacgaagtggagatggcagcctacaactgtgctggcgagggccagacagctatggtcaccttccgaacaggacggcggcccaaacctgagatcgtggccagtaaggagcagcagatccagagagatgaccctggtgccagtctccagagcagcagccagcctgaccatggccgcctctcccccccagaagctccagacagacccaccatctccacagcttctgagacctccgtgtacgtaacctggattccccgagggaacggcggcttcccgattcagtctttccgtgtagagtacaagaagctaaaaaaagtgggagattggatactggctaccagtgccatacctccctcgaggctctctgtggagatcacaggcctagagaaaggtatttcttacaagttccgagttcgtgctttgaacatgttaggggagagtgagcccagtgctccctcccggccctacgtggtgtcaggctacagtggcggccgcaagccgaattctgcagatatccatcacactggcggccgctcgagcatgcatctagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaagtga

SEQ ID NO: 10 - protein, 574 aa, Homo sapiens
SVGPEDEGVYQCMAENAVGSAHAVVQLRTARPDTTLRPGRDTKPIAATPPMPPSRPSRPDQMLREQPGLVKPPTSSVQPTSLKCPGEEQVAPAEAPIILSSPRTSKTDSYELVWRPRHEGSSRTPILYYVVKHRKVTNSSDDWTISGIPANQHRLTLTRLDPGSLYEVEMAAYNCAGEGQTAMVTFRTGRRPKPEIVASKEQQIQRDDPGASLQSSSQPDHGRLSPPEAPDRPTISTASETSVYVTWIPRGNGGFPIQSFRVEYKKLKKVGDWILATSAIPPSRLSVEITGLEKGISYKFRVRALNMLGESEPSAPSRPYVVSGYSGGRKPNSADIHHTGGRSSMHLDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 1962 bp, Homo sapiens
  CDS (1)..(1962)
atgacgacgtgccgaagagagcggcctatacttacactgctttggattctcatggccacagcaggctgccttgctgatttgaatgaggttcctcaggtcacagtccagcccatgtccactgtccagaagctgggaggaactgtgatcctgggctgtgtggtggagccaccatggatgaacgtgacttggcgcttcaacggaaaggagctaaatggctctgatgatgctctgggtgtcttcatcacccgtgggacccttgtcattgctgccctcaacaaccacactgtgggacggtaccagtgtgtggcacggatgcctgcaggagctgtggccagtgtgccagccacagtgacgctagccaatctccaggactttaaattagatgtgcagcatgtgattgaagtagacgaggggaacacagccgtcattgcctgccacctgcctgagagccacccaaaagcccaggtccggtacagtgtcaaacaggagtggctggaggcctctagagacaactacctgatcatgccatccgggaatctccaaattgtcaatgccagccaagaggacgaaggcatgtacaagtgtgccgcctacaacccggtgacccaggaagtgaaaacctccggctccggcgacaggctgcgcgtgcgccggtccactgctgaggctgcccgcatcatctacccactggaagcccagaccgtcattgtcaccaaaggccagagtctcatactggagtgtgtggccagtggaatcccaccacctcgagtcacatgggccaaggatgggtccagcattgctgcctataacaagactcgcttcctgctgagtaatttgcttattgacaccaccagcgaggaggactcaggcacctaccgatgtatggccagcaatggggttggggatcctggggcagcagtcatcctctacaatgtccaggtgttcgaaccccctgaggtcacggtggagctgtcccagctggtcatcccatggggccagagtgcaaagctcacctgtgaggttcgaggaaaccccccaccctctgtactatggctgaggaatgcagtgcccctcacctccagccagcgcctccggctgtcacgtagagccctgcgggtggtcagtgttgggccagaggacgaaggcgtgtaccagtgcatggctgagaatgcggttggcagtgcccacgctgtggtccaactgaggaccgcccggccagacacaaccctgagacccggatccatcacactggcggccgctcgatcgagcatgcatctagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaagtga

SEQ ID NO: 12 - protein, 653 aa, Homo sapiens
MTTCRRERPILTLLWILMATAGCLADLNEVPQVTVQPMSTVQKLGGTVILGCVVEPPWMNVTWRFNGKELNGSDDALGVFITRGTLVIAALNNHTVGRYQCVARMPAGAVASVPATVTLANLQDFKLDVQHVIEVDEGNTAVIACHLPESHPKAQVRYSVKQEWLEASRDNYLIMPSGNLQIVNASQEDEGMYKCAAYNPVTQEVKTSGSGDRLRVRRSTAEAARIIYPLEAQTVIVTKGQSLILECVASGIPPPRVTWAKDGSSIAAYNKTRFLLSNLLIDTTSEEDSGTYRCMASNGVGDPGAAVILYNVQVFEPPEVTVELSQLVIPWGQSAKLTCEVRGNPPPSVLWLRNAVPLTSSQRLRLSRRALRVVSVGPEDEGVYQCMAENAVGSAHAVVQLRTARPDTTLRPGSITLAAARSSMHLDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK